(19) United States
(12) Patent Application Publication
(10) Pub. No.: US 2021/0361718 A1
(43) Pub. Date: Nov. 25, 2021

(54) METHODS AND COMPOSITIONS RELATED TO PLATELET RELEASATE AND PLATELET-RICH FIBRIN

(71) Applicant: Biobridge Global, San Antonio, TX (US)

(72) Inventor: Anand Srinivasan, San Antonio, TX (US)

(21) Appl. No.: 17/395,691

(22) Filed: Aug. 6, 2021

Related U.S. Application Data
(63) Continuation of application No. PCT/US2020/017325, filed on Feb. 7, 2020.
(60) Provisional application No. 62/802,623, filed on Feb. 7, 2019.

(51) Int. Cl. A61K 35/28; C12N 5/0775; C12N 5/078

(52) U.S. Cl. CPC A61K 35/28; C12N 5/0663; C12N 5/0644; C12N 2500/14; C12N 2501/115; C12N 2501/11; C12N 2527/00; C12N 2501/15; C12N 2501/165; C12N 2501/135; C12N 2501/12; C12N 2501/21

(57) ABSTRACT

The present disclosure provides compositions and methods comprising human platelet releasate (hPR), a xeno-free media supplement. The disclosure also relates to a cGMP process for the rapid, efficient and large-scale manufacturing of hPR that may be performed in less than 4 hours. The platelet releasate of the current disclosure prevents gelation of growth media alleviating the need for heparin or other anticoagulants. Mesenchymal stem cells expanded in the presence of platelet releasate have demonstrated superior expansion rates and potency compared to commercial supplements including platelet lysates. The releasate has therapeutic and medical applications. The disclosure also relates to compositions and methods related to platelet-rich fibrin.

### CROSS-REFERENCE

This application is a continuation of International Application No. PCT/US2020/017325 filed Feb. 7, 2020, which claims priority to U.S. Provisional Patent Application No. 62/802,623, filed Feb. 7, 2019, each of which are hereby incorporated by reference in their entirety.

### BACKGROUND

### Field of the Invention

The disclosure is generally directed to biotechnology, medical products, and commercial manufacturing of cell culture media supplements. Embodiments pertain to compositions comprising cell culture media supplements and to methods of preparing such supplements from animal-blood derived platelet-rich plasma.

### Background

Mesenchymal Stem cells (MSCs) have found widespread applications in the treatment of trauma, wound care (bone and cartilage regeneration), myocardial infarction, and auto-immune diseases. Additionally, there is a military significance to the use of MSCs in treating casualties, for example.

In order to support clinically relevant dosage in such varying gamut of clinical conditions, it is important to expand cells on a large-scale and under good manufacturing practice (GMP) guidelines without compromising the idiosyncrasy of MSCs. Conventional practices of stem cell expansion are still facing adjourned problems such as long expansion times and use of animal-derived serum. The arduous process of large-scale cell expansion can be ameliorated by the use of bioreactors and automation.

Prevailing conventional practice depends on the use of fetal bovine serum (FBS) as a supplement to the growth medium. FBS plays a pivotal role in actively promoting cell growth and proliferation. Although the risk of exposure to zoonoses is small, the regulatory efforts to scrutinize for xenogeneic pathogens remain high. Additionally, the risk of immune reaction to animal-derived proteins mandates stringent regulations. In order to alleviate these issues, FDA and other regulatory authorities have encouraged the use of xeno-free alternatives to FBS. However, the use of serum-free, chemically defined media is not cost-effective for large-scale expansion.

Several reports on clinical-grade cell expansion have indicated the use of human platelet lysate (hPL) as a xeno-free supplement to the growth medium. Although hPL can sustain the growth of MSC without affecting their immunophenotype, recent reports have indicated a rather inconsistent effect imparted on the population-doubling and immunosuppressive properties of MSCs. Such incongruity could be attributed to differences in the methods of hPL production. In addition to this, supplementing hPL triggers gelation of growth medium, thereby creating less-conducive environment and expansion rates for conventional cell culture. Often this issue is mitigated by the addition of heparin, an anticoagulant-derived from porcine; ultimately defeating previous efforts of maintaining xeno-free expansion conditions.

Currently, there is a need for more efficient protocols, methods, and systems for the commercial scaling up of hPL production, wherein the need for heparin or other anticoagulants is alleviated. Moreover, there is a need for a protocol that can be scaled up under good manufacturing practice guidelines to provide commercial quantities of hPL. There is a need for such a protocol to be cost-effective, to be conducted in a closed system to minimize the risks of contamination, to yield a desirable composition that is rich in growth factors and cytokines and for the entire process to take the least amount of time from start to finish.

### SUMMARY OF THE DISCLOSURE

The current embodiments include, inter alia, compositions and methods relating to platelet releasate (hPR), particularly the manufacture of hPR on a large scale. A releasate, in accordance with the current disclosure, is a product or extract obtained from cells that are manipulated (e.g., degranulated) according to methods of the current disclosure; the releasate can be used as a supplement in cell culture media to culture or expand cells. hPR prevents gelation of growth medium, thereby alleviating the need for heparin or other anticoagulants. Additionally, there are embodiments including compositions and methods concerning platelet-rich fibrin, particularly the manufacture of platelet-rich fibrin on a large scale.

In some embodiments, hPR is enriched with growth factors and comprises less than about 0.05 mg/dL fibrinogen. In some embodiments, hPR comprises FGF-basic at a level of at least about 300 pg/ml. In some embodiments, hPR comprises SDF-1α at a level of about 50-20 pg/ml. In some embodiments, the hPR manufacturing process is carried out on an industrial-scale and/or in a closed system that can yield up to 10, 50, 100 liters or even as high as hundreds of liters of hPR. Stem cells such as mesenchymal stem cells expanded in the presence of hPR have demonstrated superior expansion rates compared to their commercial counterparts.

Here, embodiments include compositions of platelet releasate, media supplements comprising platelet releasate, xeno-free serum supplements comprising platelet releasate, cell cultures expanded on platelet releasate, cell culture media comprising platelet releasate, sterilized platelet releasate, methods of sterilizing releasate, lyophilized releasate, therapeutic compositions, pharmaceutical formulations, methods of preparing platelet releasate, methods of manufacturing platelet releasate, methods of industrially producing platelet releasate, methods of commercially scaling-up of bone marrow derived mesenchymal stem cells, methods of expanding cells, methods of expanding cells on an industrial scale, methods of expanding cells for regenerative medicine, closed systems for producing platelet releasate, open systems for producing platelet releasate, methods of reducing the process time for the production of platelet releasate, and methods of treating a subject using mesenchymal stem cells expanded on hPR.

Any one or more of the methods of the current disclosure may comprise or exclude or consist of or consist essentially of one or more of the following steps: obtaining a source or solution of whole-blood, platelets or platelet-rich-plasma (PRP) or platelet concentrates, weighing, pooling, mixing, agitating, shaking, activating, aggregating, centrifuging, freezing, thawing, balancing, filtering, double filtering, incubating, triggering, concentrating, yielding, clotting, gelling, separating, and packaging,

In some aspects, the disclosure relates to a method for preparing a platelet releasate, the method comprising the steps of: i) obtaining platelets from human blood thereby obtaining a platelet-rich-plasma (PRP), ii) adding CaCl₂) to the PRP to a final concentration of greater than 25 mM, and iii) agitating the CaCl₂/PRP mixture for less than 6 hours thereby forming a clot and a releasate. In some embodiments of the method the releasate comprises fibrinogen at a level of less than about 0.05 mg/dL. In some embodiments, the method further comprises, prior to ii), concentrating by removing excess plasma.

In some embodiments of the methods of the current disclosure, the releasate comprises FGF-basic at a level of at least about 300 pg/ml. In some embodiments, releasate comprises FGF-basic at a level of about 300-550 pg/ml. In other embodiments, the releasate comprises FGF-basic at a level of about 350-520 pg/ml. Yet in further embodiments, the releasate comprises FGF-basic at a level between about 400-500 pg/ml. In some embodiments, the releasate comprises FGF-basic at a level about 450 pg/ml. In some embodiment the releasate comprises FGF-basic at a level of about, at least about, or at most about 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 509, 510, 515, 520, 525, 530, 535, 540, 545, or 550 pg/ml or any range derivable therein.

In some embodiments of the methods of the current disclosure, the releasate comprises about 5.0-20 pg/ml of SDF-1α. In some embodiments, the releasate comprises about 7.0-15 pg/ml of SDF-1α. In some embodiments, the releasate comprises about 8.0-14 pg/ml of SDF-1α. In other embodiments, the releasate comprises about 9.0-12.0 pg/ml of SDF-1α. In further embodiments, the releasate comprises SDF-1a at a concentration of about, at least about, or at most about 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0 pg/ml or any range derivable therein.

In some embodiments of the methods of the current disclosure, the final concentration of CaCl₂) is greater than about 30 mM. In some embodiments, the final concentration of CaCl₂) is between about 25-80 mM. In some embodiments, the final concentration of CaCl₂) is between about 30-50 mM. In further embodiments, the final concentration of CaCl₂) is between about 40-47 mM. Yet in further embodiments the final concentration of CaCl₂) is about 45 mM. In some embodiments of the methods of the current disclosure, the final concentration of CaCl₂) is 80 mM. In some embodiments, the CaCl₂) final concentration is about 80-90 mM. In some embodiments, the final concentration of CaCl₂) is at least about, at most about, or about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 100 mM or more (or any value derivable within). In some embodiments, as can be realized by a person of skill in the art, the concentration will vary depending on the choice of calcium salt. In some embodiments, the calcium salt is or comprises calcium gluconate, calcium citrate, calcium phosphate, calcium chloride, calcium acetate, calcium carbonate, or other calcium salts.

In some embodiments, methods for preparing a platelet releasate from beginning to end takes no more than 4 hours or between 2.5-4 hours. In some embodiments, the duration of the method is at least, at most or about 2, 2.5, 3, 3.5, 4.0, 4.5, 5.0, 5.5, 6.0, 6.5, 7.0, 7.5, or 8.0 hours or any range derived therein. In some embodiments, the duration of the method is or is about 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 minutes and any range derivable therein. The initiation of the method, in some embodiments, begins with, testing a blood type of a blood sample, obtaining blood from a subject or biological sample, obtaining platelets from a biological sample, isolating platelets from a biological sample, thawing a sample of frozen platelets, or isolating plasma from a biological sample and, in some embodiments, concludes with agitating a mixture containing platelets to generate a releasate, centrifuging a releasate, filtering a releasate, storing a releasate, freezing a releasate, mixing a releasate with a cell culture medium, or culturing cells with a releasate. In some embodiments, steps i), ii), and iii) are understood to be, respectively, obtaining (e.g., isolating) platelets of the human blood; adding CaCl₂) to the PRP, and agitating the CaCl₂/PRP mixture to form a clot. In some embodiments, step i) further comprises concentrating the platelets by removing excess plasma. In some embodiments, the duration of steps i), ii)) and iii) is 2-6 hours (e.g., 3-4 hours). In some embodiments, the duration of steps i), ii)), iii) and iv) is 2-6 hours (e.g., 3-4 hours).

In some embodiments of the methods of the current disclosure, the CaCl₂/PRP mixture is agitated for less than about 4 hours. In some embodiments, the CaCl₂/PRP mixture is agitated for less than about 6 hours. In other embodiments, the CaCl₂/PRP mixture is agitated for less than about 180 minutes. In further embodiments, the CaCl₂/PRP mixture is agitated for about 30-150 minutes or from 45-135 minutes. Yet in other embodiments, the CaCl₂)/PRP mixture is agitated from 60-90 minutes. In some embodiments the CaCl₂/PRP mixture is agitated at least for or at most for or for 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240 minutes or any range derivable therein.

In some embodiments of the methods of the current disclosure, the CaCl₂/PRP mixture is agitated at a certain rotation per minute that is between 50-500 rpm. In some embodiments of the methods of the current disclosure, the CaCl₂/PRP mixture is agitated at a certain rotation per minute that is between 150-350 rpm. In some embodiments, the CaCl₂/PRP mixture is agitated at an rpm that is at least or at most or about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500 rpm, or any value derivable therein. In some embodiments of the methods of the current disclosure that comprise batch sizes of about 50 or 100 L, the rpm may be at least about, at most about, or about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 215, 220, 225, 230, 235, 240, 245, 250 rpm, or any value derivable therein.

The current disclosure relates to human platelet releasate that is derived from plasma (e.g., platelet-rich plasma). In some embodiments, the platelets are at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30-days post donation or any value derivable therein. A “day” refers to 24 hours or less such that 3 days means 72 hours or less in time. In some embodiments the releasate is derived from fresh-platelets—(i.e., within 24 hours of donation). In some embodiments, the platelets are expired. In some embodiments, the expired platelets are at least or at most 7-days post donation, 14-days post donation, 21-days post donation, 30-days post donation. In some embodiments, the platelets are stored at room temperature (23-25° C. for example). In some embodiments, the platelets are cold-stored platelets (4-20° C. for example). In some embodiments, the platelets are frozen. In some embodiments, the platelets are frozen at about −55 to −80° C. or any temperature with this range. In some embodiments, the plasma is further concentrated to yield platelet concentrates. In some embodiments, a platelet concentrate has a concentration level that is higher by about, at least about or at most about 20, 25, 30, 35, 40, 45, 50, 55, 60, 65, 70, 75 or more percent (or any range derivable therein) or 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20× or more (or any range derivable therein). In some embodiments, the plasma is not concentrated.

In some embodiments, the releasate is derived from mammalian platelet rich plasma. In some embodiments, the platelets are derived from human platelet rich plasma. In some embodiments, the platelets are derived from equine, canine, bovine, chicken, feline, porcine, rabbit, dolphin, ovine, murine, rat, or simian blood. In some embodiments, the platelets are from a sport animal, a farm animal, or a pet.

In some embodiments, the methods of the current disclosure further comprise a step of separating the clot from the releasate such that less than about 10, 9, 8, 7, 6, 5, 4, 3, 2, 1% or less of the clot detectably remains in the separated releasate. In some embodiments, the clot is a fibrin clot. In some embodiments, the clot encapsulates cells from blood and cell debris.

In some embodiments, the methods of the current disclosure further comprise the step of filtering the releasate. In some embodiments, the releasate is filtered using a filter that is a 0.45-1.0 micron filter. In some embodiments, the filter is 0.45, 0.50, 0.55, 0.60, 0.65, 0.70, 0.75, 0.80, 0.85, 0.90, 0.95, 1.0 microns or any size derivable therein. In some embodiments, the filter is a 3-10 micron filter. In some embodiments the filter is at least or at most 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0 microns or any size derivable therein. In some embodiments, the filter is a 170-260 micron filter. In some embodiments the filter is a 170-260 micron filter. In some embodiment, the filter is at least or at most 170, 175, 180, 185, 190, 195, 200, 205, 210, 215, 220, 225, 230, 235, 240, 245, 250, 255, 260 micron filter or any range derivable therein.

In some embodiments of the methods for preparing a platelet releasate, steps i), ii), and iii) are performed in a closed bag. In some embodiments, steps i), ii), and iii) are performed in a closed system. In some embodiments, steps i), ii), and iii) are performed on an industrial scale in a closed system. In some embodiments of any of the method presented herein, the methods further comprise step iv) filtering the releasate. In some embodiments steps i), ii), iii) and iv) are performed in a closed bag. In some embodiments the filtration step is also part of a closed system. In some embodiments some or all of steps i), ii), iii) and iv) are performed in a closed system. In some embodiments, some or all of steps i), ii), iii), and iv) are performed in an open system.

In some embodiments of the methods for preparing a platelet releasate, the releasate is produced at a yield of about 0.2-10 liters. In some embodiments, the releasate is produced at a yield of about 4.5-10 liters. In some embodiments the yield is at least, at most or about 2.0, 2.5, 3.0, 3.5, 4.5, 5.0, 5.5, 6.0, 7.0, 7.5, 8.0, 8.5, 9.0, 9.5, or 10 liters or any value derivable therein. In some embodiments of the methods for preparing a platelet releasate, the releasate is produced at a yield of about 0.2-100 liters. In some embodiments, the releasate is produced at a yield of about 50-100 liters. In some embodiments the yield is at least, at most or about 2.0, 2.5, 3.0, 3.5, 4.5, 5.0, 5.5, 6.0, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 liters, or any value derivable therein.

In some aspects, the current disclosure relates to a releasate composition produced by any of the methods described herein. The amount of the releasate composition can be about, at most about or at least about 2.0, 2.5, 3.0, 3.5, 4.5, 5.0, 5.5, 6.0, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 liters, or any value derivable therein.

In other aspects, the current disclosure relates to a cell culture medium comprising a releasate produced by any of the methods described herein. In some embodiments, the cell culture medium is free from added heparin or other anticoagulants.

Further aspects of the current disclosure relate to a method of culturing cells (e.g., stem cells), the method comprising the step of expanding the cells on a cell culture medium comprising a releasate from a human or mammalian platelet-rich plasma. In some embodiments, the releasate comprises fibrinogen at a level of less than about 0.05 mg/dL. In some embodiments, the releasate comprises FGF-basic at a level of less than about 300 pg/ml. In some embodiments the releasate comprises SDF-1a at a level of about 5.0-20 pg/ml. In some embodiments, the cell culture medium is free from added heparin. In some embodiments, the cells are pluripotent stem cells (PSC), induced pluripotent stem cells (iPSC), hematopoietic stem cells (HSC), bone-marrow-derived-mesenchymal stromal/stem cells (BM-MSC), Adipose-derived Mesenchymal Stromal/stem Cells (ADP-MSC), T-Cells, B-cells, Natural Killer cells, Dendritic cells, Peripheral Blood-derived Mononuclear Cells, cancer cells cancer stem cells, Chinese Hamster Ovarian (CHO), umbilical cord-blood-derived cells, umbilical cord-blood tissue-derived cells, placenta-derived cells, retinal cells, neurons, fibroblasts, epithelial cells, endothelial cells or keratinocytes or fibroblasts, osteoblasts, adipocytes, chondrocytes, endothelial cells, cells of the immune system, T-cells, B-cells, NK cells, engineered cells (e.g., chimeric antigen receptor (CAR) T-cells), or neurons. In some embodiments, releasate stimulates release of components from cells. In some embodiments, releasate stimulates release of exosomes, extracellular vesicles, proteins, nucleic acids, or a combination thereof. In some embodiments, releasate stimulates release of exosomes. In some embodiments, the method comprises collecting components released from cells expanded on a cell culture medium comprising a releasate.

Further aspects of the current disclosure relate to a method of treating a mammalian subject comprising administering to the subject a composition comprising a population of stem cells, wherein the stem cells have been cultured with the releasate compositions described herein. In some embodiments, the stem cells are mesenchymal stem cells. In some embodiments, the cells are pluripotent stem cells (PSC) or induced Pluripotent stem cells (iPSC) or any associated lineages of differentiation. In some embodiments, the stem cells are bone marrow-derived mesenchymal stem cells, bone-marrow-derived-mesenchymal stromal/stem cells (BM-MSC), adipose-derived mesenchymal stromal/stem Cells (ADP-MSC), T-Cells, B-cells, Natural Killer cells, Dendritic cells, Peripheral Blood-derived Mononuclear Cells. In some embodiments, the stem cells are autologous. In other embodiments, the stem cells are allogenic. In some embodiments, the stem cells are xenogenic. In some embodiments, the stem cells are engineered or manipulated cells.

In some embodiments, the population of stem cells comprise 2-100% of cells of a particular lineage of differentiation. In some embodiments, the methods of culturing stem cells with releasate compositions of the current disclosure result in a population of cells in which the percentage of a certain lineage is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the total population or any value derivable therein.

Further aspects of the current disclosure relate to a method of treating a subject comprising administering to the subject a composition administering to the subject a composition comprising stem cells, wherein the stem cells have been cultured with the releasate from human blood-derived platelets wherein the cells have been cultured with the releasate compositions described herein.

Additional aspects of the current disclosure relate to a method of treating a subject comprising administering to the subject a composition comprising stem cells, wherein the stem cells have been cultured with a releasate from human blood-derived platelets, wherein the releasate comprises fibrinogen at a level of less than about 0.05 mg/dL. In some embodiments, the subject suffers from bone disease, bone defect, bone injury, osteoporosis, osteoarthrosis or spinal cord injury. In some embodiments the subject suffers from cartilage disease or cartilage defect or cartilage injury. In some embodiments, the subject suffers from a bone, tendon, cartilage, or muscle injury. In some embodiments, the subject suffers from periodontal disease. In some embodiments, the subject suffers from an autoimmune disease. In some embodiments, the subject suffers from myocardial infarction. In some embodiments, the subject suffers from or has suffered from graft versus host disease (GvHD), Chronic Obstructive Pulmonary Disease (COPD), acute respiratory distress syndrome (ARDS), poly-trauma, a systemic infection, or cancer. In some embodiments, the subject is a non-human animal subject.

Further aspects of the current disclosure relate to a composition comprising a releasate from human or mammalian blood-derived platelets. In some embodiments, the releasate comprises fibrinogen at a level of less than about 0.05 mg/dL. In some embodiments, the releasate comprises FGF-basic at a level of less than about 300 pg/ml. In some embodiments the releasate comprises SDF-1a at a level of about 5.0-20 pg/ml. In some embodiments, the releasate comprises microvesicles from platelets. In some embodiments, the releasate comprises exosomes from platelets.

Some aspects of the current disclosure relate to formulations comprising the releasate described herein. The formulations may be used for therapeutic purposes such as for the treatment of diseased or damaged tissue including but not limited to bone, muscle, skin, nerves, tendons, connective, ocular, periodontal or cardiovascular tissues. In some embodiments, formulations are used for therapeutic purposes for treating or alleviating ocular conditions, including, for example, dry eye. In some embodiments, a formulation of the present disclosure may be an artificial or synthetic tear formulation.

In some embodiments of the compositions, formulations and methods of the current disclosure, the releasate comprises FGF-basic at a level of at least about 300 pg/ml. In some embodiments, the releasate comprises FGF-basic at a of about 300-550 pg/ml, 350-520 pg/ml or about 400-500 pg/ml. In some embodiments, the releasate comprises FGF-basic at about 450 pg/ml. In some embodiments, the FGF-basic is, is at least, or is at most or is about 300, 310, 320, 330, 340, 350, 360, 370, 380, 390, 400, 410, 420, 430, 440, 450, 460, 470, 480, 490, 500, 510, 520, 530, 540, 550 pg/ml.

In some embodiments of the methods or compositions of the current disclosure, the releasate comprises globulins, albumin, growth factors, cytokines, interleukins, interferons, chemokines, hormones, and glycoproteins such as fibronectin, vitronectin, laminin or other compounds. In some embodiments, the releasate comprises microvesicles or extracellular vesicles. In some embodiments, the releasate comprises exosomes. In some embodiments, the releasate comprises TGF beta 1, EGF, bFGF, PDGF-AA, PDGF-BB, PDGF-AB, SDF-1α, VEGF, or HGF. In some embodiments, the releasate comprises one or more of the proteins listed in Table 1. In some embodiments, the releasate does not comprise one or more of the proteins listed in Table 1. In some embodiments, the releasate comprises 1, 2, 3, 4, 5, 6, 7, 8, 9, 10, 11, 12, 13, 14, 15, 16, 17, 18, 19, 20, 30, 40, 50, 100, 150, or 200, or any value derivable therein, of the proteins listed in Table 1. In some embodiments, the releasate does not comprise 1, 2, 3, 4, 5, 6, 7, 8, 9, 10, 11, 12, 13, 14, 15, 16, 17, 18, 19, 20, 30, 40, 50, 100, 150, or 200, or any value derivable therein, of the proteins listed in Table 1. In some embodiments, the releasate comprises less than 0.01, 0.02, 0.03, 0.04, 0.05, 0.06, 0.07, 0.08, 0.09, 0.1, 0.2, 0.3, 0.4, or 0.5 pg/ml of one or more of the proteins listed in Table 1. In some embodiments, the releasate comprises less than 0.1 pg/ml of one or more of the proteins listed in Table 1.

Some aspects of the disclosure relate to methods of treating casualties using mesenchymal stem cells, endothelial cells, fibroblasts, cells of the immune system (for example, T-cells, B-cells, NK cells or other engineered cells), or neurons cells expanded on hPR. The steps and embodiments discussed in this disclosure are contemplated as part of any of these methods.

Further aspects of the disclosure relate to methods of promoting cell adhesion, cell differentiation or cell expansion in tissue culture comprising coating tissue culture containers with a composition comprising a releasate from human blood-derived platelets, the releasate comprising fibrinogen at a level of less than about 0.05 mg/dL. In some embodiments, the container is a petri dish, a flask or a bioreactor.

Further aspects of the disclosure relate to methods of preparing osteobiologic material, comprising a step of adding to said osteobiologic material a composition comprising a releasate from human blood-derived platelets, the releasate comprising fibrinogen at a level of less than 0.05 mg/dL. In some embodiments, the osteobiologic material is an osteobiologic graft material, a bone sponge, or a bone putty. In some embodiments, the osteobiologic material further includes mammalian tissue, engineered cells or manipulated cells.

In some aspects, the disclosure relates to methods of preparing coagulation agents comprising a step of adding to said coagulation agents a composition comprising a releasate from human blood-derived platelets, the releasate comprising fibrinogen at a level of less than about 0.05 mg/dL.

In some aspects, the disclosure relates to methods for preparing platelet-rich fibrin from mammalian blood-derived platelets, the method comprising the steps of: i) obtaining (e.g., isolating) platelets of the human blood thereby obtaining platelet-rich-plasma (PRP); ii) adding CaCl₂) to the PRP to a final concentration of greater than 25 mM; and iii) agitating the CaCl₂/PRP mixture for less than 6 hours thereby forming a fibrin clot and a supernatant; iv) adding an anti-fibrinolytic agent to prevent fibrinolysis; and v) removing the supernatant to obtain the platelet-rich fibrin. In some embodiments, the method further comprises concentrating by removing excess plasma In some embodiments the mammalian blood is human, equine, canine, feline, porcine, bovine, chicken, feline, porcine, rabbit, dolphin, ovine, murine, rat, simian blood, from a sport animal, from a farm animal, or from a pet. Any steps or methods used to produce a releasate may be used, in some embodiments, to produce a platelet-rich fibrin. Such steps and methods may further include adding an anti-fibrinolytic agent to prevent fibrinolysis; and then removing the supernatant to obtain the platelet-rich fibrin. In other aspects, the current disclosure is related to platelet-rich fibrin composition produced by the methods described herein. In some embodiments, the platelet-rich fibrin does not include detectable levels of thrombin.

Aspects are directed to kits comprising a platelet releasate from mammalian blood-derived platelets. Kits may comprise additional components, e.g., cell culture medium or other cell culture additives. In some embodiments, the kit comprises instructions for use of the releasate as a cell culture medium supplement.

It is contemplated that any method or composition or method step described herein can be implemented with respect to any other method or composition described herein and that different embodiments may be combined. In certain embodiments, a method for generating platelet-rich fibrin involves one or more steps used to produce a releasate, and vice versa.

Use of the one or more method steps or compositions may be employed based on any of the methods described herein. Other embodiments are discussed throughout this application. Any embodiment discussed with respect to one aspect of the disclosure applies to other aspects of the disclosure as well and vice versa. The embodiments in the Examples section are understood to be embodiments that are applicable to all aspects of the technology described herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1O show the quantities and identities of various growth factors cytokines, interleukins, interferons in example releasates of the current disclosure compared with plasma, platelet lysates, defined chemical media, or media supplied with FBS. Each value represents a dilution factor of 2.0, i.e. each value shown is half of the true concentration value.

FIG. 1P shows a table correlating the sample types with the sample IDs used in FIGS. 2-6. “ACR” indicates releasate obtained from “Acrodose” pooled platelets. “APH” indicates releasate obtained from apheresis of a single individual. “pre” indicates pre-filtration samples. “post” indicates post-filtration samples. “RT” indicates a sample from platelets at room temperature. “RT” indicates a sample from frozen platelets.

FIG. 2 is a 3-dimensional bar graph depicting the values of various growth factors and cytokines in example releasate of the current disclosure. Each value represents a dilution factor of 2.0, i.e. each value is half of the true concentration value.

FIG. 3 is a 3-dimensional bar graph depicting the values of various growth factors and cytokines in example releasate of the current disclosure. Each value represents a dilution factor of 2.0, i.e. each value shown is half of the true concentration value.

FIG. 4 is a 3-dimensional bar graph depicting the values of various growth factors and cytokines in example releasate of the current disclosure. Each value represents a dilution factor of 2.0, i.e. each value shown is half of the true concentration value.

FIG. 5 is a 3-dimensional bar graph depicting the values of various growth factors and cytokines in example releasate of the current disclosure. Each value represents a dilution factor of 2.0, i.e. each value shown is half of the true concentration value.

FIG. 6 is a 3-dimensional bar graph depicting the values of various growth factors and cytokines in example releasate of the current disclosure. Each value represents a dilution factor of 2.0, i.e. each value shown is half of the true concentration value.

FIG. 7 shows a Principal Component Analysis comparing example releasate of the current disclosure with plasma, platelet lysates, defined chemical media, and media supplied with FBS.

FIG. 8 is a heat map comparing biomarkers from example releasate of the current disclosure with controls. The controls include plasma, commercial platelet lysates, chemically defined media and media supplemented with fetal bovine serum.

FIGS. 9A-9B depict results obtained testing an example method of preparing platelet releasate in accordance with the current disclosure using various concentrations of CaCl₂). The concentration of CaCl₂) in the range of 25-80 mM gives well-defined fibrin clots that encapsulate cell debris.

FIG. 10 shows the amount of fibrinogen in example releasate of the current disclosure. “ACR” indicates releasate obtained from “Acrodose” pooled platelets. “APH” indicates releasate obtained from apheresis of a single individual. “pre” indicates pre-filtration samples. “post” indicates post-filtration samples. “RT” indicates a sample from platelets at room temperature. “RT” indicates a sample from frozen platelets.

FIGS. 11A-11C show the effect of CaCl₂) concentration in hPR on MSC doubling. Increasing CaCl₂) concentrations are shown from left to right.

FIGS. 12A-12C show the effect of the duration of platelet agitation on MSC doubling. Increasing agitation duration is shown from left to right, alternating between Acrodose platelets and apheresis platelets.

FIG. 13 shows the effect of hPR centrifugation duration on MSC doubling. ACR are Acrodose platelets and APH designate platelets obtained by apheresis. Increasing centrifugation duration is shown from left to right, alternating between Acrodose platelets and apheresis platelets.

FIG. 14 shows the values of various growth factors obtained in example releasate of the current disclosure. Each value represents a dilution factor of 2.0, i.e. each value shown is half of the true concentration value. The releasate is from room temperature expired platelets or from frozen expired platelets.

FIG. 15 shows the values of total protein, albumin and globulin obtained in example releasate of the current disclosure. The releasate is from room temperature expired platelets or from frozen expired platelets.

FIGS. 16A-16C show a comparison data between hPRs of the current disclosure and controls. FIG. 16A shows total protein and growth factors values. FIG. 16B shows the effect of hPR on MSC expansion. FIG. 16C depicts the luminescence intensity obtained for hPR and controls.

FIG. 17A. shows a scheme in accordance with one embodiment of the current disclosure for cGMP small-scale manufacturing. FIG. 17B. shows an example of a bag for a 30 ml volume small scale yield.

FIG. 18 shows a scheme in accordance with one embodiment of the current disclosure for cGMP small-scale manufacturing.

FIG. 19 shows comparison data pertaining to total proteins, globulins, and albumins between an example human releasate in accordance with the current disclosure and a commercial lysate.

FIGS. 20A-20B show comparison data pertaining to growth factors between an example human releasate in accordance with the current disclosure and a commercial lysate. Each value represents a dilution factor of 2.0, i.e. each value shown is half of the true concentration value.

FIG. 21 shows comparison data between an example human releasate in accordance with the current disclosure and a commercial lysate. FIG. 21A shows MSC doubling after 5 days for cells grown in DMEM growth media mixed with supplements. FIG. 21B depicts the luminescence intensity obtained for hPR and controls. Controls include commercial platelet lysates, chemically defined media and media supplemented with fetal bovine serum. Results show that it takes at least twice as much of a commercial lysate to produce a similar population doubling level (PDL) as releasate of the current disclosure. The results also show the superiority of disclosed releasate as a media supplement on the expansion of MSC.

FIGS. 22A-22F show a heatmap comparing biomarkers from example releasate of the current disclosure (Releasate F1 and Releasate F2) with controls. The controls include human serum (AB Serum), commercial platelet lysates (Lysate A, Lysate B, Lysate C), and media supplemented with fetal bovine serum (FBS). These data show that the composition of the releasates is different from platelet lysates. FIG. 22A shows a heatmap of all biomarkers tested, grouped into five groups based on similarity. FIG. 22B shows the section of the heatmap for the biomarkers in group 1. FIG. 22C shows the section of the heatmap for the biomarkers in group 2.

FIG. 22D shows the section of the heatmap for the biomarkers in group 3. FIG. 22E shows the section of the heatmap for the biomarkers in group 4. FIG. 22F shows the section of the heatmap for the biomarkers in group 5.

FIG. 23 shows population doubling levels of mesenchymal stem cells (MSCs) from three different donors cultured with various additives: fetal bovine serum (FBS), human AB plasma (AB serum), commercial lysates (Lysate A, Lysate B, Lysate C), or example releasate of the present disclosure (Releasate F1, Releasate F2).

FIG. 24 shows the results of a protein concentration assay performed on cells expanded with various supplements: fetal bovine serum (FBS), human AB plasma (AB serum), commercial lysates (Lysate A, Lysate B, Lysate C), example releasate of the present disclosure (Releasate F1, Releasate F2), or pooled plasma.

FIGS. 25A and 25B show a scanning electron microscope (SEM) image of resting (FIG. 25A) and activated (FIG. 25B) platelets.

FIG. 26A is an SEM image of activated platelets showing topical changes due to the activation process (CaCl₂) addition, agitation). FIG. 26B is a transmission electron microscopy (TEM) image showing a cross-section of activated platelets demonstrating degranulation, with vesicles (e.g., alpha-granules, dense-granules, mitochondria, etc.) exiting the cells either releasing their contents into the surrounding medium or remaining intact.

FIG. 27 is a TEM image of a cross-section of an activated platelet, where granules (e.g., alpha-granules, dense-granules, mitochondria, etc.) are observed moving towards the periphery of the cell and detaching or releasing as independent vesicles (arrow marks, top left and top right). Vesicles outside the cell are shown releasing their contents into the surrounding medium or remaining intact (arrow marks, bottom left).

FIG. 28 is a TEM image of a cross-section of an activated platelet showing degranulation, where granulates are observed moving towards the periphery of the cell and detaching or releasing as independent vesicles. The vesicles are shown to vary in size (arrow marks, bottom left) and content. The cell is also shown to release factors into the surrounding medium (arrow mark, middle left).

FIG. 29 shows a TEM image of a cross-section of platelets after degranulation, and demonstrates that the cells are devoid of granules (e.g., alpha-granules, dense-granules, mitochondria, etc.) yet remain intact (i.e., are not lysed). Also shown are vesicles outside the cells which are trapped along with fibrin (dark strands).

FIG. 30A shows an SEM image of platelets embedded or encapsulated in a fibrin matrix. FIG. 30B shows strands of fibrin polymer with platelets entrapped within the mesh.

FIG. 31 shows results from expansion of bone marrow-derived MSCs (BM-MSCs) and MSC marker analysis.

FIGS. 32A-32G show microscopy images of BM-MSCs expanded in basal growth medium supplemented with one of various supplements, as shown, to evaluate adipogenesis potential of the BM-MSCs.

FIGS. 33A-33G show microscopy images of BM-MSCs expanded in basal growth medium supplemented with one of various supplements, as shown, to evaluate chondrogenesis potential of the BM-MSCs.

FIGS. 34A-34G show microscopy images of BM-MSCs expanded in basal growth medium supplemented with one of various supplements, as shown, to evaluate osteogenesis potential of the BM-MSCs.

FIG. 35 shows results from an immunomodulation experiment in which BM-MSCs were expanded in basal growth medium supplemented with one of various supplements, as shown, and evaluated for immunomodulation by synthesis of indoleamine 2,3-dioxygenase (IDO) in response to cytokine stimulus. The samples are, left to right for each condition, FBS, Lysate A, Lysate C, Releasate F1, and Releasate F2.

FIG. 36 shows results from an immunomodulation experiment in which BM-MSCs were expanded in basal growth medium supplemented with one of various supplements, as shown, and evaluated for immunomodulation by co-culturing peripheral blood mononuclear cells (PBMCs) in the presence of the BM-MSCs for five days.

FIG. 37 shows results from an immunomodulation experiment in which BM-MSCs were expanded in basal growth medium supplemented with one of various supplements, as shown, and evaluated for immunomodulation by co-culturing PBMCs in the presence of the BM-MSCs for five days.

### DESCRIPTION OF ILLUSTRATIVE EMBODIMENTS

### I. Definitions

The term “Current Good Manufacturing Practice (cGMP)” as used herein refers to a system of minimum guidelines that ensures that products are consistently produced and controlled according to quality standards. It is designed to minimize the risks involved in any pharmaceutical production that cannot be eliminated through testing the final product. These guidelines provide minimum requirements that a manufacturer must meet to assure that their products are consistently high in quality, from batch to batch, for their intended use.

The term “cell culture” refers to the maintenance and propagation of cells in vitro. The cells may include stem cells and progenitor cells.

“Cell culture medium” is used for the maintenance of cells in culture in vitro. For some cell types, the medium may also be sufficient to support the expansion and proliferation of the cells in culture. A medium according to the present invention provides nutrients such as energy sources, amino acids and inorganic ions and other compounds known to a person of skill in the art,

The term “cell culture medium supplement” within the meaning of the present disclosure refers to a medium additive which is added to the medium to stimulate the proliferation, differentiation and expansion of the cells. Usually this supplement will contain one or more growth factors which are responsible for the stimulation of proliferation and/or differentiation.

The term “expansion of cells” is intended to mean the multiplication of cells thereby leading to an increase in the cell number.

The term “growth factor” is intended to comprise proteins which stimulate proliferation of cells by binding to a specific receptor. Usually, growth factors only act on specific cell types which express the respective receptor.

A “subject”, “individual” or “patient” or “casualty” is used interchangeably herein and refers to a vertebrate, for example a primate, a mammal, or a human. Mammals include, but are not limited to equines, canines, felines, porcines, bovines, chickens, felines, porcines, rabbits, dolphins, ovines, murines, rats, simians, humans, farm animals, sport animals, and pets. Also intended to be included herein as a subject any subjects involved in clinical research trials not showing any clinical sign of disease or subjects used as control.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. “Consisting essentially of” in the context of biologic compositions of the disclosure is intended to include all the recited active agents and excludes any additional non-recited active agents, but does not exclude other components of the composition that are not active ingredients. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this disclosure.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The use of the word “a” or “an” when used in conjunction with the term “comprising” in the claims and/or the specification may mean “one,” but it is also consistent with the meaning of “one or more,” “at least one,” and “one or more than one.”

The term “mesenchymal stromal cells” refers to the subpopulation of fibroblast or fibroblast-like nonhematopoietic cells with properties of plastic adherence and capable of in vitro differentiation into cells of mesodermal origin. Mesenchymal stromal cells be derived from bone marrow, adipose tissue, umbilical cord (Wharton's jelly), umbilical cord perivascular cells, umbilical cord blood, amniotic fluid, placenta, skin, dental pulp, breast milk, and synovial membrane. Mesenchymal stromal cells have a clonogenic capacity and can differentiate into several cells of mesodermal origin, such as adipocytes, osteoblasts, chondrocytes, skeletal myocytes, or visceral stromal cells. The term, “mesenchymal stem cells” refers to the cultured (self-renewed) progeny of primary mesenchymal stromal cell populations. Mesenchymal stromal/stem cells (MSCs) refers to mesenchymal stromal and/or mesenchymal stem cells.

The term “osteobiologic material” refers to any material that can induce and/or support existing or new bone growth. In some embodiments, the osteobiologic material is a load-bearing material. The term also includes engineered material (e.g., amnion-derived or placenta-derived materials, 3D printed constructs, etc.) that promote healing of fractures and bone defects.

A microcarrier is a support matrix that supports growth of adherent cells. Microcarrier cell culture may be carried out in flasks or bioreactors (e.g., hollow fiber bioreactors, wave bioreactors, etc.).

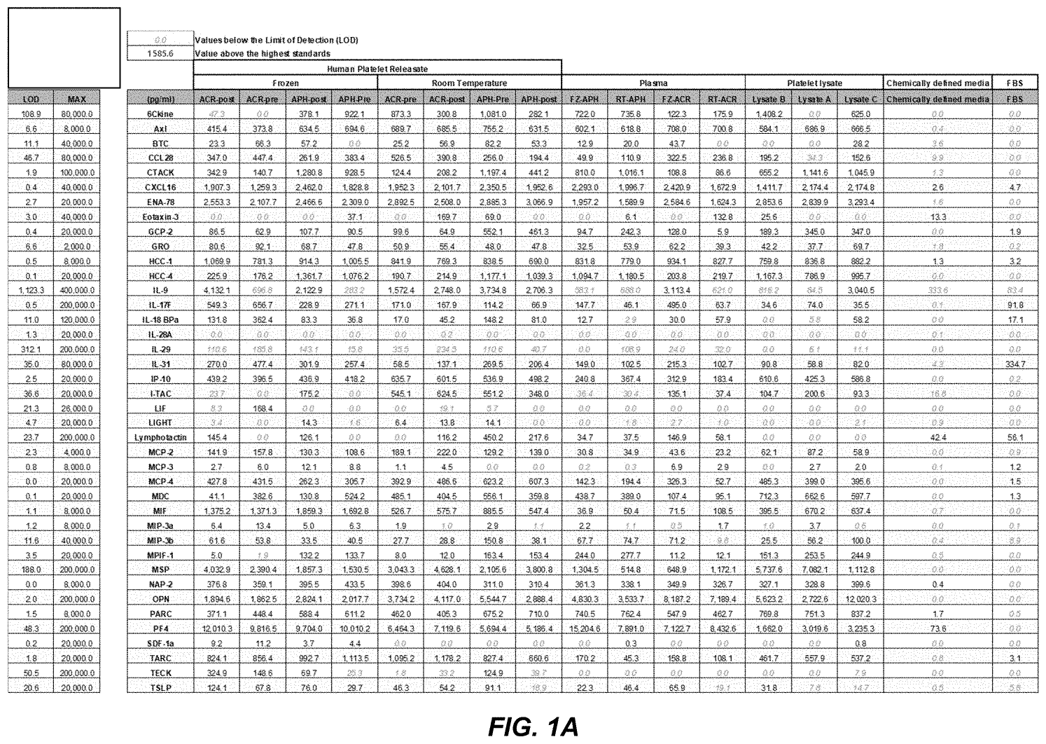

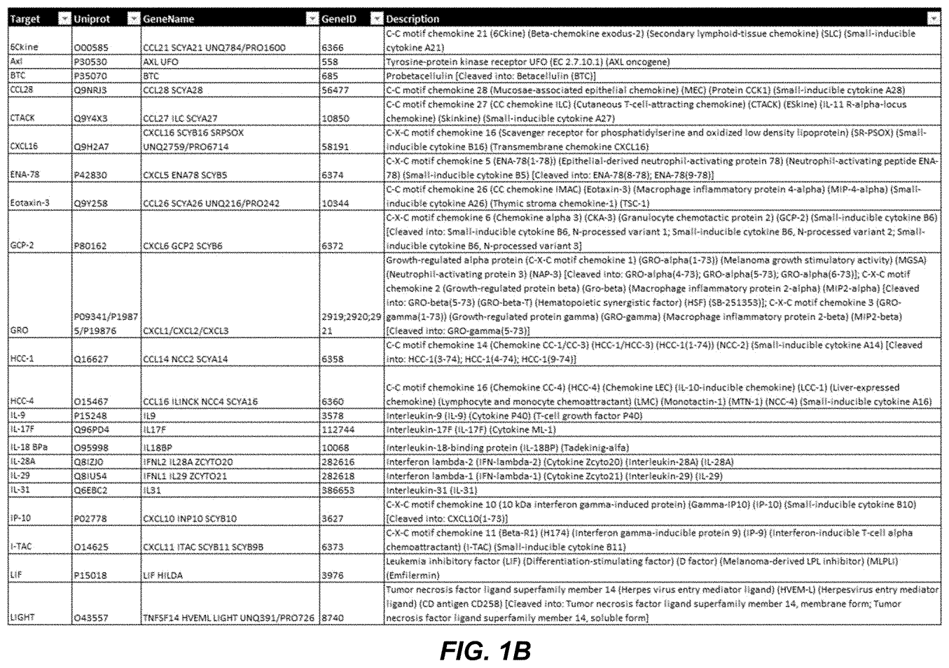

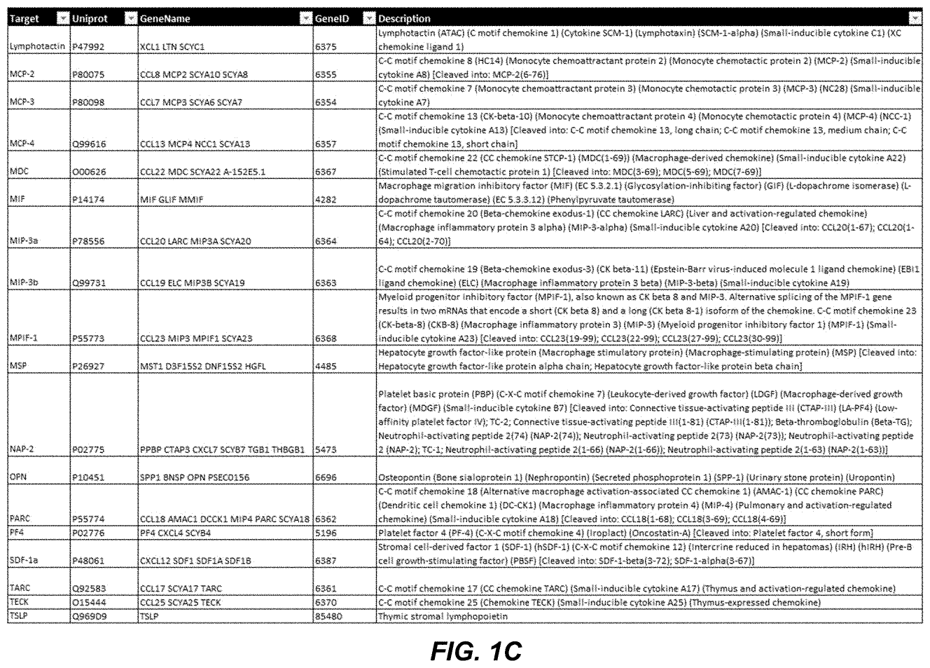

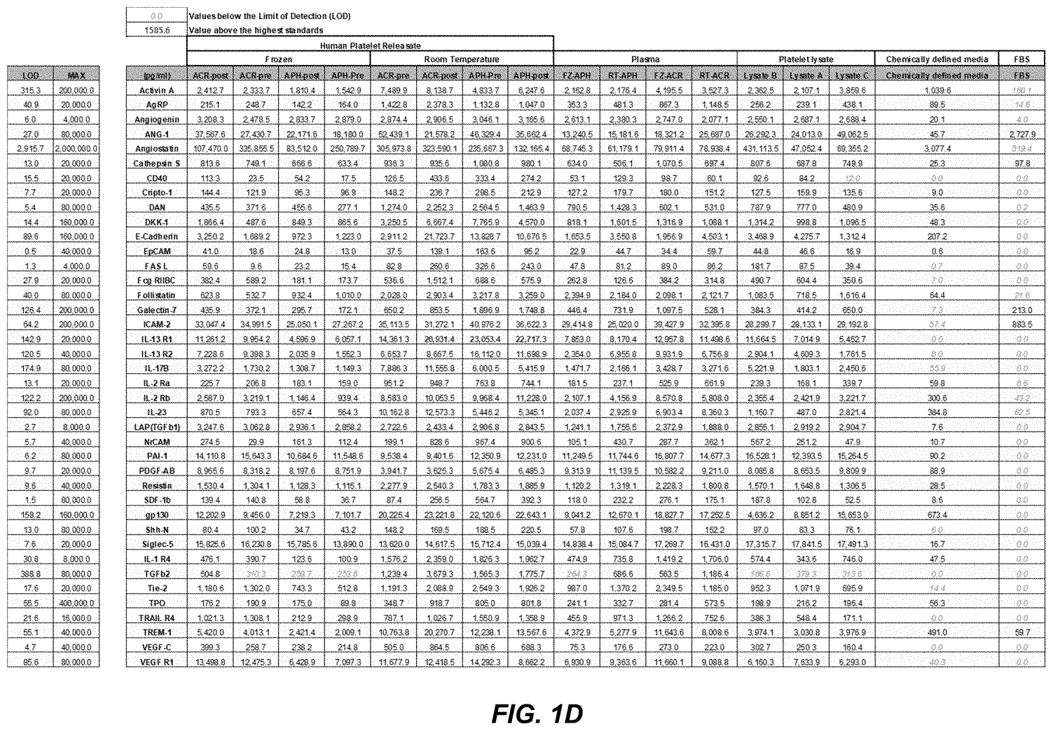

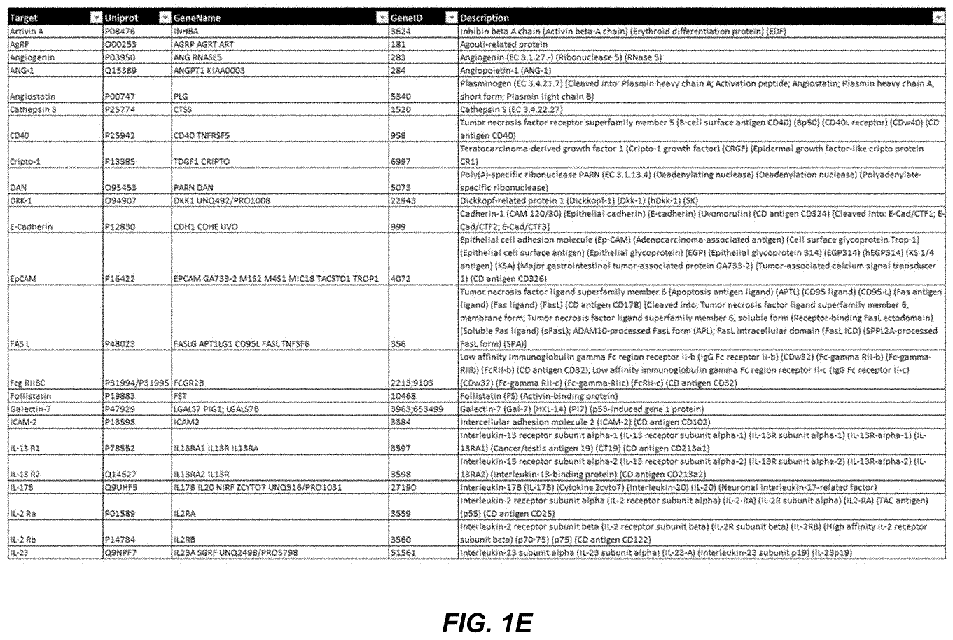

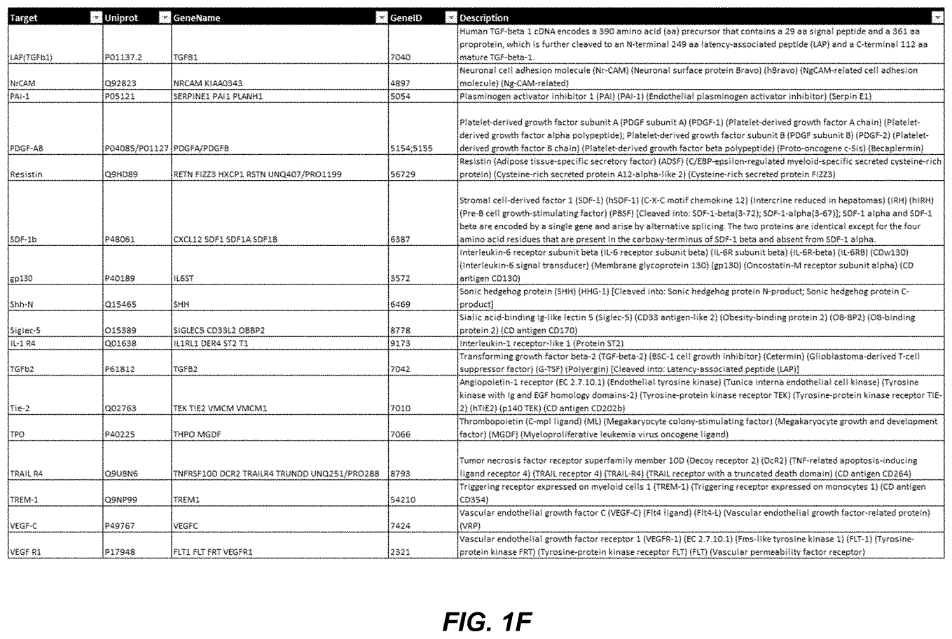

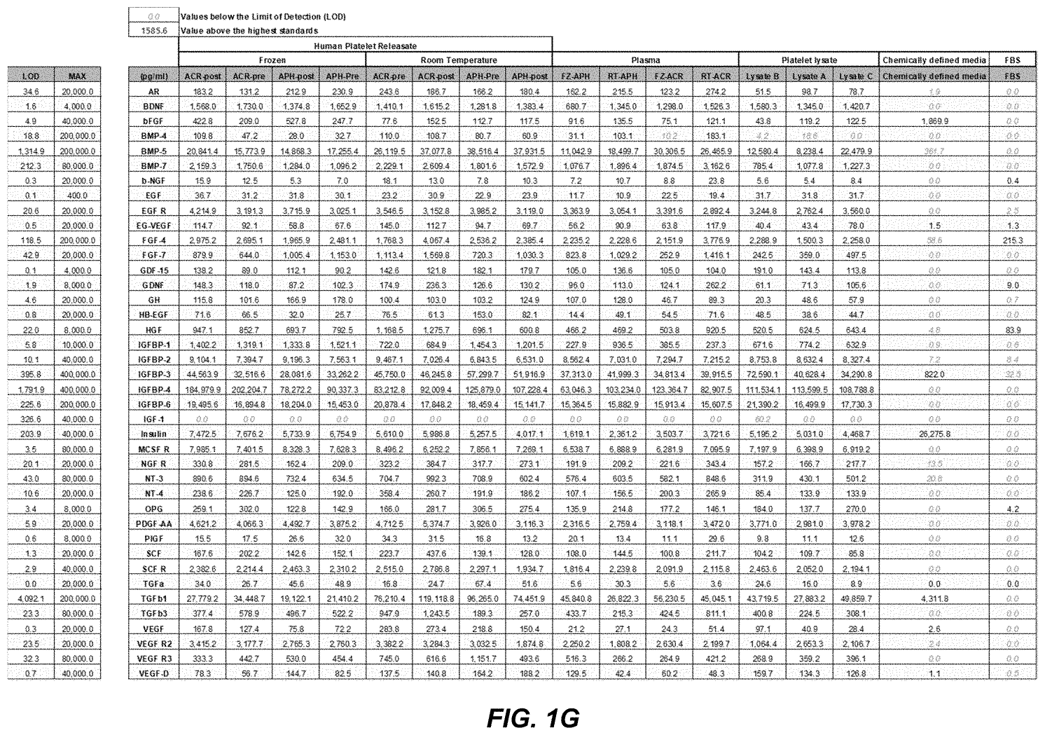

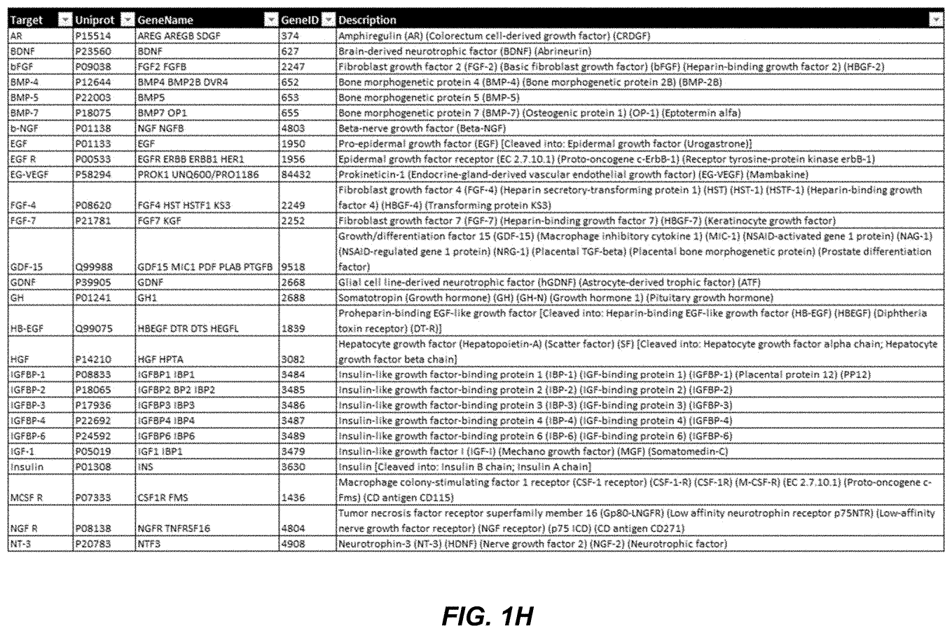

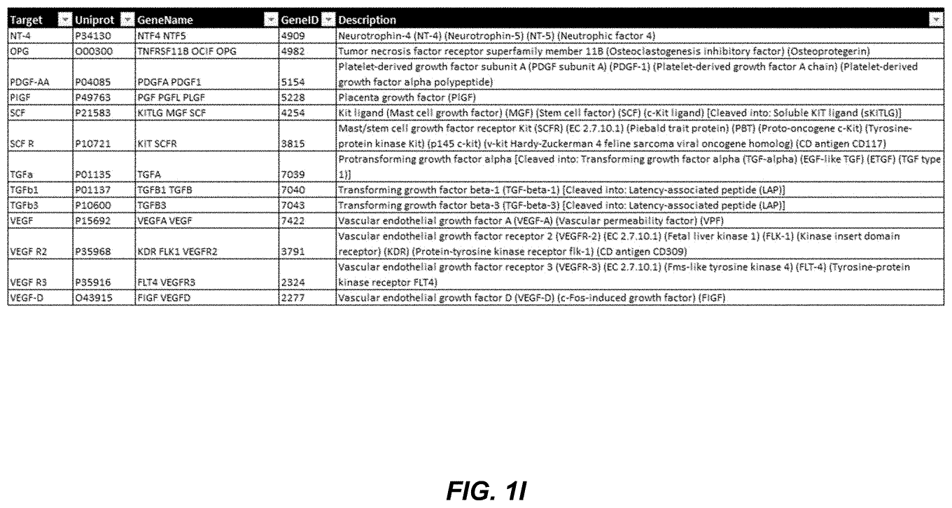

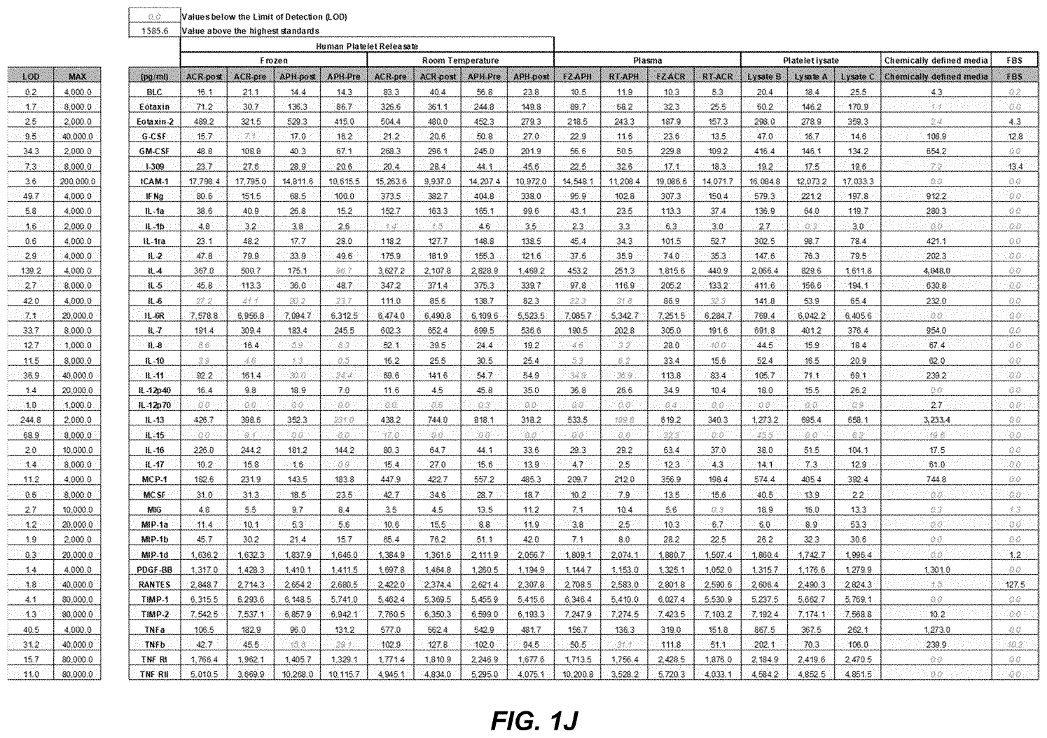

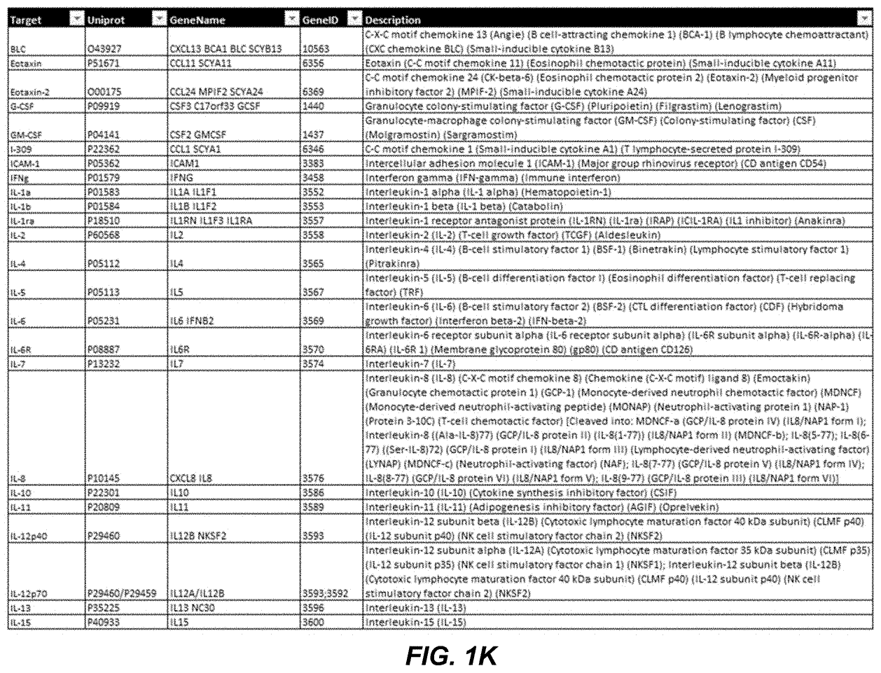

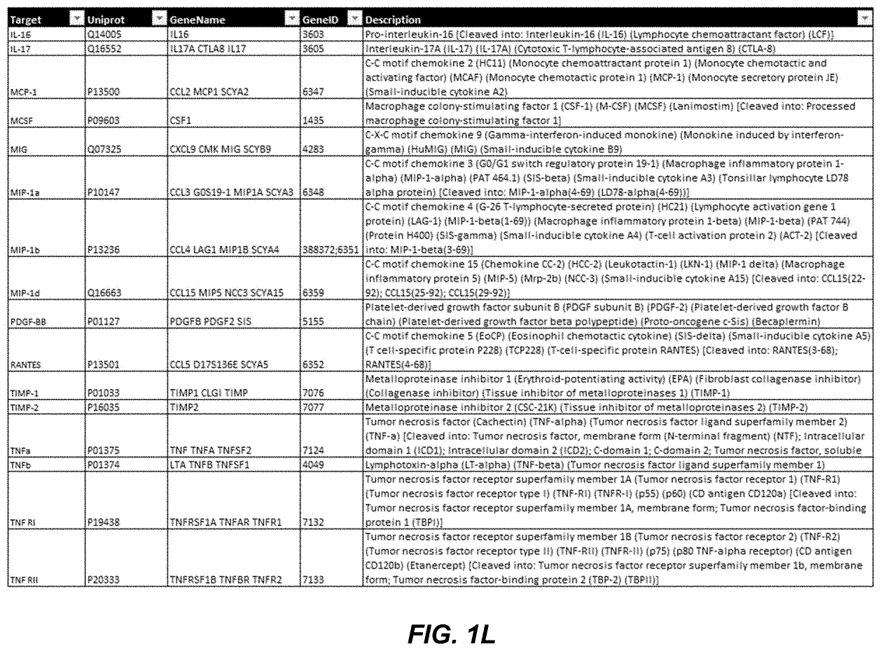

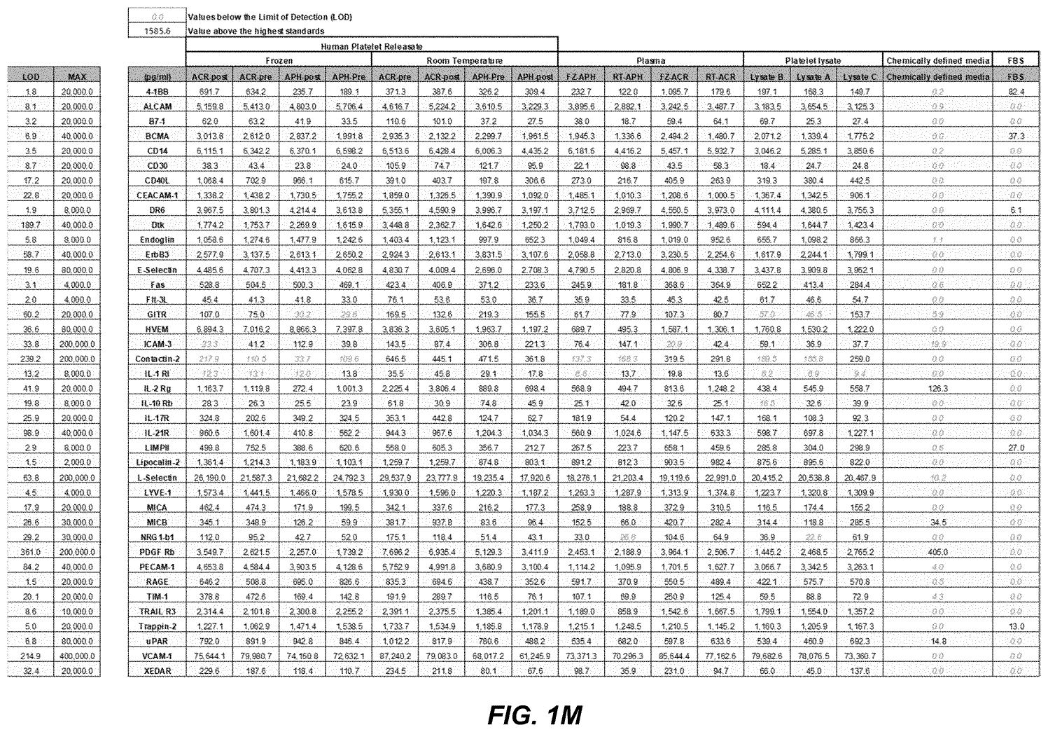

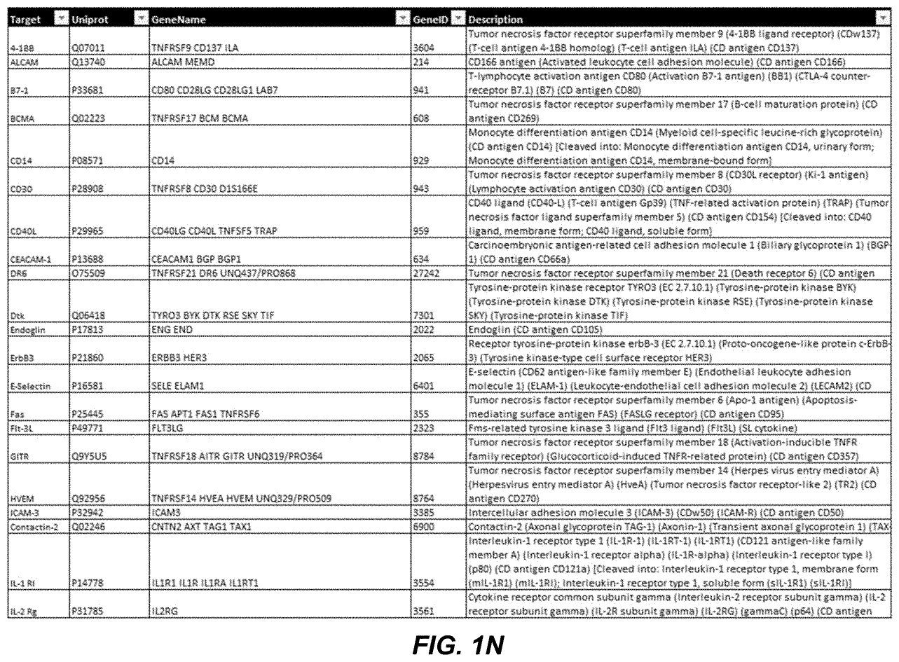

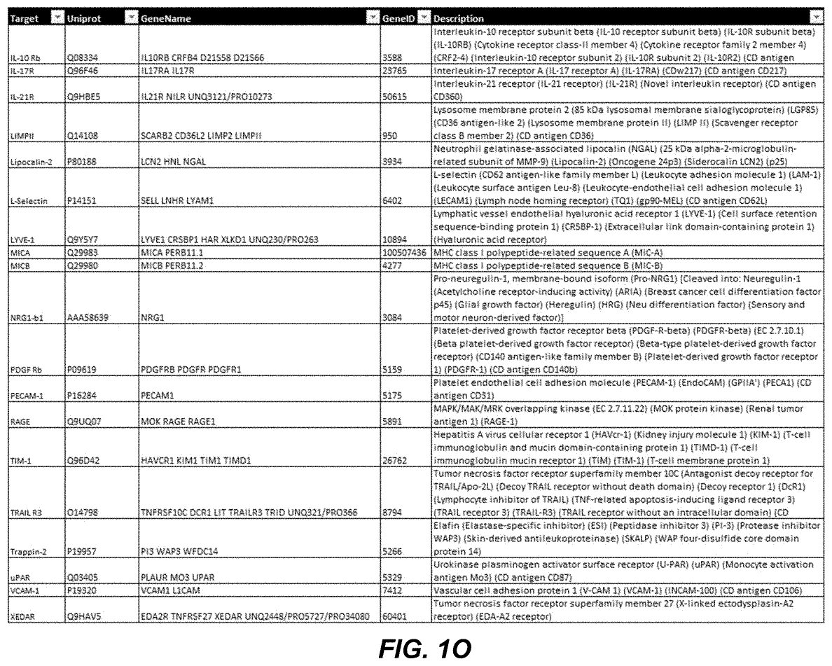

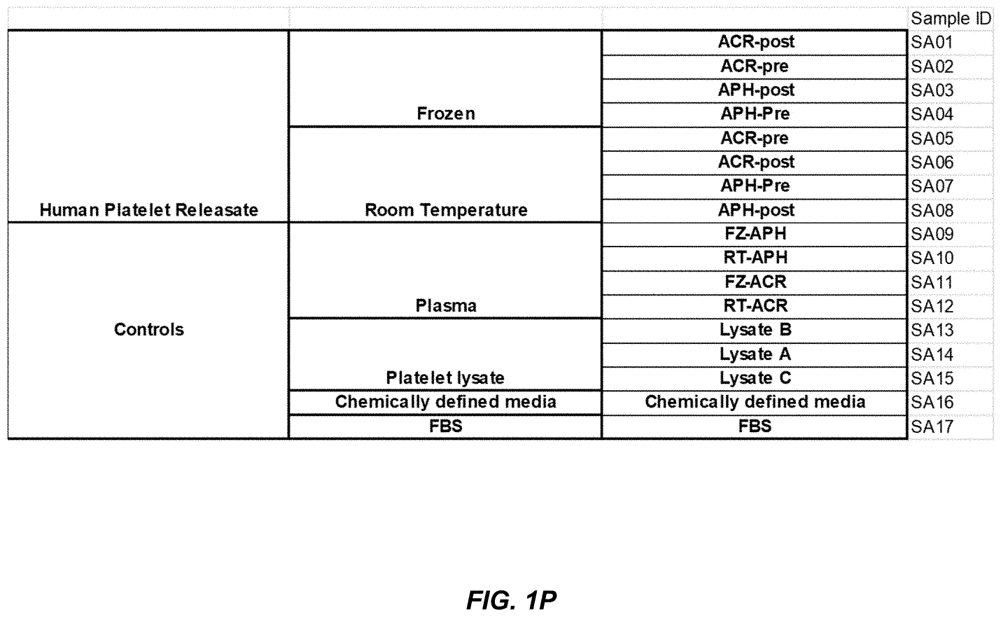

A “releasate”, in accordance with the current disclosure, is a product or extract obtained from cells when manipulated (e.g., degranulated) according to the protocols or methods of the current disclosure. A releasate describes a product obtained from cells in the absence of complete cellular lysis. A releasate may be obtained from platelet cells (i.e., may be a platelet releasate). The releasate can be used as a supplement in cell culture media to culture or expand cells. It can also be used in therapeutic applications including osteobiologics. It can include all of the growth factors or compounds in FIGS. 1A-1O or Table 1. It can exclude any one or any combination of the growth factors or compounds in FIGS. 1A-1O or Table 1. In some embodiments, a releasate comprises microvesicles or exosomes from the cells. In some embodiments, a releasate comprises exosomes isolated or purified from cells following a manipulation (e.g., degranulation) procedure as disclosed herein.

The terms “degranulate,” “degranulated,” “degranulating,” and “degranulation” describe protocols or methods for cellular manipulation disclosed herein, whereby one or more vesicles or intracellular compartments of a cell 1) release their contents outside the cell and/or 2) are removed or eliminated from the cell. Degranulation describes a process which does not comprise complete cell lysis.

As used herein, “treating,” “treatment,” or “therapy” is an approach or methodology used for obtaining beneficial or desired clinical results. This includes: the alleviation of symptoms, the reduction of inflammation, the amelioration of a diseased or wounded tissue or organ.

The term “stem cells” refers to any cell having the characteristic of being unspecialized and able to renew for extended periods of time through cell division and being inducible to become cells with specialized function.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive “or”.

Throughout this application, the term “about” is used to indicate that a value includes the inherent variation of error for the measurement or quantitation method.

### II. Overview

The inventors have developed process capable of industrially manufacturing platelet releasate (hPR)-a xeno-free alternative to both FBS and platelet lysate. hPR prevents gelation of growth medium, alleviating the need for heparin or other anticoagulants. The process of the current disclosure results in batches of hPR that are consistently replete with growth factors and other compounds beneficial for cell growth. In some embodiments, the hPR manufacturing process is carried on an industrial-scale, in a closed system that can yield up to 10 liters, 50 L, 100 L or hundreds of liters of hPR. In some embodiments the yield is 4.5-10 L. In some embodiments the yield is about, at least about, or at most about 2.0, 2.5, 3.0, 3.5, 4.5, 5.0, 5.5, 6.0, 7.0, 7.5, 8.0, 8.5, 9.0, 9.5, or 10 liters or any value derivable therein. In some embodiments the yield is about, at least about, or at most about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 liters or any value derivable therein. In some embodiments, a batch of 10, 20, 30, 40, 50 or 100 liters of hPR, for example, is prepared in a 10, 20, 30, 40, 50 or 100 L bag. In some embodiments, yields can be pooled.

The releasate of the current disclosure may be used a cell culture media supplement for culturing, expanding or differentiating a variety of cells including stem cells and their associated lineages. In some embodiments, releasate is used as a component of storage media for long term storage of cells, such as cold storage or cryo-storage. Example cells include, but are not limited, to pluripotent stem cells (PSC), induced pluripotent stem cells (iPSC), bone-marrow-derived-mesenchymal stromal/stem cells (BM-MSC), hematopoietic stem cells (HSC), Adipose-derived Mesenchymal Stromal/stem Cells (ADP-MSC), T-Cells, B-cells, Natural Killer cells, Dendritic cells, Peripheral Blood-derived Mononuclear Cells, cancer cells cancer stem cells, umbilical cord-blood-derived cells, umbilical cord-blood tissue-derived cells, placenta-derived cells, retinal cells, neurons, fibroblasts, epithelial cells, endothelial cells or keratinocytes. In addition, the releasate of the current disclosure may be used to culture/expand Chinese Hamster Ovarian (CHO) to manufacture antibodies, recombinant proteins and peptides. Other cells include fibroblasts, osteoblasts, adipocytes, chondrocytes endothelial cells, cells of the immune system, T-cells, B-cells, NK cells, engineered/manipulated cells and neurons.

Activation with Salt

Aspects of the disclosed methods comprise activation of platelets using a salt. The salt may act as a clotting agent when added to platelets causing the formation of a fibrin clot that is gel like and includes cellular debris. Various salts are contemplated and may be used in the disclosed methods for preparing releasate including, for example, calcium salts and magnesium salts. In some embodiments, calcium salts are used, for example calcium sulfate, calcium gluconate, calcium citrate, calcium phosphate, calcium chloride (CaCl₂)), calcium acetate, and/or calcium carbonate. In some embodiments, calcium chloride (CaCl₂)) is used. In some embodiments, magnesium salts are used, for example magnesium sulfate (MgSO4), magnesium gluconate, magnesium citrate, magnesium phosphate, magnesium chloride (MgCl₂), magnesium acetate, and/or magnesium carbonate.

In some aspects, the inventor has developed methods for preparing hPR from platelet-rich plasma that are based on the unexpected discovery that much higher salt concentrations, e.g., in the order of 25-80 mM for CaCl₂), results in an improved product. This discovery is contradictory to conventional recommendations and to what is taught in the art and leads to a much more efficient process that can be completed in 3-4 hours from start to finish. Upon removal of the clot, the resultant liquid is a clear supernatant that is replete with growth factors, microparticles (e.g., exosomes), and other bioactive substances. The amount of CaCl₂) that is added can result in a final concentration of CaCl₂) of about, at least about, or at most about 25, 30, 35, 40, 45, 50, 55, 60, 65, 70, 75, 80, 85, 90, 95, or 100 mM, or any other value derivable therein.

The source of platelets can be expired plasma-rich platelet or any suitable source of blood. In some embodiments, the source of blood is human. The source of platelets may be frozen or maintained at room temperature. The platelets may be frozen at −50° C. or at −80° C. or any suitable temperature in between. The platelets may be frozen for at least or at most 1, 2, 3, 4, 5, 6, 7, 8, 9, 10, 12, 18, or 24 hours, or any range or value derivable therein. In some embodiments, the platelets are frozen for less than 24 hours. The platelets may be frozen for at least or at most 1, 2, 3, 4, 5, 6, 7, 8, 9, 10, 15, 20, 30, 50, 100, 200, or 300 days, or any range or value derivable therein.

Expired units of platelet-rich plasma may be pooled as to ABO blood type. The blood cell count and/or infectious disease marker test results may be obtained and the units may be pooled together accordingly. In some embodiments, A-type plasma rich platelets are used. In some embodiments, B-type plasma rich platelets are used. In some embodiments, AB-type plasma rich platelets are used. In some embodiments, only O-type plasma rich platelets are used. In some aspects, platelets may be pooled to a volume of 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milliliters (ml), deciliters (dl) or liters (1 or L) including all values and ranges there between.

In some embodiments of the methods of the current disclosure, the platelets are concentrated prior to further processing. In some embodiments, the platelets are not concentrated prior to further processing. After adding the salt (e.g., CaCl₂)), to platelets, the mixture is agitated by any suitable method such as for example using an orbital shaker to agitate the contents. The agitation can be done at any suitable rpm. In some embodiments, the CaCl₂/PRP mixture is agitated at 50-500 rpm The agitation can be done at any suitable rpm. In some embodiments, the CaCl₂/PRP mixture is agitated at 150-350 rpm. In some embodiments, the CaCl₂/PRP mixture is agitated at, at least, or at most about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500 rpm or any value derivable therein. In some embodiments of the methods of the current disclosure that comprise batch sizes of 50 or 100 L, the rpm may be, be at least, or be at most 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 215, 220, 225, 230, 235, 240, 245, 250, or any value derivable therein. Agitation may comprise one or more mechanical forces. In some embodiments, agitation is severe agitation and comprises severe mechanical forces. In some embodiments, agitation is mild agitation and comprises mild mechanical forces. Mechanical forces which may be used for agitation include, for example, grinding (e.g. mortar/pestle, cryogrinders, glass homogenizers, bead homogenizers), beating using beads and/or paddles, shearing (e.g., blending; vortex with glass, ceramic or steel beads; homogenization with rotor/stator, dounce homogenizer), acoustics and ultrasonication (e.g., bath and probe sonicators; infrasonic, sonic and ultrasonic wavelengths), french press/french pressure cell, optical methods (e.g., thermolysis, cavitation), electromagnetic field (e.g., microwave, shocking, electroporation), rocking, shaking (e.g., vortex shaker, platform shaker, orbital shaker, incubator shaker), stirring (e.g., magnetic stirring), fluidization, milling, freezing/thermal treatment, and high pressure homogenization.

After the addition of the salt (e.g., CaCl₂)), the clotting time may be any suitable time period and the mixture may be agitated. In some embodiments of the methods of the current disclosure, the CaCl₂/PRP mixture is allowed to form a clot for 20 min-4 hours. In some embodiments, the clotting time is between 60 min-90 min. The clotting time may be 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240 minutes or any suitable amount in between. In some embodiments, the CaCl₂/PRP mixture is agitated for less than 180 minutes. In further embodiments, the CaCl₂/PRP mixture is agitated for 30-150 minutes or from 45-135 minutes. Yet in other embodiments, the CaCl₂/PRP mixture is agitated from 60-90 minutes. In some embodiments, some agents (such as Kaolin) can expedite clotting in 2-5 mins. It is contemplated that when using such agents with the current methods, the clotting time may be, be at least, or be at most 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240 minutes or any range derivable therein.

After a clot is formed, it can be separated from the liquid material by any suitable method. In one embodiment, the clot is separated by centrifugation. In some the mixture is centrifuged at a suitable temperature. For example, the mixture can be centrifuged at 4000 g at 4° C. In one embodiment, the clot is separated from the liquid material by decanting the liquid using an expressor or gravity separation techniques. It can be centrifuged for, for at least, or for at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 59, 60 minutes and/or 1, 2, 3, 4 or more hours (or any range derivable therein) at about, at least about, or at most about 500, 1000, 2000, 3000, 4000, 5000, 6000, 7000, 8000 g or more (or any range derivable therein).

In some embodiments, the methods of the current disclosure further comprise the step of filtering the releasate. Filtering the releasate decreases the amount of fibrinogen in the final product as well as other contaminants. Filtration can be done in one or double filtration. In some embodiments, the releasate is filtered using a filter that is between 0.45-1.0 microns. In some embodiments, the releasate is filtered using a filter that is between 0.45-0.65 microns. In some embodiments, the filter is 0.45, 0.50, 0.55, 0.60, 0.65, 0.70, 0.75, 0.80, 0.85, 0.90, 0.95, 1.0 microns or any size derivable therein. In some embodiments, the filter is a 3-10 micron filter. In some embodiments the filter is, is at least, or is at most 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0 microns or any size derivable therein. In some embodiments, the filter is a 170-260 micron filter. In some embodiments the filter is 170, 175, 180, 185, 190, 195, 200, 205, 210, 215, 220, 225, 230, 235, 240, 245, 250, 255, 260 micron filter or any range derivable therein.

In some embodiments, releasate is pooled following generation. In some aspects, releasate may be pooled to a volume of 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milliliters (ml), deciliters (dl) or liters (I or L) including all values and ranges there between.

Growth Factors and Other Bioactive Compounds in hPR

The hPR product according to the current disclosure retains multiple bioactive components including growth factors, cytokines, and chemokines in such amounts and/or combinations that stem cells, such as bone marrow-derived mesenchymal stem cells, when expanded in the presence of hPR have superior expansion rates compared to FBS, platelet lysates, and chemically-defined media. The growth factors, cytokines, chemokines, interferons or other components may be any combination or all of: Amphiregulin (AR) (Colorectum cell-derived growth factor) (CRDGF), Brain-derived neurotrophic factor (BDNF) (Abrineurin), Fibroblast growth factor 2 (FGF-2) (Basic fibroblast growth factor) (bFGF) (Heparin-binding growth factor 2) (HBGF-2), Bone morphogenetic protein 4 (BMP-4) (Bone morphogenetic protein 2B) (BMP-2B), Bone morphogenetic protein 5 (BMP-5), Bone morphogenetic protein 7 (BMP-7) (Osteogenic protein 1) (OP-1) (Eptotermin alfa), Beta-nerve growth factor (Beta-NGF), Pro-epidermal growth factor (EGF) [Cleaved into: Epidermal growth factor (Urogastrone)], Epidermal growth factor receptor (EC 2.7.10.1) (Proto-oncogene c-ErbB-1) (Receptor tyrosine-protein kinase erbB-1), Prokineticin-1 (Endocrine-gland-derived vascular endothelial growth factor) (EG-VEGF) (Mambakine), Fibroblast growth factor 4 (FGF-4) (Heparin secretory-transforming protein 1) (HST) (HST-1) (HSTF-1) (Heparin-binding growth factor 4) (HBGF-4) (Transforming protein KS3), Fibroblast growth factor 7 (FGF-7) (Heparin-binding growth factor 7) (HBGF-7) (Keratinocyte growth factor), Growth/differentiation factor 15 (GDF-15) (Macrophage inhibitory cytokine 1) (MIC-1) (NSAID-activated gene 1 protein) (NAG-1) (NSAID-regulated gene 1 protein) (NRG-1) (Placental TGF-beta) (Placental bone morphogenetic protein) (Prostate differentiation factor), Glial cell line-derived neurotrophic factor (hGDNF) (Astrocyte-derived trophic factor) (ATF), Somatotropin (Growth hormone) (GH) (GH-N) (Growth hormone 1) (Pituitary growth hormone), Proheparin-binding EGF-like growth factor [Cleaved into: Heparin-binding EGF-like growth factor (HB-EGF) (HBEGF) (Diphtheria toxin receptor) (DT-R)], Hepatocyte growth factor (Hepatopoietin-A) (Scatter factor) (SF) [Cleaved into: Hepatocyte growth factor alpha chain; Hepatocyte growth factor beta chain], Insulin-like growth factor-binding protein 1 (IBP-1) (IGF-binding protein 1) (IGFBP-1) (Placental protein 12) (PP12), Insulin-like growth factor-binding protein 2 (IBP-2) (IGF-binding protein 2) (IGFBP-2), Insulin-like growth factor-binding protein 3 (IBP-3) (IGF-binding protein 3) (IGFBP-3), Insulin-like growth factor-binding protein 4 (IBP-4) (IGF-binding protein 4) (IGFBP-4), Insulin-like growth factor-binding protein 6 (IBP-6) (IGF-binding protein 6) (IGFBP-6), Insulin-like growth factor I (IGF-I) (Mechano growth factor) (MGF) (Somatomedin-C), Insulin [Cleaved into: Insulin B chain; Insulin A chain], Macrophage colony-stimulating factor 1 receptor (CSF-1 receptor) (CSF-1-R) (CSF-1R) (M-CSF-R) (EC 2.7.10.1) (Proto-oncogene c-Fms) (CD antigen CD115), Tumor necrosis factor receptor superfamily member 16 (Gp80-LNGFR) (Low affinity neurotrophin receptor p75NTR) (Low-affinity nerve growth factor receptor) (NGF receptor) (p75 ICD) (CD antigen CD271), Neurotrophin-3 (NT-3) (HDNF) (Nerve growth factor 2) (NGF-2) (Neurotrophic factor), Neurotrophin-4 (NT-4) (Neurotrophin-5) (NT-5) (Neutrophic factor 4), Tumor necrosis factor receptor superfamily member 11B (Osteoclastogenesis inhibitory factor) (Osteoprotegerin), Platelet-derived growth factor subunit A (PDGF subunit A) (PDGF-1) (Platelet-derived growth factor A chain) (Platelet-derived growth factor alpha polypeptide), Placenta growth factor (PlGF), Kit ligand (Mast cell growth factor) (MGF) (Stem cell factor) (SCF) (c-Kit ligand) [Cleaved into: Soluble KIT ligand (sKITLG)], Mast/stem cell growth factor receptor Kit (SCFR) (EC 2.7.10.1) (Piebald trait protein) (PBT) (Proto-oncogene c-Kit) (Tyrosine-protein kinase Kit) (p145 c-kit) (v-kit Hardy-Zuckerman 4 feline sarcoma viral oncogene homolog) (CD antigen CD117), Protransforming growth factor alpha [Cleaved into: Transforming growth factor alpha (TGF-alpha) (EGF-like TGF) (ETGF) (TGF type 1)], Transforming growth factor beta-1 (TGF-beta-1) [Cleaved into: Latency-associated peptide (LAP)], Transforming growth factor beta-3 (TGF-beta-3) [Cleaved into: Latency-associated peptide (LAP)], Vascular endothelial growth factor A (VEGF-A) (Vascular permeability factor) (VPF), Vascular endothelial growth factor receptor 2 (VEGFR-2) (EC 2.7.10.1) (Fetal liver kinase 1) (FLK-1) (Kinase insert domain receptor) (KDR) (Protein-tyrosine kinase receptor flk-1) (CD antigen CD309), Vascular endothelial growth factor receptor 3 (VEGFR-3) (EC 2.7.10.1) (Fms-like tyrosine kinase 4) (FLT-4) (Tyrosine-protein kinase receptor FLT4), Vascular endothelial growth factor D (VEGF-D) (c-Fos-induced growth factor) (FIGF). In some embodiments, it is specifically contemplated that any of these may be included in a yield, but in other embodiments, it is specifically contemplated that any of these may not be included in a yield.

Other bioactive compounds include: C—C motif chemokine 21 (6Ckine) (Beta-chemokine exodus-2) (Secondary lymphoid-tissue chemokine) (SLC) (Small-inducible cytokine A21), Tyrosine-protein kinase receptor UFO (EC 2.7.10.1) (AXL oncogene), Probetacellulin [Cleaved into: Betacellulin (BTC)], C—C motif chemokine 28 (Mucosae-associated epithelial chemokine) (MEC) (Protein CCK1) (Small-inducible cytokine A28), C—C motif chemokine 27 (CC chemokine ILC) (Cutaneous T-cell-attracting chemokine) (CTACK) (ESkine) (IL-11 R-alpha-locus chemokine) (Skinkine) (Small-inducible cytokine A27), C—X—C motif chemokine 16 (Scavenger receptor for phosphatidylserine and oxidized low density lipoprotein) (SR-PSOX) (Small-inducible cytokine B16) (Transmembrane chemokine CXCL16), C—X—C motif chemokine 5 (ENA-78(1-78)) (Epithelial-derived neutrophil-activating protein 78) (Neutrophil-activating peptide ENA-78) (Small-inducible cytokine B5) [Cleaved into: ENA-78(8-78); ENA-78(9-78)], C—C motif chemokine 26 (CC chemokine IMAC) (Eotaxin-3) (Macrophage inflammatory protein 4-alpha) (MIP-4-alpha) (Small-inducible cytokine A26) (Thymic stroma chemokine-1) (TSC-1), C—X—C motif chemokine 6 (Chemokine alpha 3) (CKA-3) (Granulocyte chemotactic protein 2) (GCP-2) (Small-inducible cytokine B6) [Cleaved into: Small-inducible cytokine B6, N-processed variant 1; Small-inducible cytokine B6, N-processed variant 2; Small-inducible cytokine B6, N-processed variant 3], Growth-regulated alpha protein (C—X—C motif chemokine 1) (GRO-alpha(1-73)) (Melanoma growth stimulatory activity) (MGSA) (Neutrophil-activating protein 3) (NAP-3) [Cleaved into: GRO-alpha(4-73); GRO-alpha(5-73); GRO-alpha(6-73)]; C—X—C motif chemokine 2 (Growth-regulated protein beta) (Gro-beta) (Macrophage inflammatory protein 2-alpha) (MIP2-alpha) [Cleaved into: GRO-beta(5-73) (GRO-beta-T) (Hematopoietic synergistic factor) (HSF) (SB-251353)]; C—X—C motif chemokine 3 (GRO-gamma(1-73)) (Growth-regulated protein gamma) (GRO-gamma) (Macrophage inflammatory protein 2-beta) (MIP2-beta) [Cleaved into: GRO-gamma(5-73)], C—C motif chemokine 14 (Chemokine CC-1/CC-3) (HCC-1/HCC-3) (HCC-1(1-74)) (NCC-2) (Small-inducible cytokine A14) [Cleaved into: HCC-1(3-74); HCC-1(4-74); HCC-1(9-74)], C—C motif chemokine 16 (Chemokine CC-4) (HCC-4) (Chemokine LEC) (IL-10-inducible chemokine) (LCC-1) (Liver-expressed chemokine) (Lymphocyte and monocyte chemoattractant) (LMC) (Monotactin-1) (MTN-1) (NCC-4) (Small-inducible cytokine A16), Interleukin-9 (IL-9) (Cytokine P40) (T-cell growth factor P40), Interleukin-17F (IL-17F) (Cytokine ML-1), Interleukin-18-binding protein (IL-18BP) (Tadekinig-alfa), Interferon lambda-2 (IFN-lambda-2) (Cytokine Zcyto20) (Interleukin-28A) (IL-28A), Interferon lambda-1 (IFN-lambda-1) (Cytokine Zcyto21) (Interleukin-29) (IL-29), Interleukin-31 (IL-31), C—X—C motif chemokine 10 (10 kDa interferon gamma-induced protein) (Gamma-IP10) (IP-10) (Small-inducible cytokine B10) [Cleaved into: CXCL10(1-73)], C—X—C motif chemokine 11 (Beta-R1) (H174) (Interferon gamma-inducible protein 9) (IP-9) (Interferon-inducible T-cell alpha chemoattractant) (I-TAC) (Small-inducible cytokine B11), Leukemia inhibitory factor (LIF) (Differentiation-stimulating factor) (D factor) (Melanoma-derived LPL inhibitor) (MLPLI) (Emfilermin), Tumor necrosis factor ligand superfamily member 14 (Herpes virus entry mediator ligand) (HVEM-L) (Herpesvirus entry mediator ligand) (CD antigen CD258) [Cleaved into: Tumor necrosis factor ligand superfamily member 14, membrane form; Tumor necrosis factor ligand superfamily member 14, soluble form], Lymphotactin (ATAC) (C motif chemokine 1) (Cytokine SCM-1) (Lymphotaxin) (SCM-1-alpha) (Small-inducible cytokine C1) (XC chemokine ligand 1), C—C motif chemokine 8 (HC14) (Monocyte chemoattractant protein 2) (Monocyte chemotactic protein 2) (MCP-2) (Small-inducible cytokine A8) [Cleaved into: MCP-2(6-76)], C—C motif chemokine 7 (Monocyte chemoattractant protein 3) (Monocyte chemotactic protein 3) (MCP-3) (NC28) (Small-inducible cytokine A7), C—C motif chemokine 13 (CK-beta-10) (Monocyte chemoattractant protein 4) (Monocyte chemotactic protein 4) (MCP-4) (NCC-1) (Small-inducible cytokine A13) [Cleaved into: C—C motif chemokine 13, long chain; C—C motif chemokine 13, medium chain; C—C motif chemokine 13, short chain], C—C motif chemokine 22 (CC chemokine STCP-1) (MDC(1-69)) (Macrophage-derived chemokine) (Small-inducible cytokine A22) (Stimulated T-cell chemotactic protein 1) [Cleaved into: MDC(3-69); MDC(5-69); MDC(7-69)] Macrophage migration inhibitory factor (MIF) (EC 5.3.2.1) (Glycosylation-inhibiting factor) (GIF) (L-dopachrome isomerase) (L-dopachrome tautomerase) (EC 5.3.3.12) (Phenylpyruvate tautomerase), C—C motif chemokine 20 (Beta-chemokine exodus-1) (CC chemokine LARC) (Liver and activation-regulated chemokine) (Macrophage inflammatory protein 3 alpha) (MIP-3-alpha) (Small-inducible cytokine A20) [Cleaved into: CCL20(1-67); CCL20(1-64); CCL20(2-70)], C—C motif chemokine 19 (Beta-chemokine exodus-3) (CKbeta-11) (Epstein-Barr virus-induced molecule 1 ligand chemokine) (EBIl ligand chemokine) (ELC) (Macrophage inflammatory protein 3 beta) (MIP-3-beta) (Small-inducible cytokine A19), Myeloid progenitor inhibitory factor (MPIF-1), also known as CK beta 8 and MIP-3. Alternative splicing of the MPIF-1 gene results in two mRNAs that encode a short (CK beta 8) and a long (CK beta 8-1) isoform of the chemokine. C—C motif chemokine 23 (CK-beta-8) (CKB-8) (Macrophage inflammatory protein 3) (MIP-3) (Myeloid progenitor inhibitory factor 1) (MPIF-1) (Small-inducible cytokine A23) [Cleaved into: CCL23(19-99); CCL23(22-99); CCL23(27-99); CCL23(30-99)], Hepatocyte growth factor-like protein (Macrophage stimulatory protein) (Macrophage-stimulating protein) (MSP) [Cleaved into: Hepatocyte growth factor-like protein alpha chain; Hepatocyte growth factor-like protein beta chain], Platelet basic protein (PBP) (C—X—C motif chemokine 7) (Leukocyte-derived growth factor) (LDGF) (Macrophage-derived growth factor) (MDGF) (Small-inducible cytokine B7) [Cleaved into: Connective tissue-activating peptide III (CTAP-III) (LA-PF4) (Low-affinity platelet factor IV); TC-2; Connective tissue-activating peptide III(1-81) (CTAP-III(1-81)); Beta-thromboglobulin (Beta-TG); Neutrophil-activating peptide 2(74) (NAP-2(74)); Neutrophil-activating peptide 2(73) (NAP-2(73)); Neutrophil-activating peptide 2 (NAP-2); TC-1; Neutrophil-activating peptide 2(1-66) (NAP-2(1-66)); Neutrophil-activating peptide 2(1-63) (NAP-2(1-63))], Osteopontin (Bone sialoprotein 1) (Nephropontin) (Secreted phosphoprotein 1) (SPP-1) (Urinary stone protein) (Uropontin), C—C motif chemokine 18 (Alternative macrophage activation-associated CC chemokine 1) (AMAC-1) (CC chemokine PARC) (Dendritic cell chemokine 1) (DC-CK1) (Macrophage inflammatory protein 4) (MIP-4) (Pulmonary and activation-regulated chemokine) (Small-inducible cytokine A18) [Cleaved into: CCL18(1-68); CCL18(3-69); CCL18(4-69)], Platelet factor 4 (PF-4) (C—X—C motif chemokine 4) (Iroplact) (Oncostatin-A) [Cleaved into: Platelet factor 4, short form], Stromal cell-derived factor 1 (SDF-1) (hSDF-1) (C—X—C motif chemokine 12) (Intercrine reduced in hepatomas) (IRH) (hIRH) (Pre-B cell growth-stimulating factor) (PBSF) [Cleaved into: SDF-1-beta(3-72); SDF-1α (3-67)], C—C motif chemokine 17 (CC chemokine TARC) (Small-inducible cytokine A17) (Thymus and activation-regulated chemokine), and C—C motif chemokine 25 (Chemokine TECK) (Small-inducible cytokine A25) (Thymus-expressed chemokine), Thymic stromal lymphopoietin. In some embodiments, it is specifically contemplated that any of these may be included in a yield, but in other embodiments, it is specifically contemplated that any of these may not be included in a yield.

Additional bioactive substances present in the releasate may include: Inhibin beta A chain (Activin beta-A chain) (Erythroid differentiation protein) (EDF), Agouti-related protein, Angiogenin (EC 3.1.27.-) (Ribonuclease 5) (RNase 5), Angiopoietin-1 (ANG-1), Plasminogen (EC 3.4.21.7) [Cleaved into: Plasmin heavy chain A; Activation peptide; Angiostatin; Plasmin heavy chain A, short form; Plasmin light chain B], Cathepsin S (EC 3.4.22.27), Tumor necrosis factor receptor superfamily member 5 (B-cell surface antigen CD40) (Bp50) (CD40 L receptor) (CDw40) (CD antigen CD40), Teratocarcinoma-derived growth factor 1 (Cripto-1 growth factor) (CRGF) (Epidermal growth factor-like cripto protein CR1), Poly(A)-specific ribonuclease PARN (EC 3.1.13.4) (Deadenylating nuclease) (Deadenylation nuclease) (Polyadenylate-specific ribonuclease), Dickkopf-related protein 1 (Dickkopf-1) (Dkk-1) (hDkk-1) (SK), Cadherin-1 (CAM 120/80) (Epithelial cadherin) (E-cadherin) (Uvomorulin) (CD antigen CD324) [Cleaved into: E-Cad/CTF1; E-Cad/CTF2; E-Cad/CTF3], Epithelial cell adhesion molecule (Ep-CAM) (Adenocarcinoma-associated antigen) (Cell surface glycoprotein Trop-1) (Epithelial cell surface antigen) (Epithelial glycoprotein) (EGP) (Epithelial glycoprotein 314) (EGP314) (hEGP314) (KS 1/4 antigen) (KSA) (Major gastrointestinal tumor-associated protein GA733-2) (Tumor-associated calcium signal transducer 1) (CD antigen CD326), Tumor necrosis factor ligand superfamily member 6 (Apoptosis antigen ligand) (APTL) (CD95 ligand) (CD95-L) (Fas antigen ligand) (Fas ligand) (FasL) (CD antigen CD178) [Cleaved into: Tumor necrosis factor ligand superfamily member 6, membrane form; Tumor necrosis factor ligand superfamily member 6, soluble form (Receptor-binding FasL ectodomain) (Soluble Fas ligand) (sFasL); ADAM10-processed FasL form (APL); FasL intracellular domain (FasL ICD) (SPPL2A-processed FasL form) (SPA)], Low affinity immunoglobulin gamma Fc region receptor II-b (IgG Fc receptor II-b) (CDw32) (Fc-gamma RII-b) (Fc-gamma-RIIb) (FcRII-b) (CD antigen CD32); Low affinity immunoglobulin gamma Fc region receptor II-c (IgG Fc receptor II-c) (CDw32) (Fc-gamma RII-c) (Fc-gamma-RIIc) (FcRII-c) (CD antigen CD32), Follistatin (FS) (Activin-binding protein), Galectin-7 (Gal-7) (HKL-14) (PI7) (p53-induced gene 1 protein), Intercellular adhesion molecule 2 (ICAM-2) (CD antigen CD102), Interleukin-13 receptor subunit alpha-1 (IL-13 receptor subunit alpha-1) (IL-13R subunit alpha-1) (IL-13R-alpha-1) (IL-13RA1) (Cancer/testis antigen 19) (CT19) (CD antigen CD213a1), Interleukin-13 receptor subunit alpha-2 (IL-13 receptor subunit alpha-2) (IL-13R subunit alpha-2) (IL-13R-alpha-2) (IL-13RA2) (Interleukin-13-binding protein) (CD antigen CD213a2), Interleukin-17B (IL-17B) (Cytokine Zcyto7) (Interleukin-20) (IL-20) (Neuronal interleukin-17-related factor), Interleukin-2 receptor subunit alpha (IL-2 receptor subunit alpha) (IL-2-RA) (IL-2R subunit alpha) (IL2-RA) (TAC antigen) (p55) (CD antigen CD25), Interleukin-2 receptor subunit beta (IL-2 receptor subunit beta) (IL-2R subunit beta) (IL-2RB) (High affinity IL-2 receptor subunit beta) (p70-75) (p75) (CD antigen CD122), Interleukin-23 subunit alpha (IL-23 subunit alpha) (IL-23-A) (Interleukin-23 subunit p19) (IL-23p19), Human TGF-beta 1 cDNA encodes a 390 amino acid (aa) precursor that contains a 29 aa signal peptide and a 361 aa proprotein, which is further cleaved to an N-terminal 249 aa latency-associated peptide (LAP) and a C-terminal 112 aa mature TGF-beta-1, Neuronal cell adhesion molecule (Nr-CAM) (Neuronal surface protein Bravo) (hBravo) (NgCAM-related cell adhesion molecule) (Ng-CAM-related), Plasminogen activator inhibitor 1 (PAI) (PAI-1) (Endothelial plasminogen activator inhibitor) (Serpin E1), Platelet-derived growth factor subunit A (PDGF subunit A) (PDGF-1) (Platelet-derived growth factor A chain) (Platelet-derived growth factor alpha polypeptide); Platelet-derived growth factor subunit B (PDGF subunit B) (PDGF-2) (Platelet-derived growth factor B chain) (Platelet-derived growth factor beta polypeptide) (Proto-oncogene c-Sis) (Becaplermin), Resistin (Adipose tissue-specific secretory factor) (ADSF) (C/EBP-epsilon-regulated myeloid-specific secreted cysteine-rich protein) (Cysteine-rich secreted protein A12-alpha-like 2) (Cysteine-rich secreted protein FIZZ3), Stromal cell-derived factor 1 (SDF-1) (hSDF-1) (C—X—C motif chemokine 12) (Intercrine reduced in hepatomas) (IRH) (hIRH) (Pre-B cell growth-stimulating factor) (PBSF) [Cleaved into: SDF-1-beta(3-72); SDF-1-alpha(3-67)]; SDF-1 alpha and SDF-1 beta are encoded by a single gene and arise by alternative splicing. The two proteins are identical except for the four amino acid residues that are present in the carboxy-terminus of SDF-1 beta and absent from SDF-1 alpha., Interleukin-6 receptor subunit beta (IL-6 receptor subunit beta) (IL-6R subunit beta) (IL-6R-beta) (IL-6RB) (CDwl30) (Interleukin-6 signal transducer) (Membrane glycoprotein 130) (gp130) (Oncostatin-M receptor subunit alpha) (CD antigen CD130), Sonic hedgehog protein (SHH) (HHG-1) [Cleaved into: Sonic hedgehog protein N-product; Sonic hedgehog protein C-product], Sialic acid-binding Ig-like lectin 5 (Siglec-5) (CD33 antigen-like 2) (Obesity-binding protein 2) (OB—BP2) (OB-binding protein 2) (CD antigen CD170), Interleukin-1 receptor-like 1 (Protein ST2), Transforming growth factor beta-2 (TGF-beta-2) (BSC-1 cell growth inhibitor) (Cetermin) (Glioblastoma-derived T-cell suppressor factor) (G-TSF) (Polyergin) [Cleaved into: Latency-associated peptide (LAP)], Angiopoietin-1 receptor (EC 2.7.10.1) (Endothelial tyrosine kinase) (Tunica interna endothelial cell kinase) (Tyrosine kinase with Ig and EGF homology domains-2) (Tyrosine-protein kinase receptor TEK) (Tyrosine-protein kinase receptor TIE-2) (hTIE2) (p140 TEK) (CD antigen CD202b), Thrombopoietin (C-mpl ligand) (ML) (Megakaryocyte colony-stimulating factor) (Megakaryocyte growth and development factor) (MGDF) (Myeloproliferative leukemia virus oncogene ligand), Tumor necrosis factor receptor superfamily member 10D (Decoy receptor 2) (DcR2) (TNF-related apoptosis-inducing ligand receptor 4) (TRAIL receptor 4) (TRAIL-R4) (TRAIL receptor with a truncated death domain) (CD antigen CD264), Triggering receptor expressed on myeloid cells 1 (TREM-1) (Triggering receptor expressed on monocytes 1) (CD antigen CD354), Vascular endothelial growth factor C (VEGF-C) (Flt4 ligand) (Flt4-L) (Vascular endothelial growth factor-related protein) (VRP), and Vascular endothelial growth factor receptor 1 (VEGFR-1) (EC 2.7.10.1) (Fms-like tyrosine kinase 1) (FLT-1) (Tyrosine-protein kinase FRT) (Tyrosine-protein kinase receptor FLT) (FLT) (Vascular permeability factor receptor). In some embodiments, it is specifically contemplated that any of these may be included in a yield, but in other embodiments, it is specifically contemplated that any of these may not be included in a yield.

Yet further embodiments of the releasates in accordance with the current include one or more of the following bioactive compounds. C—X—C motif chemokine 13 (Angie) (B cell-attracting chemokine 1) (BCA-1) (B lymphocyte chemoattractant) (CXC chemokine BLC) (Small-inducible cytokine B13), Eotaxin (C—C motif chemokine 11) (Eosinophil chemotactic protein) (Small-inducible cytokine A11), C—C motif chemokine 24 (CK-beta-6) (Eosinophil chemotactic protein 2) (Eotaxin-2) (Myeloid progenitor inhibitory factor 2) (MPIF-2) (Small-inducible cytokine A24), Granulocyte colony-stimulating factor (G-CSF) (Pluripoietin) (Filgrastim) (Lenograstim), Granulocyte-macrophage colony-stimulating factor (GM-CSF) (Colony-stimulating factor) (CSF) (Molgramostin) (Sargramostim), C—C motif chemokine 1 (Small-inducible cytokine A1) (T lymphocyte-secreted protein I-309), Intercellular adhesion molecule 1 (ICAM-1) (Major group rhinovirus receptor) (CD antigen CD54), Interferon gamma (IFN-gamma) (Immune interferon), Interleukin-1 alpha (IL-1 alpha) (Hematopoietin-1), Interleukin-1 beta (IL-1 beta) (Catabolin), Interleukin-1 receptor antagonist protein (IL-1RN) (IL-lra) (IRAP) (ICIL-1RA) (IL1 inhibitor) (Anakinra), Interleukin-2 (IL-2) (T-cell growth factor) (TCGF) (Aldesleukin), Interleukin-4 (IL-4) (B-cell stimulatory factor 1) (BSF-1) (Binetrakin) (Lymphocyte stimulatory factor 1) (Pitrakinra), Interleukin-5 (IL-5) (B-cell differentiation factor I) (Eosinophil differentiation factor) (T-cell replacing factor) (TRF) Interleukin-6 (IL-6) (B-cell stimulatory factor 2) (BSF-2) (CTL differentiation factor) (CDF) (Hybridoma growth factor) (Interferon beta-2) (IFN-beta-2), Interleukin-6 receptor subunit alpha (IL-6 receptor subunit alpha) (IL-6R subunit alpha) (IL-6R-alpha) (IL-6RA) (IL-6R 1) (Membrane glycoprotein 80) (gp80) (CD antigen CD126), Interleukin-7 (IL-7), Interleukin-8 (IL-8) (C—X—C motif chemokine 8) (Chemokine (C—X—C motif) ligand 8) (Emoctakin) (Granulocyte chemotactic protein 1) (GCP-1) (Monocyte-derived neutrophil chemotactic factor) (MDNCF) (Monocyte-derived neutrophil-activating peptide) (MONAP) (Neutrophil-activating protein 1) (NAP-1) (Protein 3-10C) (T-cell chemotactic factor) [Cleaved into: MDNCF-a (GCP/IL-8 protein IV) (IL8/NAP1 form I); Interleukin-8 ((Ala-IL-8)77) (GCP/IL-8 protein II) (IL-8(1-77)) (IL8/NAP1 form II) (MDNCF-b); IL-8(5-77); IL-8(6-77) ((Ser-IL-8)72) (GCP/IL-8 protein I) (IL8/NAP1 form III) (Lymphocyte-derived neutrophil-activating factor) (LYNAP) (MDNCF-c) (Neutrophil-activating factor) (NAF); IL-8(7-77) (GCP/IL-8 protein V) (IL8/NAP1 form IV); IL-8(8-77) (GCP/IL-8 protein VI) (IL8/NAP1 form V); IL-8(9-77) (GCP/IL-8 protein III) (IL8/NAP1 form VI)], Interleukin-10 (IL-10) (Cytokine synthesis inhibitory factor) (CSIF), Interleukin-11 (IL-11) (Adipogenesis inhibitory factor) (AGIF) (Oprelvekin), Interleukin-12 subunit beta (IL-12B) (Cytotoxic lymphocyte maturation factor 40 kDa subunit) (CLMF p40) (IL-12 subunit p40) (NK cell stimulatory factor chain 2) (NKSF2), Interleukin-12 subunit alpha (IL-12A) (Cytotoxic lymphocyte maturation factor 35 kDa subunit) (CLMF p35) (IL-12 subunit p35) (NK cell stimulatory factor chain 1) (NKSF1); Interleukin-12 subunit beta (IL-12B) (Cytotoxic lymphocyte maturation factor 40 kDa subunit) (CLMF p40) (IL-12 subunit p40) (NK cell stimulatory factor chain 2) (NKSF2), Interleukin-13 (IL-13), Interleukin-15 (IL-15), Pro-interleukin-16 [Cleaved into: Interleukin-16 (IL-16) (Lymphocyte chemoattractant factor) (LCF)], Interleukin-17A (IL-17) (IL-17A) (Cytotoxic T-lymphocyte-associated antigen 8) (CTLA-8), C—C motif chemokine 2 (HC11) (Monocyte chemoattractant protein 1) (Monocyte chemotactic and activating factor) (MCAF) (Monocyte chemotactic protein 1) (MCP-1) (Monocyte secretory protein JE) (Small-inducible cytokine A2), Macrophage colony-stimulating factor 1 (CSF-1) (M-CSF) (MCSF) (Lanimostim) [Cleaved into: Processed macrophage colony-stimulating factor 1], C—X—C motif chemokine 9 (Gamma-interferon-induced monokine) (Monokine induced by interferon-gamma) (HuMIG) (MIG) (Small-inducible cytokine B9), C—C motif chemokine 3 (GO/G1 switch regulatory protein 19-1) (Macrophage inflammatory protein 1-alpha) (MIP-1-alpha) (PAT 464.1) (SIS-beta) (Small-inducible cytokine A3) (Tonsillar lymphocyte LD78 alpha protein) [Cleaved into: MIP-1-alpha(4-69) (LD78-alpha(4-69))], C—C motif chemokine 4 (G-26 T-lymphocyte-secreted protein) (HC21) (Lymphocyte activation gene 1 protein) (LAG-1) (MIP-1-beta(1-69)) (Macrophage inflammatory protein 1-beta) (MIP-1-beta) (PAT 744) (Protein H400) (SIS-gamma) (Small-inducible cytokine A4) (T-cell activation protein 2) (ACT-2) [Cleaved into: MIP-1-beta(3-69)], C—C motif chemokine 15 (Chemokine CC-2) (HCC-2) (Leukotactin-1) (LKN-1) (MIP-1 delta) (Macrophage inflammatory protein 5) (MIP-5) (Mrp-2b) (NCC-3) (Small-inducible cytokine A15) [Cleaved into: CCL15(22-92); CCL15(25-92); CCL15(29-92)], Platelet-derived growth factor subunit B (PDGF subunit B) (PDGF-2) (Platelet-derived growth factor B chain) (Platelet-derived growth factor beta polypeptide) (Proto-oncogene c-Sis) (Becaplermin), C—C motif chemokine 5 (EoCP) (Eosinophil chemotactic cytokine) (SIS-delta) (Small-inducible cytokine A5) (T cell-specific protein P228) (TCP228) (T-cell-specific protein RANTES) [Cleaved into: RANTES(3-68); RANTES(4-68)], Metalloproteinase inhibitor 1 (Erythroid-potentiating activity) (EPA) (Fibroblast collagenase inhibitor) (Collagenase inhibitor) (Tissue inhibitor of metalloproteinases 1) (TIMP-1), Metalloproteinase inhibitor 2 (CSC-21K) (Tissue inhibitor of metalloproteinases 2) (TIMP-2), Tumor necrosis factor (Cachectin) (TNF-alpha) (Tumor necrosis factor ligand superfamily member 2) (TNF-a) [Cleaved into: Tumor necrosis factor, membrane form (N-terminal fragment) (NTF); Intracellular domain 1 (ICD1); Intracellular domain 2 (ICD2); C-domain 1; C-domain 2; Tumor necrosis factor, soluble form], Lymphotoxin-alpha (LT-alpha) (TNF-beta) (Tumor necrosis factor ligand superfamily member 1), Tumor necrosis factor receptor superfamily member 1A (Tumor necrosis factor receptor 1) (TNF-R1) (Tumor necrosis factor receptor type I) (TNF-RI) (TNFR-I) (p55) (p60) (CD antigen CD120a) [Cleaved into: Tumor necrosis factor receptor superfamily member 1A, membrane form; Tumor necrosis factor-binding protein 1 (TBPI)], and Tumor necrosis factor receptor superfamily member 1B (Tumor necrosis factor receptor 2) (TNF-R2) (Tumor necrosis factor receptor type II) (TNF-RII) (TNFR-II) (p75) (p80 TNF-alpha receptor) (CD antigen CD120b) (Etanercept) [Cleaved into: Tumor necrosis factor receptor superfamily member 1b, membrane form; Tumor necrosis factor-binding protein 2 (TBP-2) (TBPII)]. In some embodiments, it is specifically contemplated that any of these may be included in a yield, but in other embodiments, it is specifically contemplated that any of these may not be included in a yield.

Additional bioactive substances that may be present in the releasates or compositions of the current disclosure include one or more of the following: Tumor necrosis factor receptor superfamily member 9 (4-1BB ligand receptor) (CDw137) (T-cell antigen 4-1BB homolog) (T-cell antigen ILA) (CD antigen CD137), CD166 antigen (Activated leukocyte cell adhesion molecule) (CD antigen CD166), T-lymphocyte activation antigen CD80 (Activation B7-1 antigen) (BB1) (CTLA-4 counter-receptor B7.1) (B7) (CD antigen CD80), Tumor necrosis factor receptor superfamily member 17 (B-cell maturation protein) (CD antigen CD269), Monocyte differentiation antigen CD14 (Myeloid cell-specific leucine-rich glycoprotein) (CD antigen CD14) [Cleaved into: Monocyte differentiation antigen CD14, urinary form; Monocyte differentiation antigen CD14, membrane-bound form], Tumor necrosis factor receptor superfamily member 8 (CD30 L receptor) (Ki-1 antigen) (Lymphocyte activation antigen CD30) (CD antigen CD30), CD40 ligand (CD40-L) (T-cell antigen Gp39) (TNF-related activation protein) (TRAP) (Tumor necrosis factor ligand superfamily member 5) (CD antigen CD154) [Cleaved into: CD40 ligand, membrane form; CD40 ligand, soluble form], Carcinoembryonic antigen-related cell adhesion molecule 1 (Biliary glycoprotein 1) (BGP-1) (CD antigen CD66a), Tumor necrosis factor receptor superfamily member 21 (Death receptor 6) (CD antigen CD358), Tyrosine-protein kinase receptor TYRO3 (EC 2.7.10.1) (Tyrosine-protein kinase BYK) (Tyrosine-protein kinase DTK) (Tyrosine-protein kinase RSE) (Tyrosine-protein kinase SKY) (Tyrosine-protein kinase TIF), Endoglin (CD antigen CD105), Receptor tyrosine-protein kinase erbB-3 (EC 2.7.10.1) (Proto-oncogene-like protein c-ErbB-3) (Tyrosine kinase-type cell surface receptor HER3), E-selectin (CD62 antigen-like family member E) (Endothelial leukocyte adhesion molecule 1) (ELAM-1) (Leukocyte-endothelial cell adhesion molecule 2) (LECAM2) (CD antigen CD62E), Tumor necrosis factor receptor superfamily member 6 (Apo-1 antigen) (Apoptosis-mediating surface antigen FAS) (FASLG receptor) (CD antigen CD95), Fms-related tyrosine kinase 3 ligand (Flt3 ligand) (Flt3 L) (SL cytokine), Tumor necrosis factor receptor superfamily member 18 (Activation-inducible TNFR family receptor) (Glucocorticoid-induced TNFR-related protein) (CD antigen CD357), Tumor necrosis factor receptor superfamily member 14 (Herpes virus entry mediator A) (Herpesvirus entry mediator A) (HveA) (Tumor necrosis factor receptor-like 2) (TR2) (CD antigen CD270), Intercellular adhesion molecule 3 (ICAM-3) (CDw50) (ICAM-R) (CD antigen CD50), Contactin-2 (Axonal glycoprotein TAG-1) (Axonin-1) (Transient axonal glycoprotein 1) (TAX-1), Interleukin-1 receptor type 1 (IL-1R-1) (IL-1RT-1) (IL-1RT1) (CD121 antigen-like family member A) (Interleukin-1 receptor alpha) (IL-1R-alpha) (Interleukin-1 receptor type I) (p80) (CD antigen CD121a) [Cleaved into: Interleukin-1 receptor type 1, membrane form (mIL-1R1) (mIL-1RI); Interleukin-1 receptor type 1, soluble form (sIL-1R1) (sIL-1RI)], Cytokine receptor common subunit gamma (Interleukin-2 receptor subunit gamma) (IL-2 receptor subunit gamma) (IL-2R subunit gamma) (IL-2RG) (gammaC) (p64) (CD antigen CD132), Interleukin-10 receptor subunit beta (IL-10 receptor subunit beta) (IL-10R subunit beta) (IL-10RB) (Cytokine receptor class-II member 4) (Cytokine receptor family 2 member 4) (CRF2-4) (Interleukin-10 receptor subunit 2) (IL-10R subunit 2) (IL-10R2) (CD antigen CDw210b), Interleukin-17 receptor A (IL-17 receptor A) (IL-17RA) (CDw217) (CD antigen CD217), Interleukin-21 receptor (IL-21 receptor) (IL-21R) (Novel interleukin receptor) (CD antigen CD360), Lysosome membrane protein 2 (85 kDa lysosomal membrane sialoglycoprotein) (LGP85) (CD36 antigen-like 2) (Lysosome membrane protein II) (LIMP II) (Scavenger receptor class B member 2) (CD antigen CD36), Neutrophil gelatinase-associated lipocalin (NGAL) (25 kDa alpha-2-microglobulin-related subunit of MMP-9) (Lipocalin-2) (Oncogene 24p3) (Siderocalin LCN2) (p25), L-selectin (CD62 antigen-like family member L) (Leukocyte adhesion molecule 1) (LAM-1) (Leukocyte surface antigen Leu-8) (Leukocyte-endothelial cell adhesion molecule 1) (LECAM1) (Lymph node homing receptor) (TQ1) (gp90-MEL) (CD antigen CD62 L), Lymphatic vessel endothelial hyaluronic acid receptor 1 (LYVE-1) (Cell surface retention sequence-binding protein 1) (CRSBP-1) (Extracellular link domain-containing protein 1) (Hyaluronic acid receptor), MHC class I polypeptide-related sequence A (MIC-A), MHC class I polypeptide-related sequence B (MIC-B), Pro-neuregulin-1, membrane-bound isoform (Pro-NRG1) [Cleaved into: Neuregulin-1 (Acetylcholine receptor-inducing activity) (ARIA) (Breast cancer cell differentiation factor p45) (Glial growth factor) (Heregulin) (HRG) (Neu differentiation factor) (Sensory and motor neuron-derived factor)], Platelet-derived growth factor receptor beta (PDGF-R-beta) (PDGFR-beta) (EC 2.7.10.1) (Beta platelet-derived growth factor receptor) (Beta-type platelet-derived growth factor receptor) (CD140 antigen-like family member B) (Platelet-derived growth factor receptor 1) (PDGFR-1) (CD antigen CD140b), Platelet endothelial cell adhesion molecule (PECAM-1) (EndoCAM) (GPIIA′) (PECA1) (CD antigen CD31), MAPK/MAK/MRK overlapping kinase (EC 2.7.11.22) (MOK protein kinase) (Renal tumor antigen 1) (RAGE-1), Hepatitis A virus cellular receptor 1 (HAVcr-1) (Kidney injury molecule 1) (KIM-1) (T-cell immunoglobulin and mucin domain-containing protein 1) (TIMD-1) (T-cell immunoglobulin mucin receptor 1) (TIM) (TIM-1) (T-cell membrane protein 1), Tumor necrosis factor receptor superfamily member 10C (Antagonist decoy receptor for TRAIL/Apo-2 L) (Decoy TRAIL receptor without death domain) (Decoy receptor 1) (DcR1) (Lymphocyte inhibitor of TRAIL) (TNF-related apoptosis-inducing ligand receptor 3) (TRAIL receptor 3) (TRAIL-R3) (TRAIL receptor without an intracellular domain) (CD antigen CD263), Elafin (Elastase-specific inhibitor) (ESI) (Peptidase inhibitor 3) (PI-3) (Protease inhibitor WAP3) (Skin-derived antileukoproteinase) (SKALP) (WAP four-disulfide core domain protein 14), Urokinase plasminogen activator surface receptor (U-PAR) (uPAR) (Monocyte activation antigen Mo3) (CD antigen CD87), Vascular cell adhesion protein 1 (V-CAM 1) (VCAM-1) (INCAM-100) (CD antigen CD106), and Tumor necrosis factor receptor superfamily member 27 (X-linked ectodysplasin-A2 receptor) (EDA-A2 receptor). In some embodiments, it is specifically contemplated that any of these may be included in a yield, but in other embodiments, it is specifically contemplated that any of these may not be included in a yield.

In a composition disclosed herein, any of the bioactive compounds or growth factors may have a concentration of about, at least about, or at most about 0.1, 1, 2, 3, 4, 5, 6, 7, 8, 9, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 2000, 3000, 4000, 5000 or more pg/ml (or any range derivable therein) or it may not have a concentration that is detectable in any composition discussed herein, or before or after any step of a method discussed herein. In specific embodiments, any one or more of the bioactive compounds or growth factors is specifically excluded in a composition discussed herein.

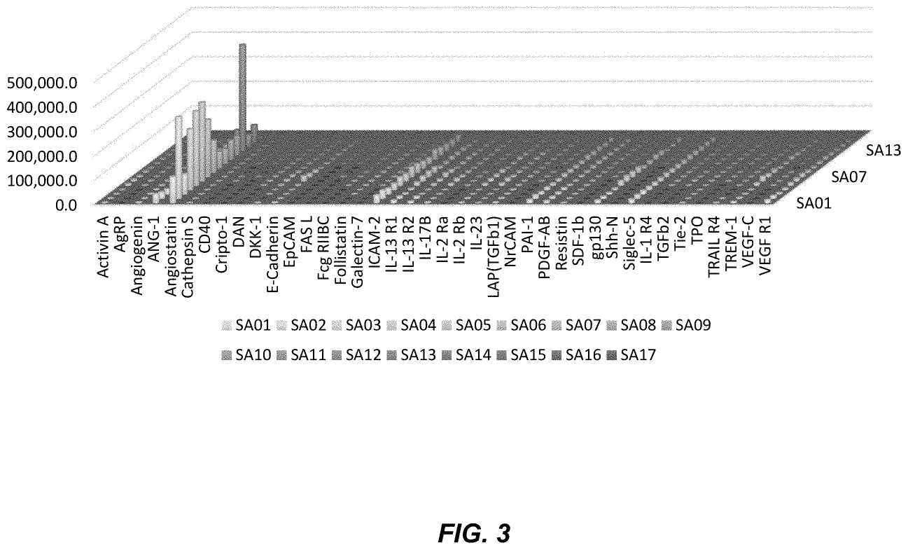

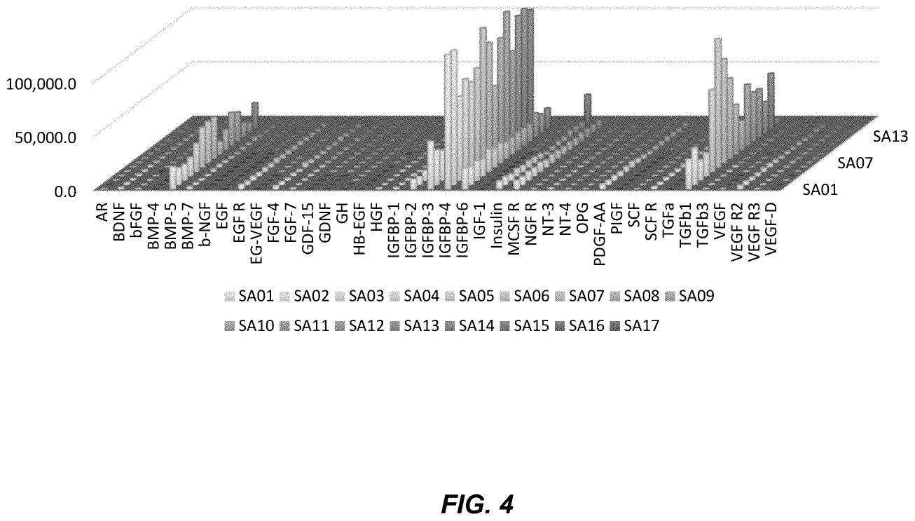

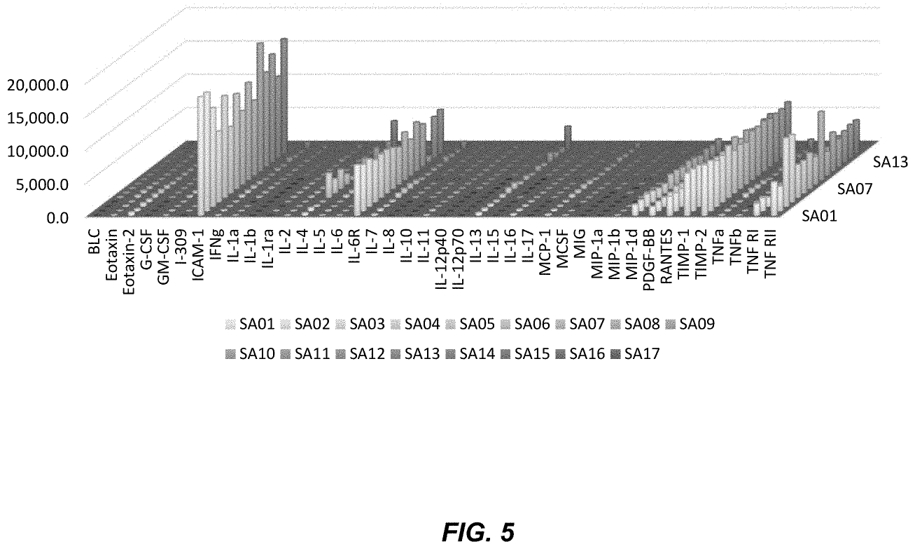

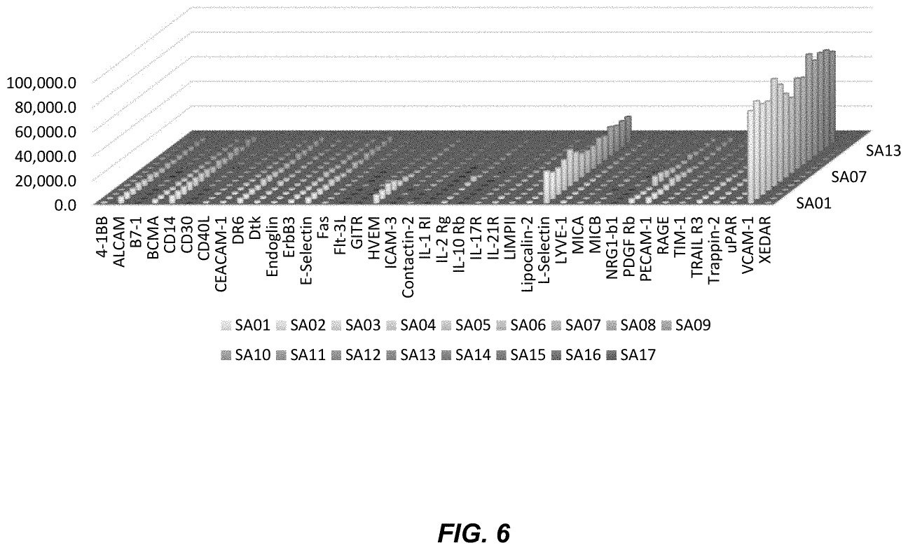

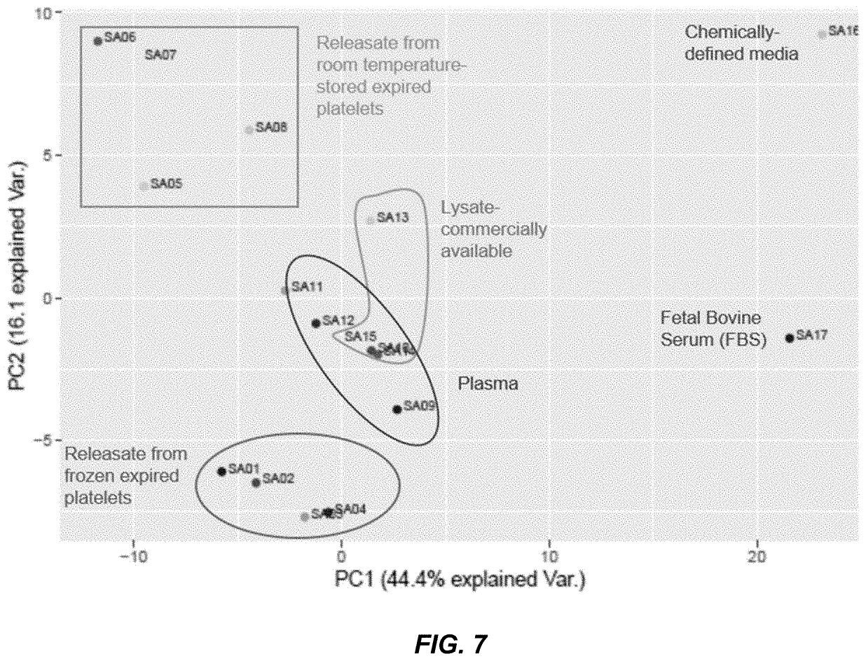

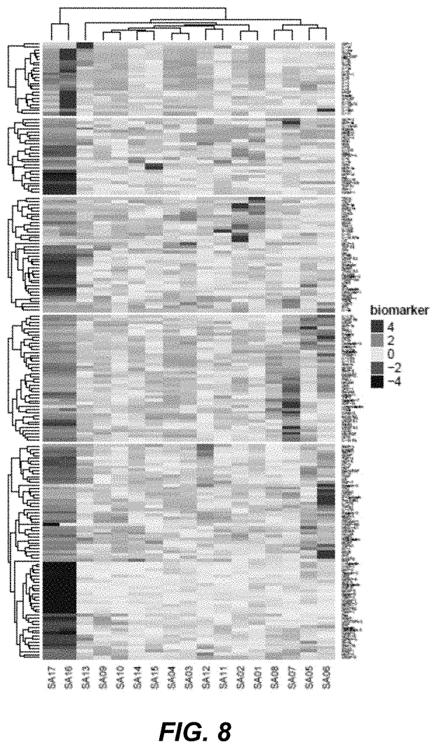

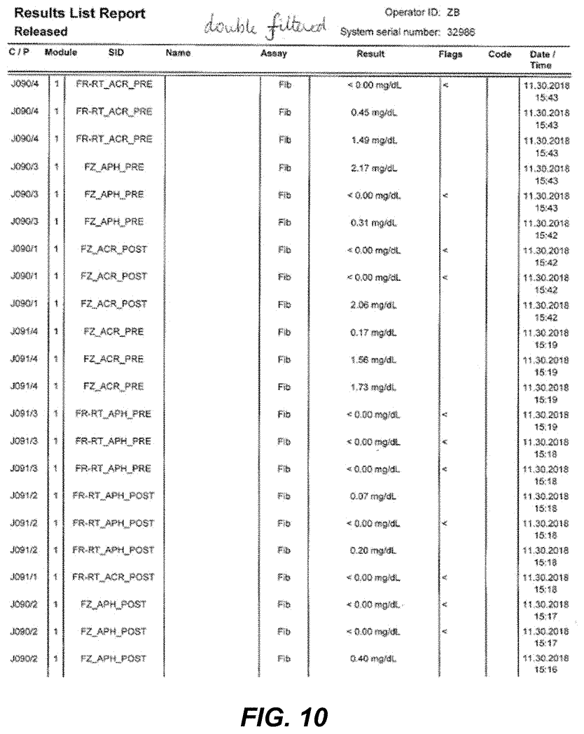

In one example, FIGS. 1A-10 show quantities and identities of various growth factors cytokines, interleukins, interferons in example releasates of the current disclosure compared with plasma, platelet lysates, defined chemical media, or media supplied with FBS. IN a further example, Table 1 shows quantities and identities of various growth factors cytokines, interleukins, interferons in example releasates (releasate F1 and releasate F2) of the present disclosure.

Basic fibroblast growth factor (bFGF), also FGF-β or FGF-basic, is a growth factor and signaling protein encoded by the FGF2 gene. In some embodiments, platelet releasate comprises FGF-basic at a level of at least about 100 pg/ml. In some embodiments, platelet releasate comprises FGF-basic at a level of at least about 200 pg/ml. In some embodiments, platelet releasate comprises FGF-basic at a level of at least about 300 pg/ml. In some embodiments of the methods of the current disclosure, the releasate comprises FGF-basic at a level of at least about 300 pg/ml. In some embodiments, releasate comprises FGF-basic at a level between about 100-800 pg/ml. In some embodiments, releasate comprises FGF-basic at a level between about 300-550 pg/ml. In other embodiments, the releasate comprises FGF-basic at a level between about 350-520 pg/ml. Yet in further embodiments, the releasate comprises FGF-basic at a level between about 400-500 pg/ml. In some embodiments, the releasate comprises FGF-basic at a level of about 450 pg/ml. The releasate may include FGF-basic at a level of about, at least about, or at most about 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 700, or 800 pg/ml or any value or range derivable therein. In some embodiments, it is specifically contemplated that a composition (e.g., a releasate) includes FGF-basic. In other embodiments, it is specifically contemplated that a composition does not include FGF-basic, or does not include a detectable or biologically relevant amount of FGF-basic (e.g., less than 0.1 pg/ml).

In some embodiments of the methods of the current disclosure, the releasate comprises SDF-1α at about 2.0-50 pg/ml. In some embodiments of the methods of the current disclosure, the releasate comprises SDF-1α at about 4.0-30 pg/ml. In some embodiments of the methods of the current disclosure, the releasate comprises SDF-1α at about 5.0-20 pg/ml. In some embodiments, the releasate comprises the releasate comprises SDF-1α at about 7.0-15 pg/ml. In some embodiments, the releasate comprises SDF-1α at about 8.0-14 pg/ml. In other embodiments, releasate comprises the releasate comprises SDF-1α at about 9.0-12.0 pg/ml. In further embodiments, the releasate contains, contains at least, or contains at most 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 30, 31, 32, 33, 34, 35, 36, 37, 38, 39, 40, 41, 42, 43, 44, 45, 46, 47, 48, 49, or 50 pg/ml of SDF-1α or any value or range derivable therein. In some embodiments, it is specifically contemplated that a composition (e.g., a releasate) includes SDF-1α. In other embodiments, it is specifically contemplated that a composition does not include SDF-1α, or does not include a detectable or biologically relevant amount of SDF-1α (e.g., less than 0.1 pg/ml).

In some embodiments of the methods and compositions of the current disclosure, the releasate comprises microvesicles or exosomes from cells. Microvesicles or exosomes may comprise growth factors or other proteins from cells. Microvesicles or exosomes may vary in size and composition. In some embodiments, microvesicles or exosomes comprise one or more of growth factors, cytokines, chemokines, DNA, RNA, microRNA (miRNA), and other nucleic acids. In some embodiments, it is specifically contemplated that releasate comprises microvesicles. In other embodiments, it is specifically contemplated that a composition does not include microvesicles, or does not include a detectable or biologically relevant amount of microvesicles. In some embodiments, it is specifically contemplated that releasate comprises exosomes. In other embodiments, it is specifically contemplated that a composition does not include exosomes, or does not include a detectable or biologically relevant amount of exosomes.

It is contemplated herein that any data presented may include some standard deviation of around 2-10%. This standard deviation is contemplated to apply across all data from the high-through put growth factors, to cytokines and chemokine analyses. In some embodiments, a value of one or more standard deviations is applied in evaluating an acceptable or expected amount of cytokine or biomarker in a composition. In some embodiments, a value of 1, 2, 3, 4, 5, 6, or more standard deviations, is applied.

In general the present disclosure provides methods for large-scale manufacturing of platelet releasate (hPR) from expired units of blood platelets with cGMP. In some embodiments, the method produces hPR with negligible amounts of fibrinogen. In some embodiments hPR comprises less than about 0.05 mg/dL fibrinogen. In some embodiments, hPR comprises any amount between about 0.00-0.05 mg/dL fibrinogen. Fibrinogen is removed by its conversion to a fibrin clot that also traps other cellular debris resulting in a clear supernatant that is hPR.

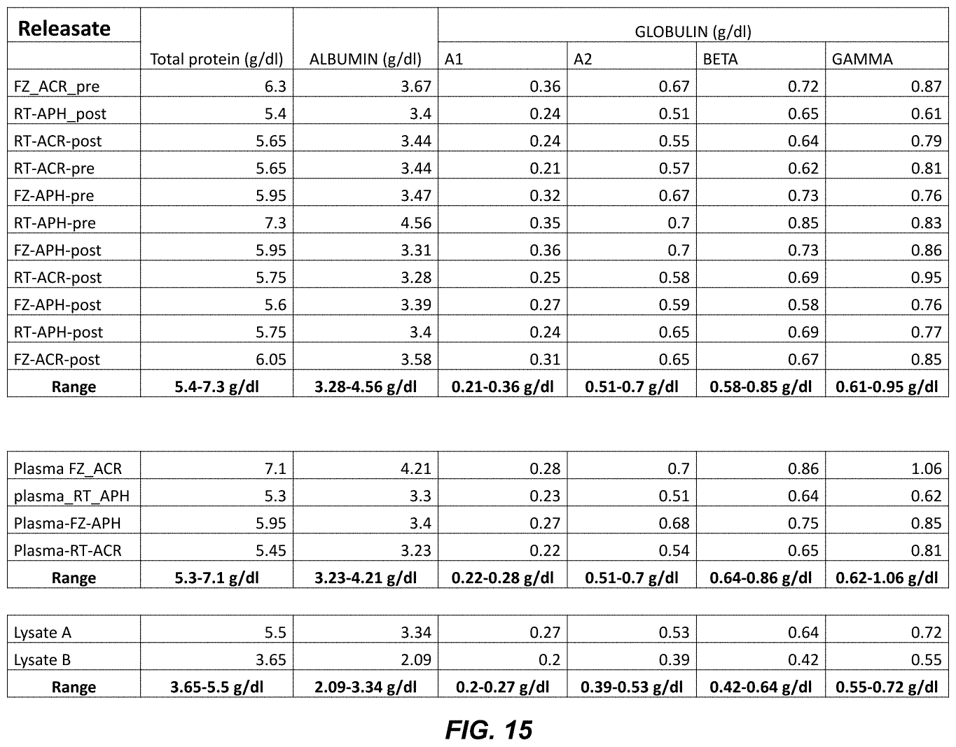

The hPR, in accordance with the current disclosure, may retain other plasma derived components such as globulins and albumins as shown in FIG. 15.

In certain embodiments, the releasate compositions of the current disclosure are added to other ingredients, nutrients or media to form a cell culture or cell storage medium. A releasate may be added to an existing cell culture medium, for example Minimum Essential Medium (MEM) or Dulbecco's Modified Eagle Medium (DMEM), and used for cell culture. A cell culture medium according to the present disclosure is formulated to provide nutrients (e.g. growth factors, etc.) necessary for the growth or maintenance of cells including for example stem and/or progenitor cells, such as mesenchymal stem cells. Such a cell culture medium, in some embodiments, is free from added heparin and any clotted material.

Closed Systems

The methods of preparing hPR, according to the current disclosure, may be performed in a closed system ensuring a contaminant-free final product. A closed system may comprise, for example, prefiltration modules of whole blood or platelet rich plasma, platelet retention modules, mycoplasma and virus-retention modules, etc. The modules may be connected via modular connections for the addition and removal of byproducts. In some embodiments, bags such as those used in blood banking or bioreactors are used. Such bags may be connected to other bags and filtration systems by tubing. The connections are made, for example, using sterile-welders and tube sealers. A closed system may alleviate the need for a clean room. If cells collected for transfusion are never exposed to the surrounding atmosphere, the contents of the bags (i.e. cells) remain sterile. In extension, all unit operations and down-stream processes conducted in this fashion (i.e. by sterile-welding one bag to another or filters) would maintain the sterility of the contents.

Although some embodiments comprise the use of a closed system, in some embodiments, the releasate is prepared in an open system, that is using flasks, tubes, cell culture reservoirs, bioreactors, etc., which may be inside a biosafety cabinet, an isolator, or a clean room. Therapeutic Uses/Formulations/Combinations

In some embodiments, the hPR compositions of the current disclosure may be used as a therapeutic substance. Some aspects of the current disclosure relate to formulations comprising the releasate described herein. The formulations may be used for therapeutic purposes such as for the treatment of damaged, wounded or diseased tissue including but not limited to bone, muscle, skin, nerves, tendons, connective, ocular, periodontal or cardiovascular tissues. hPR can be used as an improved alternative to lysate in clinical applications.

The compositions of the current disclosure may be formulated in any suitable manner for medical treatment including but not limited to liquids, gels, powder, ointments, aerosols, spray, etc. The compositions described herein may be delivered to tissues by any suitable measure including but not limited to surgical implantations, injections, topical application, wound dressing etc.

In some embodiments, hPR is combined with any known osteobiologics and coagulation agents. The former will expedite healing mechanism due to the presence of growth factors, cytokines and chemokines, while the latter may have an application in trauma, field care etc. In some embodiments, hPR (as is or in combination with other agents) can be used to coat tissue culture plastic, microcarriers etc to favor cell adhesion, differentiation and expansion)]. In some embodiments, hPR of the current disclosure are combined with biomaterials, mammalian tissue, engineered or manipulated cells and tissues

In some embodiments, the current disclosure provides a method of treating a subject comprising administering to the subject a composition comprising stem cells, wherein the stem cells have been cultured with a releasate from human blood-derived platelets, wherein the releasate comprises fibrinogen at a level of less than about 0.05 mg/dL, less than about 0.05 mg/dL. In some embodiments, the subject suffers from bone disease, bone defect, bone injury, osteoporosis, osteoarthrosis or spinal cord injury. In some embodiments the subject suffers from cartilage disease or cartilage defect or cartilage injury. In some embodiments, the subject suffers from a bone, tendon, cartilage, or muscle injury. In some embodiments, the subject suffers from periodontal disease. In some embodiments, the subject suffers from an autoimmune disease. In some embodiments, the subject suffers from myocardial infarction. In certain embodiments the releasate is allogeneic with respect to the target patient, in other embodiments the releasate is autogenic or xenogenic to with respect to the target patient.

In certain aspects, the actual dosage amount of a composition administered to a subject or patient can be determined by physical and physiological factors such as size of the damaged or wounded tissue, severity of the condition, the type of condition being treated, previous or concurrent therapeutic interventions, idiopathy of the patient and route of administration.

Platelet-Rich Fibrin

In addition to the releasate of the current disclosure, which may be derived from fresh or expired platelet rich plasma or platelet concentrate, another valuable product, the fibrin clot, may be used for therapeutic purposes. The solid or gel-like clot material is rich in growth factors, and contains sufficient amounts of and clotting factors to serve as a clottable structure, for medical applications.

In some aspects, the disclosure relates to methods for preparing platelet-rich fibrin from fibrinogen a mammalian blood-derived platelet concentrate, the method comprising the steps of: i) obtaining platelets of the human blood thereby obtaining platelet-rich-plasma (PRP); ii) adding CaCl₂) to the PRP to a final concentration of greater than 25 mM; and iii) agitating the CaCl₂/PRP mixture for less than 6 hours thereby forming a fibrin clot and a supernatant; iv) adding an anti-fibrinolytic agent to prevent fibrinolysis; and v) removing the supernatant to obtain the platelet-rich fibrin. In some embodiments, the method further comprises concentrating by removing excess plasma. In some embodiments the mammalian blood is human, equine, canine, feline, porcine, bovine, chicken, feline, porcine, rabbit, dolphin, ovine, murine, rat, simian blood, from a sport animal, from a farm animal, or from a pet. Any step(s) discussed herein in the context of hPR can be implemented in methods for producing platelet-rich fibrin. In certain embodiments, a releasate-producing method further includes isolating or separating a resultant clot. In particular embodiments, methods comprise one or both of the following steps: adding an anti-fibrinolytic agent to the separated clot to prevent fibrinolysis and removing the supernatant to obtain the platelet-rich fibrin. Anti-fibrinolytic agents include, but are not limited to, aprotinin, tranexamic acid, aminomethylbenzoic acid, or aminocaproic acid. Serpins are also anti-fibrinolytic and examples include aprotinin, alpha1 antitrypsin, C1-inhibitor and camostat. In some embodiments, the concentration of the antifibrinolytic agent added or its final concentration with the separated clot is about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 μM or mM (or any range derivable therein). In some embodiments, the concentration of aprotinin ranges between about 1.0 micromolar to about 10 millimolar; tranexamic acid ranges from about 1 micromolar to about 100 millimolar; aminocaproic acid ranges from about 0.01 millimolar to about 50 millimolar.

Therapeutic or medical applications for a fibrin clot are known of a person of skill in the art. They extend to many different fields including orthopedics, sports medicine, regenerative dentistry, cosmetic, plastic and maxillofacial surgery. Such applications include but are not limited to uses as or with biological adhesives, biologic hydrogels, topical formulations for skin care and/or hemostatic agents that prevent blood loss at sites of vascular injury. The growth factors and cytokines in the fibrin clot can play a role, for example, in wound healing, new or rapid vascularization of healing tissue, bone regeneration or soft-tissue maturation.

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications as cited throughout this application) are hereby expressly incorporated by reference. When definitions of terms in documents that are incorporated by reference herein conflict with those used herein, the definitions used herein govern.

### III Examples

### Example 1—A Method for Preparing Human Platelet Releasate

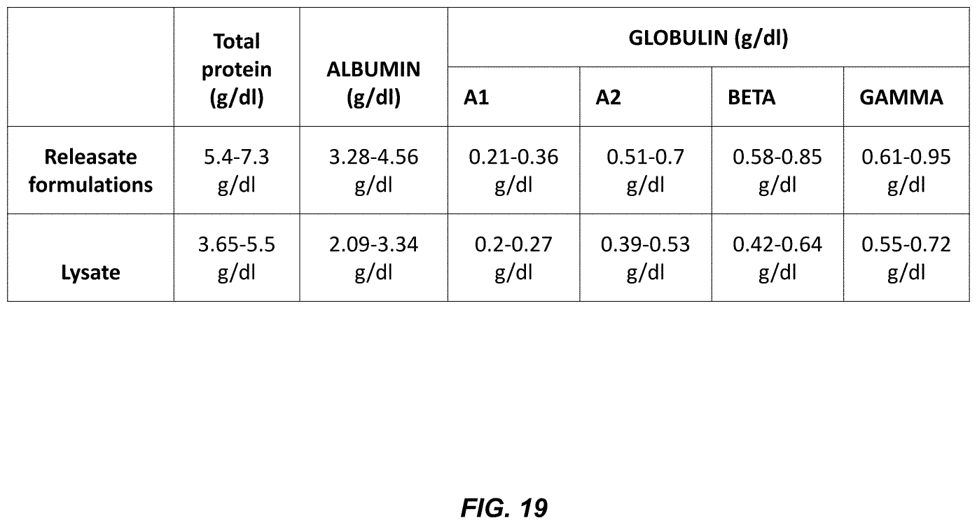

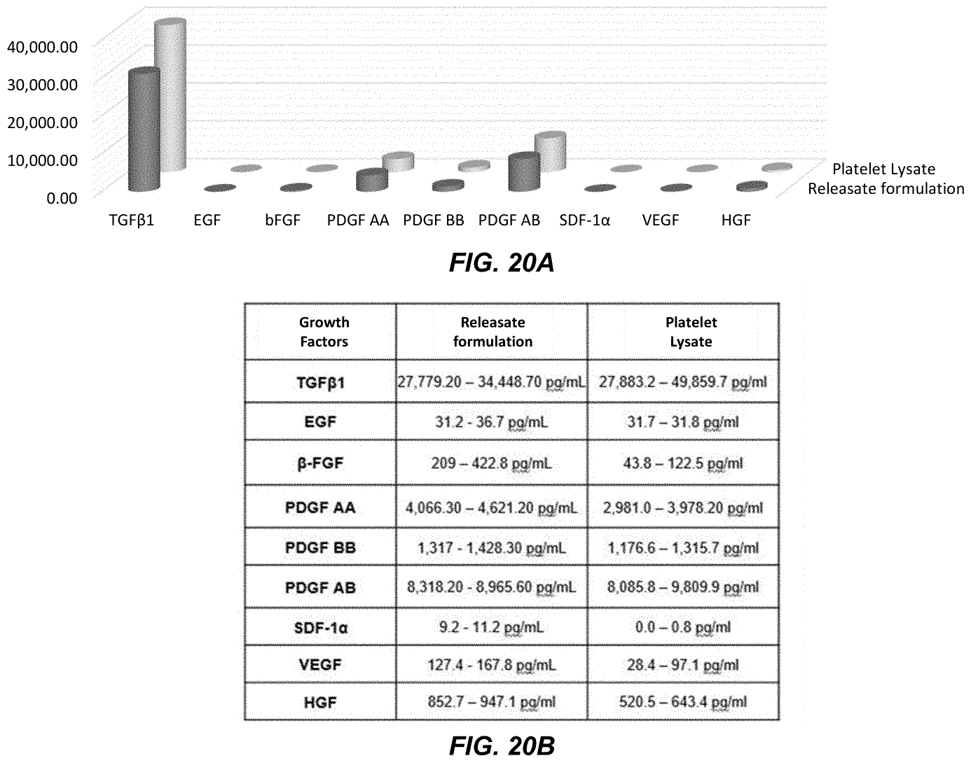

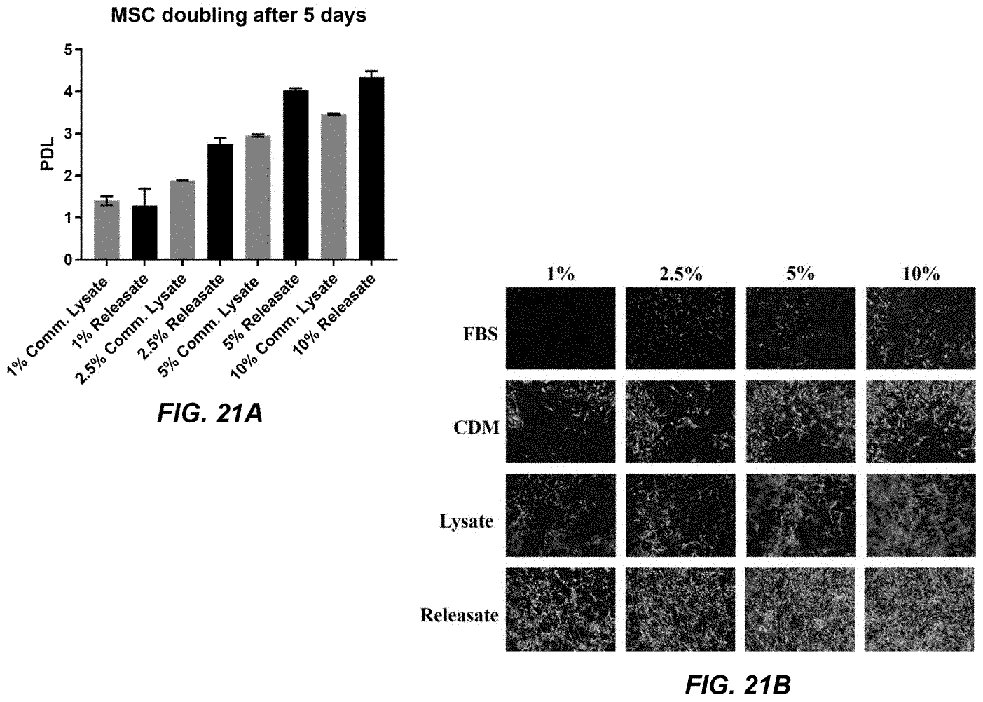

hPR is manufactured in a closed system using expired units of adult blood-derived platelets. Briefly expired units of platelets are fractionated to isolate platelet rich plasma (PRP). After leukoreduction, the platelets were pelleted by centrifugation at 4000 g for 10 min. Based on results of ABO typing and infectious disease testing, the platelets were pooled into batches. The pools of PRP were agitated to release growth factors. The step was followed by another round of centrifugation at 4000 g for 15 minutes to separate cell debris and clotting factors. The supernatant, which is hPR, was collected in aliquots and stored at −80° C. The batches of hPR were characterized by measuring i) total protein, ii) growth factor concentration and iii) growth kinetics of MSC, as shown in FIG. 19, FIGS. 20A and 20B, and FIGS. 21A and 21B.

The following is an example of a protocol for preparing human platelet releasate in accordance with the current disclosure.

1. Units of (Platelet Rich Plasma) PRP were weighed.
2. The following parameters or test results were recorded for each unit used: ABO-type, blood cell count and infectious disease marker (IDM.
3. Only O-type (Rh+ and Rh−) PRP were used as an example but any ABO blood type can be used.
4. Expired units were pooled to a final volume of 250 ml per bag.
5. Each PRP unit was connected to 2 additional empty transfer bags
6. The PRP units were balanced in a centrifuge set for 4000 g at 4° C.
7. After centrifugation for 15 min, the units were gently transferred to a G4/G5 expressor system.
8. the supernatant was expressed i.e. Plasma into an empty bag.
9. The plasma bags were stored at −80° C. until further use.
10. The second bag was used to store concentrated PRP (50 ml or higher) for further processing.
11. The bags were frozen to −55° C. using the rapid cooling freezer.
12. The bags were incubated overnight in −80° C. freezer.
13. Units were incubated until this step to attain a critical manufacturing volume.
14. The bags were thawed for 30 min using a water bath set at 37° C.
15. The platelets were activated by injecting calcium chloride at a final concentration of 40 mM using sterile-welded connections.
16. The contents of the bag were agitated by placing the units on an orbital shaker set-up at 38-40° C., 250 rpm for 90 min.
17. Following the agitation step, the concentrated PRP units were balanced in a centrifuge set for 4000 g at 4° C.
18. The units were centrifuged for 15 min.
19. Using G4/G5, the supernatant in every bag (50 ml) was transferred into a 1-liter bag
20. Units of PRP were pooled to a maximum volume of 500 ml into the 1-liter bag.
21. The units were then incubated in 1 L bag containing 500 ml of unfiltered human platelet releasate (UN-hPR) at 4° C. until further use.
22. From this step on, all operations were conducted inside a bio-safety cabinet (BSC)
23. The bags were transferred to a bio-safety cabinet (BSC), which has a vacuum-driven filtration system (Stericup) for 3-step filtration.
24. 500 ml of UN-hPR were transferred into 0.45 micron Stericup.
25. After filtration, bottle of the Stericup system was collected and its contents were transferred to 0.22 micron Stericup for the second round of filtration.
26. After filtration, the contents transfer bottle of the 0.22 micron Stericup system was collected and transferred to a 0.10 micron Stericup for the third round of filtration.
27. After filtration, the transfer bottle of the 0.10 micron Stericup system was collected and aliquots of 25 ml of filtered human platelet releasate (Fil-hPR) were transferred into 30 ml storage bottles.
28. The storage bottles (20 bottles for every 500 ml) were transferred to a freezer set at −20° C. or below −80° C.

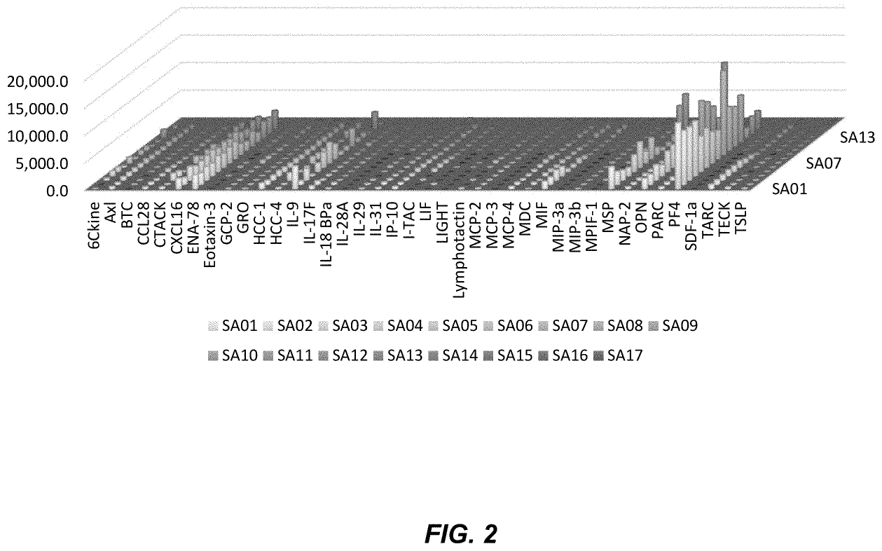

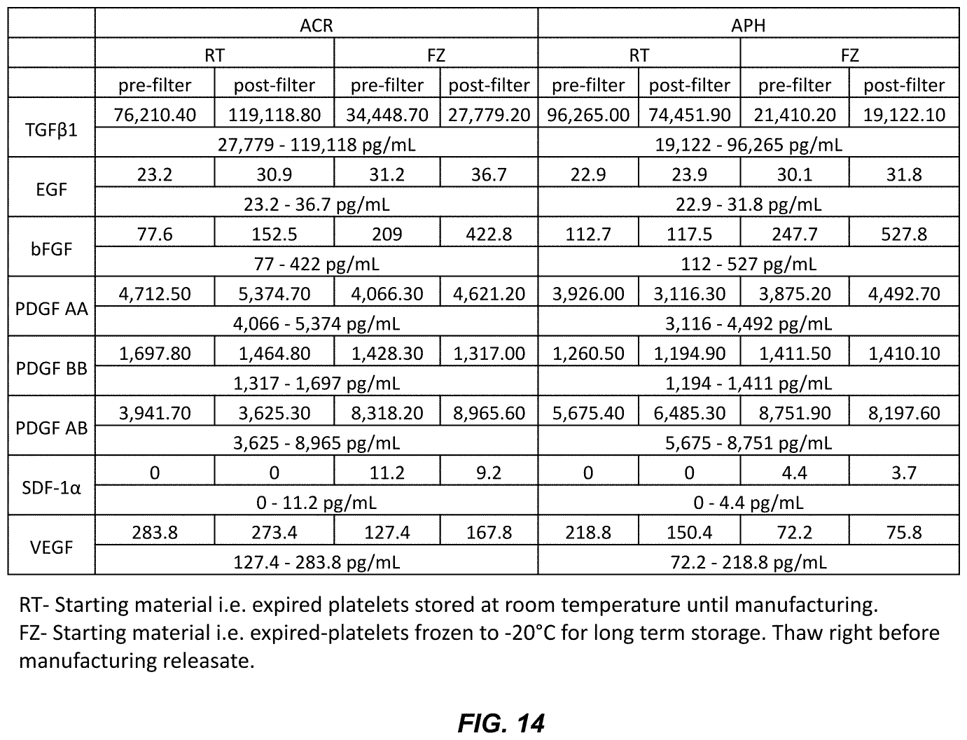

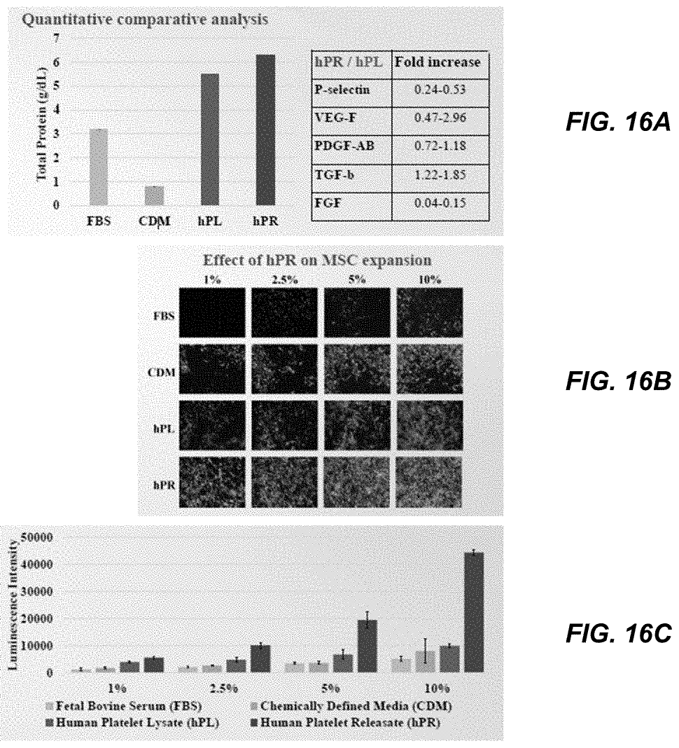

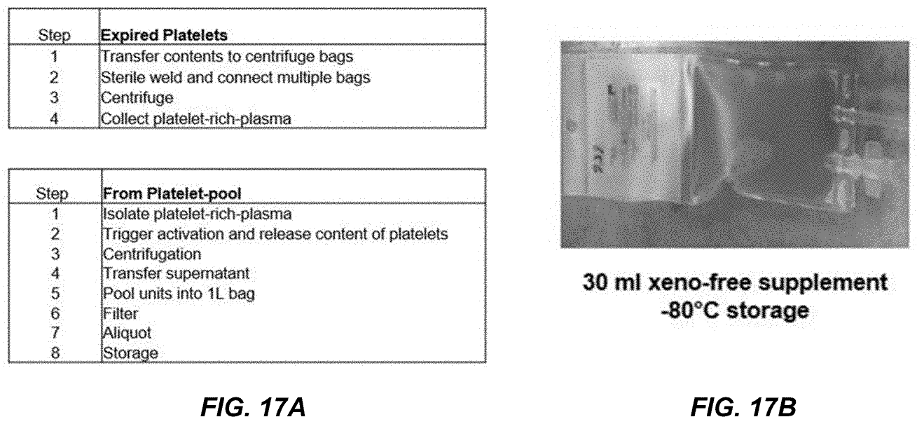

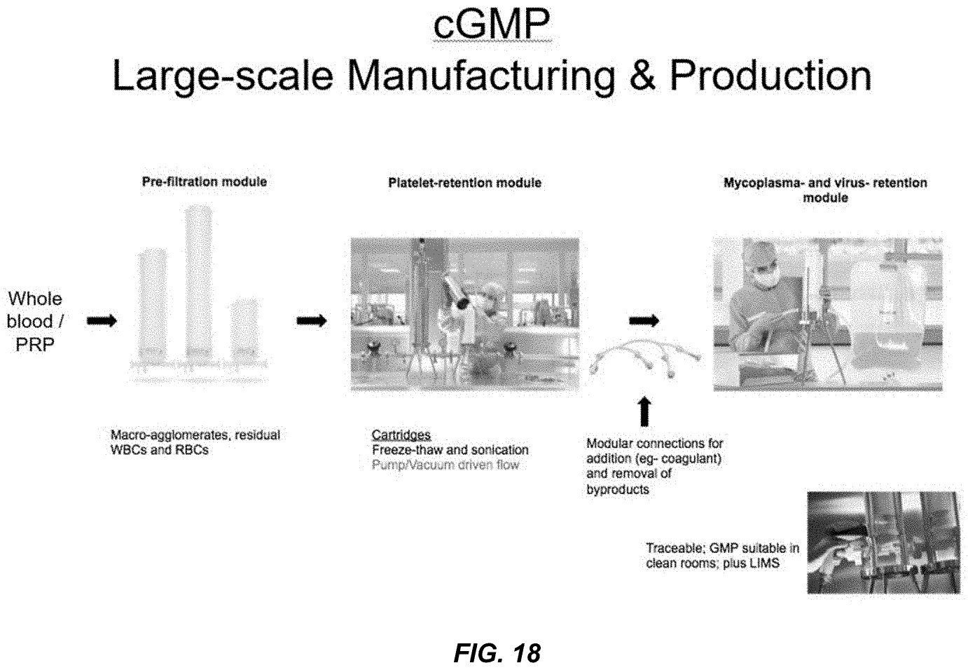

FIGS. 2-6 and 10-20 depict the quantitative comparative analysis of various growth factors, cytokines, interferons, total proteins, globulins, albumins and other components in hPR batches obtained using the protocol described supra. The data shows that unexpectedly the amount of growth factors and cytokines were higher in hPR produced by the current method as compared to controls including commercially available human platelet lysates (hPL), chemically defined media (CDM) and fetal bovine serum (FBS). The results also indicate that the proliferation rate of MSCs is higher in basal medium supplemented with hPR compared to FBS, CDM and hPL.

Variations of the protocol that are in accordance with the current disclosure include filtering the releasate using 0.45-0.65 micron filters. Recent data suggest that it is possible to filter releasate as a part of the closed system.

### Example 2—a Scaled Up Method for Preparing Human Platelet Releasate

The inventors developed a process to manufacture consistent lots of hPR that does not require the addition of anticoagulants to the growth medium. The yield of human platelet releasate using the protocol outlined in Example 1 or modifications thereof, when scaled up, can yield up to 10 L of hPR in less than 4 hours. The impact of hPR in large-scale cell expansion systems was also tested. In large-scale manufacturing of hPR, numbers as high as 24 L every 4 h hours can be obtained.

### Example 3—the Effect of CaCl₂) Concentration on Fibrin Clot Formation

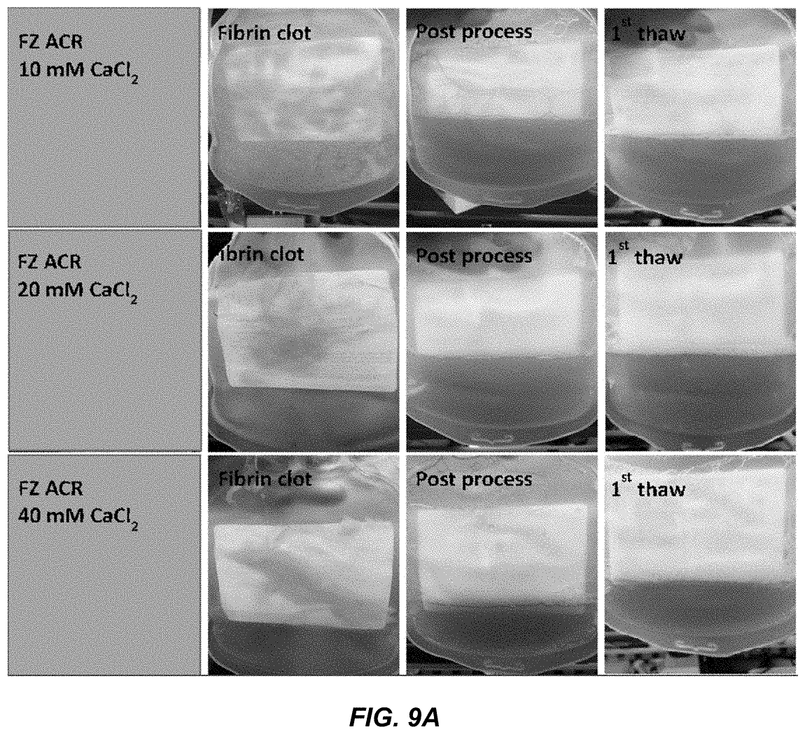

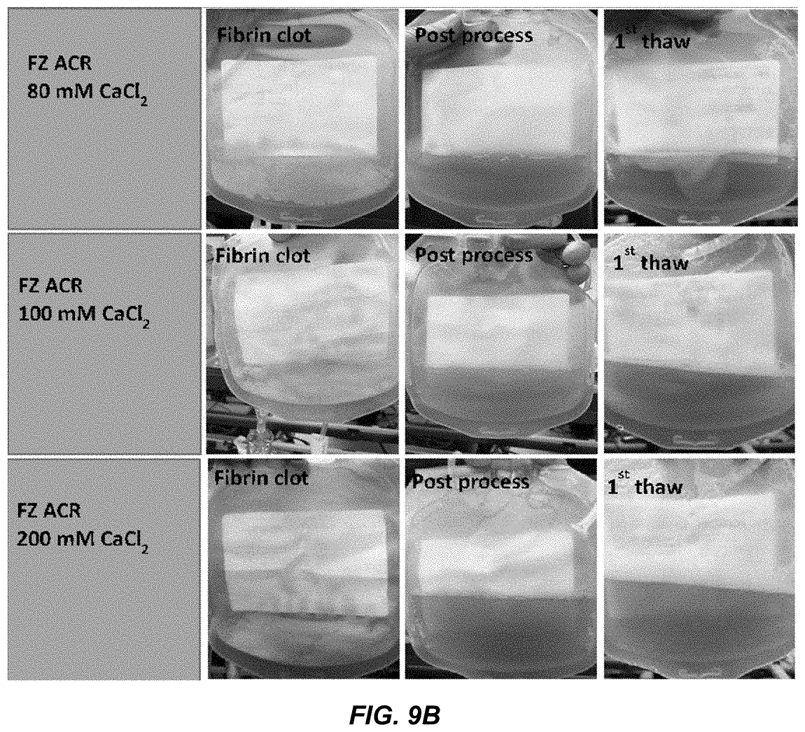

The inventor tested various concentrations of CaCl₂) on clot formation when using the protocol of Example 1. Concentrations that were tested include 10, 20, 40, 80, 100 and 200 mM. Unexpectedly, low concentrations of less than 10 mM (close to prior art recommendations) did not yield a well-defined fibrin clot encapsulating cells. Similarly, higher concentrations of CaCl₂, i.e. greater than 80 mM, did not result in a well-defined fibrin clot and a clear supernatant (releasate). Optimal CaCl₂) concentrations were in the range of 25-80 mM. At such concentrations, the fibrin clot is a well-defined gel that comprises all cellular debris leaving the supernatant as a clear liquid. Data is shown in FIGS. 9A-9B.

### Example 4—the Effect of CaCl₂ Concentration in hPR on MSC Doubling

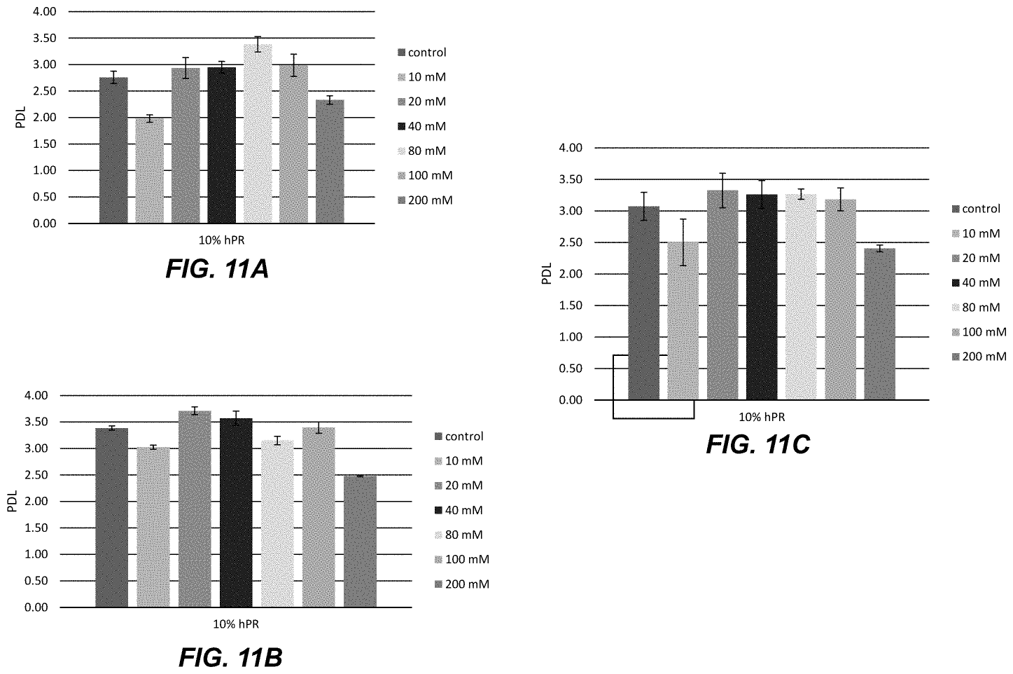

The inventor studied the effect of 10, 20, 40, 80, 100 and 200 mM CaCl₂) in hPR on the population doubling level of bone marrow-derived mesenchymal stem cells. For this experiment, expired Acrodose or Apheresis platelets that were stored at room temperature were used. The control was cells cultured in media with 10% of a commercially available xeno-free alternative to FBS. Cell titer Glo luminescence assay was performed on day 5 and day 7 cell cultures. The data is shown in FIG. 11. Concentrations that yielded the highest PDL levels were 20 and 40 mM CaCl₂)

### Example 5—the Effect of Agitation Duration on Fibrin Clot Formation

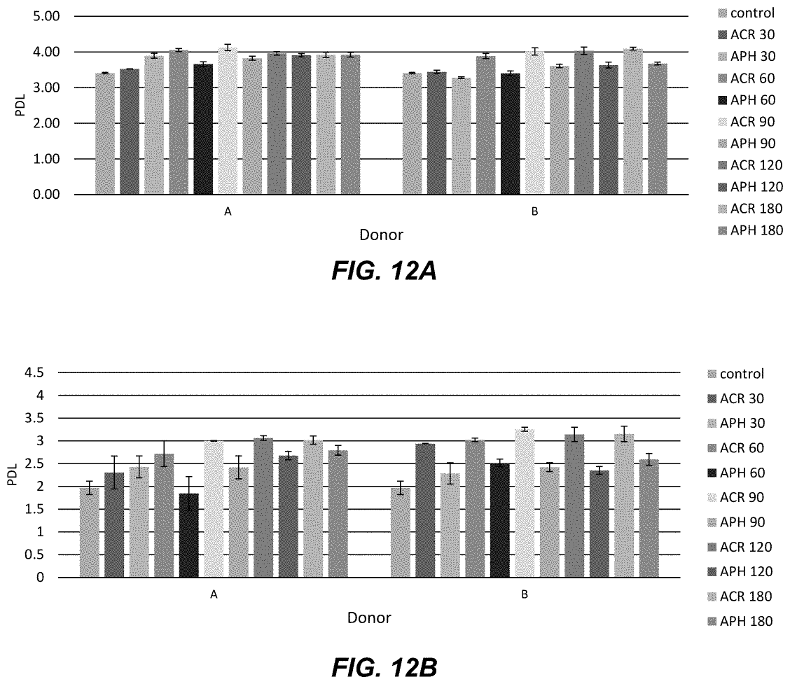

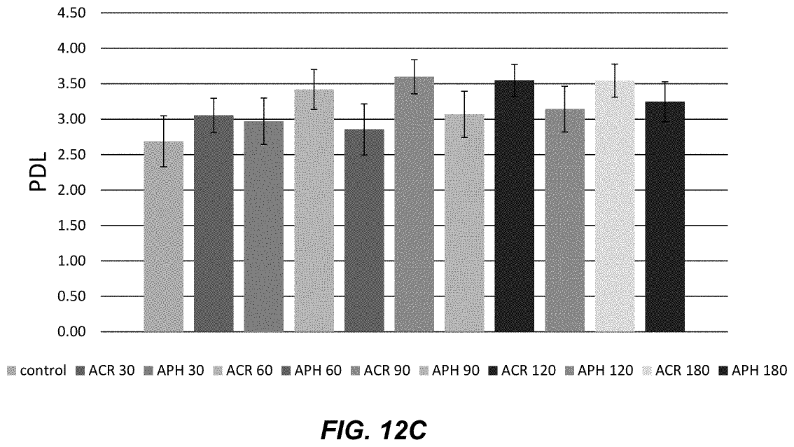

The inventor studied the effect of platelet agitation duration on the population doubling level of bone marrow-derived mesenchymal stem cells. For this experiment, expired Acrodose or Apheresis platelets that were stored at room temperature were used. The control was cells cultured in media with 10% of a commercially available xeno-free alternative to FBS. Cell titer Glo luminescence assay was performed at PDL 3 and PDL 7 time points. The data is shown in FIG. 12. Agitating for 60-90 minutes was shown to be sufficient and durations of up to 180 minutes gave similar results.

### Example 6—the Effect of hPR Centrifugation Duration on MSC Doubling

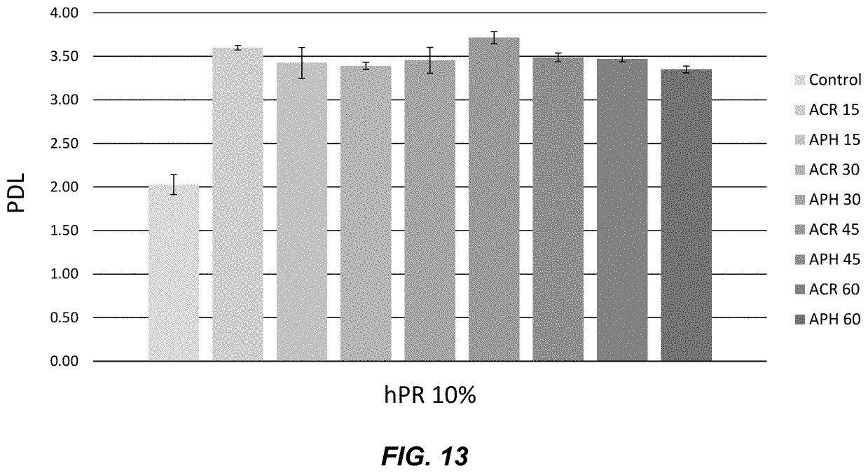

The inventor also tested the effect of hPR centrifugation duration on the population doubling level of bone marrow-derived mesenchymal stem cells. For this experiment, expired Acrodose or Apheresis platelets that were stored at room temperature were used. The control was cells cultured in media with 10% commercial, FBS alternative. Cell titer Glo luminescence assay was performed at PDL 3 time point. The data is shown in FIG. 13. The results show that centrifugation for 15 minutes was an ample enough duration for generating hPR that gave adequate population doubling levels.

### Example 7—the Effect of Using Releasate Supplement Duration on MSC Doubling

Human bone marrow-derived MSCs were cultured in α-MEM supplemented with commercially available human platelet lysates and various human platelet releasate preparations at concentrations of 0.5%, 1%, and 2.5%., 5% and 10%. Cell culture supplements were normally used in the range of 1%-20% suspended in cell culture media. Cells were seeded in 24-well tissue culture plates at a seeding density of 5,000 cells/well. Cell doubling was monitored over 5 days and assessed using the CellTiter Glo luminescence assay. The results, shown in FIGS. 21A and 21B, indicate that the proliferation rate of MSCs is superior in media supplemented with hPR compared to FBS, CDM and hPL. In some instances, the amount of commercial lysates used to reach the same PDL level obtained by hPR was double or more.

### Example 8—Manufacturing or Preparing Human Platelet-Rich-Fibrin (hPRF)

The following is an example of a protocol for preparing human platelet-rich-fibrin in accordance with the current disclosure.

1. Units of (Platelet Rich Plasma) PRP were weighed.
2. The following parameters or test results were recorded for each unit used: ABO-type, blood cell count and infectious disease marker (IDM.
3. Only O-type (Rh+ and Rh−) PRP were used as an example but any other ABO type can be used.
4. Expired units were pooled to a final volume of 250 ml per bag.
5. Each PRP unit was connected to 2 additional empty transfer bags
6. The PRP units were balanced in a centrifuge set for 4000 g at 4° C.
7. After centrifugation for 15 min, the units were gently transferred to a G4/G5 expressor system.
8. The supernatant is expressed i.e. Plasma into an empty bag.
9. The plasma bags were stored at −80° C. until further use.
10. The second bag was used to store concentrated PRP (50 ml or higher) for further processing.
11. The bags were frozen to −55° C. using the rapid cooling freezer.
12. The bags were incubated overnight in −80° C. freezer.
13. Units were incubated until this step to attain a critical manufacturing volume.
14. The bags were thawed for 30 min using a water bath set at 37° C.
15. The platelets were activated by injecting calcium chloride at a final concentration of 40 mM using sterile-welded connections.
16. The contents of the bag were agitated by strapping the units to an orbital shaker set-up at 38-40° C., 250 rpm for 90 min.
17. Following the agitation step, the concentrated PRP units were balanced in a centrifuge set for 4000 g at 4° C.
18. The units were centrifuged for 15 min.
19. Using G4/G5, the supernatant is discarded.
20. The sediment is transferred to a homogenizer.
21. A suitable concentration of anti-fibrinolytic agent is added (for example, aprotinin or tranexamic acid or aminocaproic acid, etc.) to prevent fibrinolysis
22. The platelet-rich fibrin is transferred to sterile containers (such as in a 5 cc volume) and stored at 4° C.

### Example 9—Preparation of Platelet Releasate Formulations

### Preparation of an Example Releasate (Releasate FI)

The following is an example of a protocol for preparing human platelet releasate (releasate F1) in accordance with the current disclosure.
1. Units of (Platelet Rich Plasma) PRP were weighed.
2. The following parameters or test results were recorded for each unit: ABO-type, blood cell count and infectious disease marker (IDM).
3. Only O-type (Rh+ and Rh−) PRP were used in this example but any ABO blood type can be used.
4. PRP units were adjusted to a final volume of 250 ml per bag.
5. Each weight-adjusted PRP unit of 250 mL was connected to an empty transfer bag of 300 ml capacity using a sterile tubing welder.
6. The PRP units were balanced in a centrifuge set for 4000 g at 4° C.
7. After centrifugation for 15 min, the units were gently transferred to an extractor system e.g. Compomat G5 by Fresenius Kabi.
8. The supernatant i.e. plasma was transferred into an empty transfer bag; leaving 50 mL of plasma remaining with the platelets.
9. The transfer bag containing 200 mL transferred-plasma were stored at −80° C. for other use or discard.
10. The bag containing platelets with 50 ml of plasma i.e. concentrated PRP was used for further processing.
11. The bags containing concentrated PRP were first frozen to −55° C. to −80° C. using the rapid cooling freezer.
12. The bags containing concentrated PRP were then transferred to −80° C. freezer for storage.
   NOTE: Concentrated PRP units were collected, processed and stored at −80° C. until a critical manufacturing volume was attained. This storage facilitates accumulation of concentrated PRP units for large-scale manufacturing.
13. The bags containing concentrated PRP were removed from the −80° C. freezer and thawed for 30 min using a water bath set at 37° C.
   NOTE: Steps 11, 12 and 13 may be skipped upon abundant availability of PRP units at step 1 for large-scale manufacturing
14. The platelets were activated by the injection of a sterile calcium chloride solution at a final concentration of 40 mM using sterile-welded connections.
15. The contents of the bag were agitated by placing the units on an orbital shaker set-up at 38° C.-40° C., and 250 rpm for 90 min.
16. Following the agitation step, the calcium chloride-treated concentrated PRP units were balanced in a centrifuge set for 4000 g at 4° C.
17. The units were centrifuged for 15 min.
18. A new collection bag system was assembled by sterile welding/connecting a 1 L transfer bag to a 170-260 micron filter system. For e.g. Fenwal γ-type blood component recipient set containing 170-260 micron filter. This transfer bag assembly will be used in the next step to collect and pool the human platelet releasate.
19. Using the extractor system, the supernatant (referred to as human platelet releasate) in every bag was transferred into the collection bag system.
20. At 50 mL of human platelet releasate per unit, 10 units were pooled to a maximum volume of 500 ml into the 1-liter transfer bag of the collection bag system.
21. The pooled unit were then stored in 1 L bag containing 500 ml human platelet releasate at −80° C. overnight.
22. After overnight treatment at −80° C., the human platelet releasate was thawed for 30 min in a 37° C. water bath
23. The thawed human platelet releasate was connected to a leukoreduction system using a sterile tube welder. The filtered human platelet releasate was collected in the attached 1 L transfer bag
24. Further, aseptic filtration of the human platelet releasate was performed using a 0.65 um in-line filter (e.g. Macopharma in-line filter) that was attached to the 1 L transfer bag containing the product
25. After filtration, aliquots of 25 ml of filtered human platelet releasate were transferred into 30 ml storage bottles.
26. The storage bottles (20 bottles for every 500 ml) were transferred to a freezer set at or below −80° C.

### Preparation of an Example Releasate (Releasate F2)

The following is an example of a protocol for preparing human platelet releasate (releasate F2) in accordance with the current disclosure. This example protocol does not include a step of centrifuging platelet rich plasma.
1. Units of (Platelet Rich Plasma) PRP were weighed.
2. The following parameters or test results were recorded for each unit: ABO-type, blood cell count and infectious disease marker (IDM).
3. Only O-type (Rh+ and Rh−) PRP were used in this example but any ABO blood type can be used.
4. PRP units were adjusted to a final volume of 250 ml per bag.
5. The bags containing PRP were first frozen to −55° C. to −80° C. using the rapid cooling freezer.
6. The bags containing PRP were then transferred to −80° C. freezer for storage.
   NOTE: PRP units were collected, processed and stored at −80° C. until a critical manufacturing volume was attained. This storage facilitates accumulation of PRP units for large-scale manufacturing.
7. The bags containing PRP were removed from the −80° C. freezer and thawed for 30 min using a water bath set at 37° C.
   NOTE: Steps 5, 6 and 7 may be skipped upon abundant availability of PRP units at step 1 for large-scale manufacturing
8. The platelets were activated by the injection of a sterile calcium chloride solution at a final concentration of 40 mM using sterile-welded connections.
9. The contents of the bag were agitated by placing the units on an orbital shaker set-up at 38° C.-40° C., and 250 rpm for 90 min.
10. Following the agitation step, the calcium chloride-treated PRP units were balanced in a centrifuge set for 4000 g at 4° C.
11. The units were centrifuged for 15 min.
12. A new collection bag system was assembled by sterile welding/connecting a 1 L transfer bag to a 170-260 micron filter system. For e.g. Fenwal y-type blood component recipient set containing 170-260 micron filter. This transfer bag assembly will be used in the next step to collect and pool the human platelet releasate.
13. Using the extractor system, the supernatant (referred to as human platelet releasate) in every bag was transferred into the collection bag system.
14. At 250 mL of human platelet releasate per unit, 4 units were pooled to a maximum volume of 1000 ml into the 2-liter transfer bag of the collection bag system.
15. The pooled unit were then stored in 2 L bag containing 1000 ml human platelet releasate at −80° C. overnight.
16. After overnight treatment at −80° C., the human platelet releasate was thawed for 30 min in a 37° C. water bath
17. The thawed human platelet releasate was connected to a leukoreduction system using a sterile tube welder. The filtered human platelet releasate was collected in the attached 2 L transfer bag
18. Further, aseptic filtration of the human platelet releasate was performed using a 0.65 um in-line filter (e.g. Macopharma in-line filter) that was attached to the 2 L transfer bag containing the product
19. After filtration, aliquots of 50 ml of filtered human platelet releasate were transferred into 60 ml storage bottles.
20. The storage bottles (20 bottles for every 1000 ml) were transferred to a freezer set at or −80° C.

### Example 10—Differential Biomarker Expression in Platelet Releasate

An antibody array analysis was performed on various compositions: Releasate F1 and Releasate F2, human AB serum, FBS, and three commercial platelet lysates (Lysate A, B, and C). For antibody array analysis, all samples were processed as a service by Raybiotech (Norcross, Ga.) using the Quantibody Human Cytokine Antibody Array 4000. This quantitative array provides the concentration of 200 human growth factors, cytokines, chemokines, and other factors simultaneously (see Table 1). All experiments were conducted following the recommended manufacturer's instructions as provided in the antibody array package insert. Briefly, after 30 min of incubation with blocking buffer, 100β L of twofold-diluted samples were added to each well of the glass slide. After the prescribed incubation period and extensive washing, the biotin-labeled detection antibody and detection antibody cocktail were added for 1-2 hours (room temperature) and then washed. Cy3 equivalent dye-conjugated streptavidin was then added and incubated for 1 h at room temperature. The slide was thoroughly washed prior to scanning using a microarray scanner. Each protein had a standard curve included with measurements from known dilutions of purified standard protein. Each slide array contained positive control samples that were used for normalization purposes. Measurements were based on fluorescent intensity of bound labeled antibodies to each spot and calculated from the mean of four spots/antibody. Slides were measured, analyzed, and normalized to positive controls using Raybiotech software. Final results were expressed as picograms of protein/ml extract.

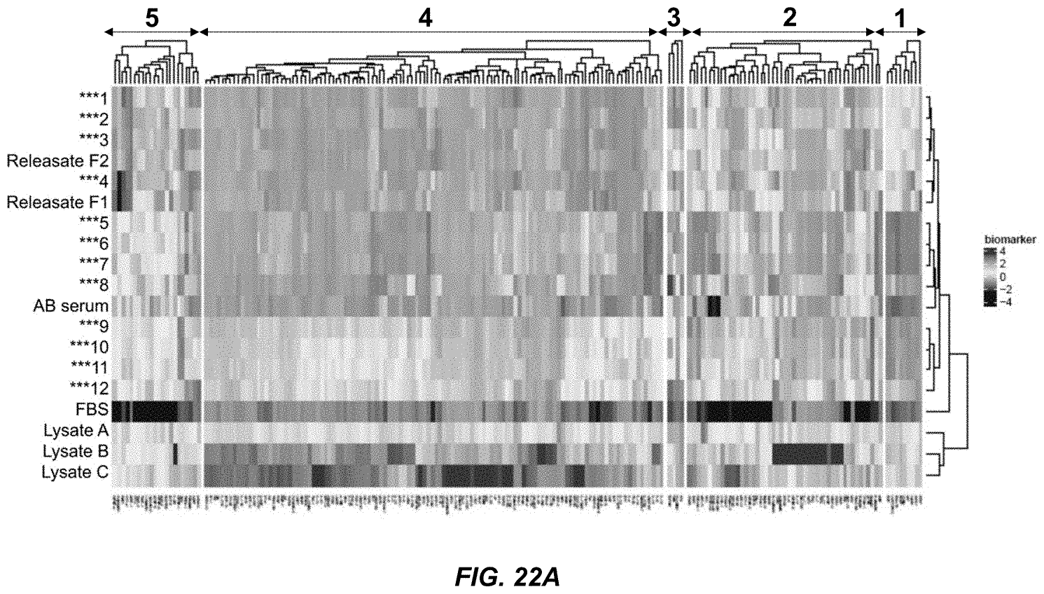

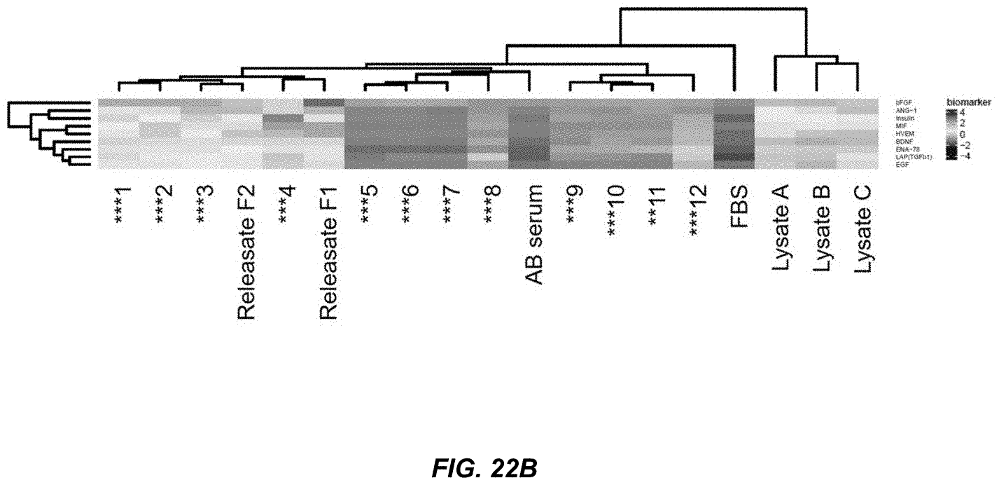

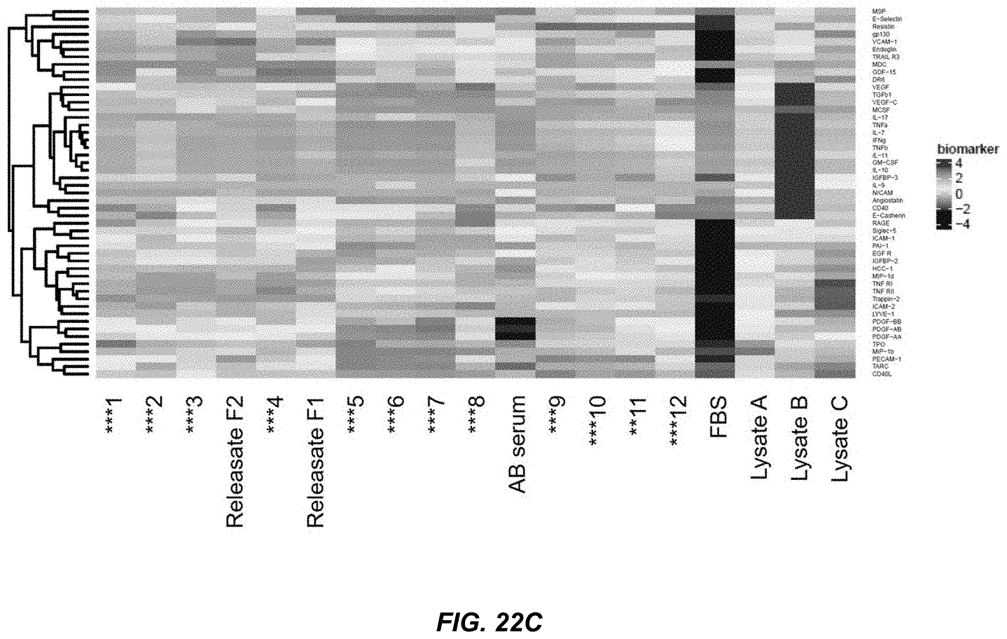

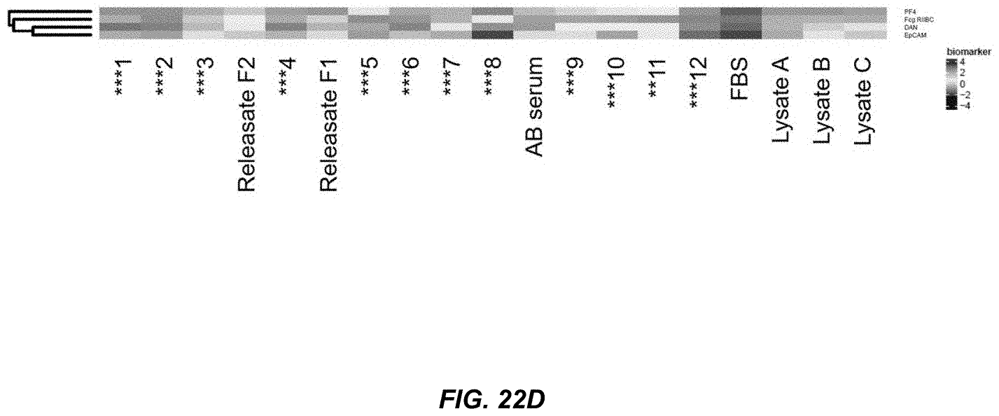

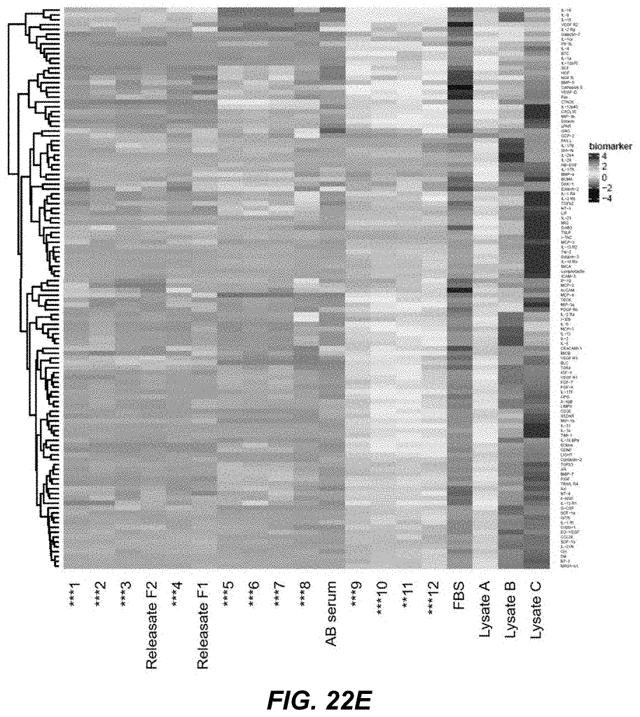

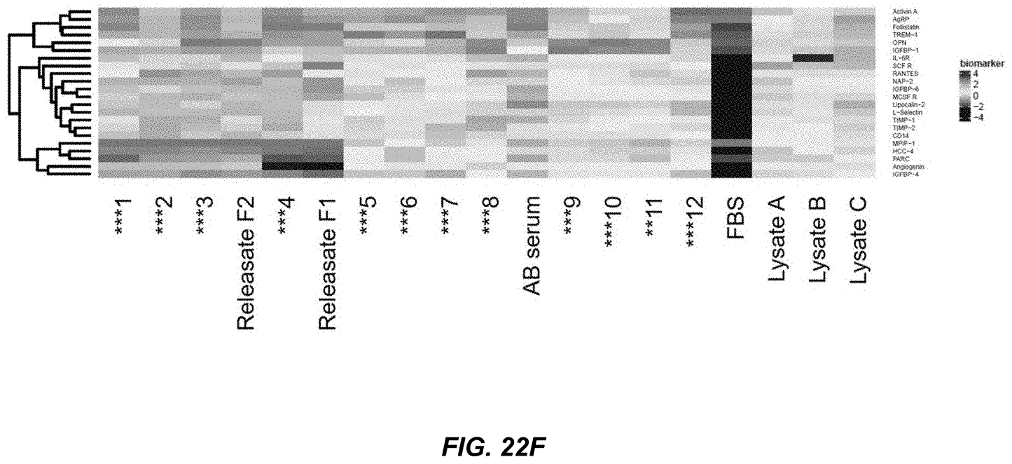

The results of the antibody array analysis are shown in Table 1 and FIGS. 22A-22F, and demonstrate differential biomarker expression in the releasate compared with the other samples. FIG. 22A shows levels of all proteins in all samples, separated into five groups (1-5) as indicated. FIGS. 22B-22F show protein levels for each of the five groups shown in FIG. 22A. Values labeled 0.0 in Table 1 were below the limit of detection (LOD). Values in italics in Table 1 were above the highest standard for the assay (MAX).

TABLE 1

| | Protein/ | FBS | Lysate A | Lysate B | Lysate C | AB serum | Releasate | Releasate |
|---|---|---|---|---|---|---|---|---|
| LOD/MAX | Biomarker | (pg/ml) | (pg/ml) | (pg/ml) | (pg/ml) | (pg/ml) | Fl (pg/ml) | F2 (pg/ml) |
| 23.88/8888.9 | 6Ckine | 0.00 | 977.06 | 1503.50 | 1707.74 | 0.00 | 0.00 | 0.00 |
| 15.85/8000 | Axl | 0.00 | 2243.43 | 2712.52 | 3689.25 | 353.95 | 606.44 | 760.18 |
| 61.55/40000 | BTC | 0.00 | 692.14 | 658.41 | 1074.65 | 0.00 | 0.00 | 0.00 |
| 12.12/80000 | CCL28 | 0.00 | 941.36 | 1569.60 | 1671.52 | 18.67 | 141.77 | 93.39 |
| 39.81/100000 | CTACK | 0.00 | 1422.41 | 2831.24 | 2890.65 | 394.50 | 119.13 | 36.27 |
| 17.29/40000 | CXCL16 | 0.00 | 9151.20 | 12326.36 | 19133.38 | 3786.85 | 2085.68 | 3406.45 |
| 6.06/20000 | ENA-78 | 0.00 | 2673.43 | 2773.23 | 2714.33 | 73.27 | 2242.52 | 2023.85 |
| 222.07/40000 | Eotaxin-3 | 0.00 | 28225.52 | 38632.71 | 63436.67 | 0.00 | 516.17 | 59.69 |
| 1.32/20000 | GCP-2 | 0.24 | 203.53 | 186.81 | 202.12 | 1.65 | 49.77 | 40.06 |
| 1.86/2000 | GRO | 10.26 | 614.38 | 700.99 | 694.33 | 20.59 | 277.61 | 170.04 |
| 5.37/8000 | HCC-1 | 0.00 | 2368.54 | 3022.26 | 3509.02 | 1446.93 | 1624.91 | 1823.39 |
| 3.62/20000 | HCC-4 | 0.00 | 501.38 | 642.12 | 542.90 | 554.95 | 218.77 | 300.54 |
| 1780.95/40000 0 | IL-9 | 0.00 | 431.37 | 7348.47 | 1487.15 | 0.00 | 0.00 | 0.00 |
| 10.92/200000 | IL-17F | 0.00 | 9499.96 | 30539.55 | 33967.84 | 1247.06 | 2114.80 | 1627.28 |
| 133.69/120000 | IL-18 BPa | 3.09 | 4043.81 | 10358.98 | 9082.24 | 288.64 | 528.17 | 285.55 |
| 5.67/20000 | IL-28A | 0.00 | 118.96 | 292.42 | 210.41 | 0.00 | 0.00 | 0.00 |
| 489.62/200000 | IL-29 | 0.00 | 2454.73 | 5801.69 | 4237.24 | 0.00 | 0.00 | 0.00 |
| 2.05/80000 | IL-31 | 3.66 | 1147.03 | 1723.44 | 2949.69 | 77.67 | 173.21 | 128.61 |
| 10.17/20000 | IP-10 | 0.00 | 1760.35 | 1739.18 | 2039.11 | 393.79 | 540.40 | 290.90 |
| 20.59/20000 | I-TAC | 0.00 | 1325.01 | 2289.52 | 3392.36 | 0.00 | 120.06 | 88.29 |
| 48.85/26000 | LIF | 0.00 | 873.62 | 1232.70 | 1814.24 | 58.07 | 0.00 | 56.71 |
| 14.02/20000 | LIGHT | 0.00 | 1050.96 | 1650.76 | 1814.39 | 47.04 | 41.48 | 25.18 |
| 58.16/200000 | Lymphotactin | 4.17 | 1226.77 | 1447.06 | 2320.52 | 0.00 | 28.54 | 46.08 |
| 2.22/4000 | MCP-2 | 0.00 | 447.49 | 599.79 | 628.14 | 173.72 | 137.23 | 29.38 |
| 2.57/8000 | MCP-3 | 3.00 | 183.25 | 226.18 | 322.94 | 2.56 | 55.20 | 5.03 |
| 3.79/20000 | MCP-4 | 0.00 | 194.93 | 203.40 | 255.01 | 17.73 | 95.24 | 42.15 |
| 1.66/20000 | MDC | 1.12 | 291.08 | 350.39 | 324.51 | 225.67 | 0.91 | 18.99 |
| 1.39/8000 | MIF | 0.14 | 822.06 | 765.95 | 809.05 | 13.69 | 1258.49 | 564.23 |
| 2.43/8000 | MIP-3a | 0.69 | 334.38 | 305.45 | 636.51 | 29.92 | 107.49 | 30.96 |
| 1.12/40000 | MIP-3b | 3.41 | 784.45 | 1386.89 | 2344.42 | 168.72 | 165.79 | 137.52 |
| 17.2/20000 | MPIF-1 | 0.00 | 704.03 | 716.10 | 793.43 | 302.83 | 0.00 | 0.00 |
| 132.92/200000 | MSP | 0.00 | 12306.20 | 8837.70 | 12579.28 | 16757.30 | 672.30 | 3230.74 |
| 4.02/8000 | NAP-2 | 0.00 | 94.93 | 113.90 | 113.35 | 100.48 | 78.53 | 82.60 |
| 63.08/200000 | OPN | 25.18 | 1742.22 | 2697.55 | 3080.63 | 1575.33 | 933.03 | 443.66 |
| 10.58/8000 | PARC | 0.00 | 1153.39 | 1138.96 | 1522.58 | 819.29 | 173.84 | 859.44 |
| 284.9/200000 | PF4 | 0.00 | 16380.54 | 16057.47 | 19183.50 | 67135.85 | 16361.42 | 32125.11 |
| 4.19/20000 | SDF-la | 0.00 | 188.57 | 325.54 | 300.07 | 11.39 | 26.85 | 10.58 |
| 6.2/20000 | TARC | 0.00 | 1205.56 | 1594.81 | 1851.81 | 25.73 | 1040.92 | 586.60 |
| 42.15/200000 | TECK | 29.16 | 2354.57 | 2972.64 | 5055.38 | 87.49 | 231.14 | 259.90 |
| 0.22/20000 | TSLP | 13.43 | 592.86 | 1013.28 | 1979.23 | 96.17 | 190.40 | 128.87 |
| 12.13/7407.4 | Activin A | 0.00 | 232.82 | 335.99 | 513.81 | 76.12 | 182.01 | 223.06 |
| 47.43/20000 | AgRP | 0.00 | 711.95 | 551.65 | 1340.30 | 77.02 | 183.32 | 355.04 |
| 9.38/4000 | Angiogenin | 0.00 | 6427.26 | 6562.00 | 6929.04 | 6564.08 | 542.48 | 5026.25 |
| 28.09/80000 | ANG-1 | 5555.35 | 51695.06 | 41108.37 | 29450.34 | 6980.71 | 79546.25 | 76536.43 |
| 1840.1/2000000 | Angiostatin | 0.00 | 119982.11 | 760990.42 | 151821.60 | 53412.84 | 301362.13 | 175077.89 |
| 10.82/20000 | Cathepsin S | 0.00 | 4621.73 | 5717.06 | 6994.74 | 3267.28 | 2398.17 | 2492.08 |
| 38.93/20000 | CD40 | 0.00 | 155.02 | 574.30 | 100.12 | 144.15 | 230.80 | 143.89 |
| 11/20000 | Cripto-1 | 10.33 | 639.81 | 971.97 | 1031.55 | 43.03 | 167.18 | 209.50 |
| 48.53/80000 | DAN | 283.06 | 1851.39 | 2541.04 | 2953.20 | 1347.01 | 1897.83 | 3116.34 |
| 70.16/160000 | DKK-1 | 0.00 | 2900.63 | 3907.69 | 3659.01 | 419.90 | 1207.90 | 1190.98 |
| 197.54/160000 | E-Cadherin | 0.00 | 1083.75 | 6400.83 | 1613.57 | 968.23 | 2438.41 | 3004.23 |
| 4.58/40000 | EpCAM | 0.00 | 57.42 | 112.13 | 76.71 | 88.49 | 88.70 | 135.58 |
| 38.59/4000 | FASL | 0.00 | 4294.25 | 8750.11 | 7148.06 | 430.07 | 1065.13 | 967.75 |
| 20.37/20000 | Fcg RIIBC | 28.34 | 956.47 | 1002.75 | 903.20 | 694.57 | 1491.14 | 2045.31 |
| 63.33/80000 | Follistatin | 66.16 | 3317.12 | 3710.97 | 4676.12 | 993.39 | 990.56 | 2197.30 |
| 46.51/200000 | Galectin-7 | 118.80 | 4809.07 | 8112.54 | 11802.94 | 1374.60 | 2758.38 | 3002.51 |
| 36.88/200000 | ICAM-2 | 658.18 | 57855.23 | 65002.24 | 98579.01 | 38018.07 | 48003.87 | 36900.00 |
| 850.56/20000 | IL-13R1 | 0.00 | 23634.74 | 45039.11 | 50748.22 | 5569.37 | 11008.37 | 11466.75 |
| 561.69/40000 | IL-13R2 | 2071.06 | 95927.96 | 159798.15 | 230033.96 | 7109.26 | 18980.50 | 19983.13 |
| 113.9/80000 | IL-17B | 0.00 | 12938.70 | 30543.09 | 24125.47 | 3659.67 | 5963.97 | 6420.78 |
| 16.53/20000 | IL-2 Ra | 22.47 | 1074.41 | 1915.81 | 1455.07 | 151.36 | 173.03 | 264.97 |
| 206.09/200000 | IL-2 Rb | 366.63 | 12879.76 | 13856.38 | 27232.78 | 1340.46 | 946.26 | 1334.15 |
| 95.61/80000 | IL-23 | 0.00 | 7290.95 | 18256.24 | 27921.25 | 645.27 | 1896.02 | 2552.51 |
| 9.65/8000 | LAP(TGFb1) | 5.19 | 7749.13 | 7883.83 | 6479.62 | 717.41 | 6383.67 | 5404.96 |
| 65.5/40000 | NrCAM | 0.00 | 982.62 | 3980.84 | 1820.54 | 1142.78 | 0.00 | 326.33 |
| 51.72/80000 | PAI-1 | 0.00 | 11903.25 | 11867.37 | 12526.83 | 5537.50 | 8631.48 | 7059.76 |
| 9.26/20000 | PDGF-AB | 92.12 | 10502.67 | 13456.40 | 13556.51 | 1000.87 | 10066.56 | 9013.17 |
| 16.4/40000 | Resistin | 0.00 | 2138.81 | 3722.22 | 2835.97 | 3604.51 | 2108.09 | 2271.88 |
| 9.13/80000 | SDF- lb | 20.61 | 1352.74 | 2295.05 | 2361.35 | 101.34 | 373.13 | 292.53 |
| 1100.58/160000 | gp130 | 0.00 | 54546.55 | 51158.45 | 72118.24 | 41619.74 | 33384.91 | 31835.98 |
| 20.59/80000 | Shh-N | 13.45 | 421.83 | 939.81 | 746.03 | 91.62 | 223.62 | 178.48 |
| 20.06/20000 | Siglec-5 | 0.00 | 23765.27 | 24462.78 | 24228.23 | 23326.79 | 23242.70 | 18828.52 |
| 241.88/8000 | IL-1 R4 | 0.00 | 5118.95 | 7388.49 | 11801.37 | 215.10 | 655.46 | 786.72 |
| 333.93/80000 | TGFb2 | 0.00 | 10743.64 | 13243.57 | 24034.76 | 2066.42 | 5898.57 | 4894.35 |
| 92.87/20000 | Tie-2 | 213.71 | 10811.37 | 18280.85 | 28189.12 | 556.13 | 429.04 | 97.10 |
| 166/400000 | TPO | 91.05 | 1624.39 | 1167.67 | 1317.49 | 499.20 | 1567.43 | 802.53 |
| 36.34/16000 | TRAIL R4 | 171.28 | 6544.38 | 11320.86 | 13389.87 | 374.88 | 224.19 | 566.23 |
| 134.45/40000 | TREM-1 | 0.00 | 8021.93 | 8844.75 | 10047.34 | 3387.05 | 3059.49 | 3910.68 |
| 13.11/40000 | VEGF-C | 0.00 | 436.37 | 1017.48 | 596.71 | 216.06 | 386.21 | 250.79 |
| 164.68/80000 | VEGF R1 | 791.93 | 19478.91 | 46105.22 | 45086.43 | 2216.11 | 4322.98 | 5577.79 |
| 8.71/2222.2 | AR | 0.00 | 1148.07 | 1930.22 | 2207.67 | 114.32 | 213.65 | 207.77 |
| 1.05/4000 | BDNF | 0.00 | 2149.41 | 2349.67 | 2202.15 | 20.18 | 1780.38 | 1151.09 |
| 20.43/40000 | bFGF | 1.63 | 91.36 | 85.01 | 110.57 | 26.36 | 482.95 | 303.88 |
| 216.47/200000 | BMP-4 | 0.00 | 1291.96 | 2545.25 | 2926.83 | 455.03 | 809.23 | 457.69 |
| 4013.74/200000 | BMP-5 | 2936.95 | 111161.14 | 122719.25 | 143597.48 | 32402.67 | 30882.11 | 51946.11 |
| 30.28/80000 | BMP-7 | 0.00 | 1351.56 | 2716.09 | 3189.21 | 89.93 | 188.95 | 247.56 |
| 1.86/20000 | b-NGF | 3.09 | 134.34 | 222.07 | 267.19 | 40.86 | 59.28 | 58.69 |
| 0.1/400 | EGF | 0.00 | 45.61 | 52.80 | 41.53 | 0.67 | 39.88 | 36.43 |
| 0.85/20000 | EGFR | 48.28 | 9349.60 | 10201.45 | 12882.50 | 9034.17 | 7041.86 | 7559.86 |
| 5.99/20000 | EG-VEGF | 9.71 | 651.17 | 1238.33 | 1100.23 | 99.83 | 161.61 | 167.63 |
| 279.11/200000 | FGF-4 | 78.16 | 10745.94 | 19206.69 | 20407.05 | 1249.62 | 2054.24 | 2799.48 |
| 32.03/20000 | FGF-7 | 86.76 | 2132.34 | 4302.60 | 4207.08 | 354.00 | 457.94 | 498.06 |
| 2.31/4000 | GDF-15 | 0.00 | 108.91 | 133.37 | 113.78 | 125.50 | 42.58 | 65.10 |
| 10.72/8000 | GDNF | 13.34 | 1253.42 | 2064.76 | 2261.75 | 158.77 | 127.37 | 193.06 |
| 10.29/20000 | GH | 0.00 | 387.54 | 562.57 | 625.61 | 46.28 | 110.75 | 97.45 |
| 11.93/20000 | HB-EGF | 2.70 | 244.95 | 559.62 | 549.56 | 34.24 | 52.14 | 76.44 |
| 22.28/8000 | HGF | 31.53 | 1555.66 | 1940.21 | 2003.85 | 377.10 | 574.90 | 632.13 |
| 4.64/10000 | IGFBP-1 | 17.18 | 985.85 | 1099.84 | 1246.46 | 810.81 | 565.39 | 428.35 |
| 16.09/40000 | IGFBP-2 | 14.36 | 9717.10 | 10906.13 | 12782.38 | 7084.44 | 6929.42 | 8764.58 |
| 335.5/400000 | IGFBP-3 | 0.00 | 35641.05 | 93109.21 | 40496.00 | 36848.25 | 19764.04 | 31307.02 |
| 286.4/400000 | IGFBP-4 | 812.58 | 175210.84 | 185189.58 | 200673.90 | 148741.06 | 78954.29 | 123529.17 |
| 66.97/200000 | IGFBP-6 | 0.00 | 10466.84 | 11723.29 | 11758.75 | 8730.63 | 8065.97 | 9349.38 |
| 16.14/40000 | IGF-1 | 0.00 | 340.84 | 734.15 | 714.59 | 0.24 | 8.78 | 39.34 |
| 34.09/40000 | Insulin | 0.00 | 964.54 | 959.13 | 852.74 | 123.80 | 1066.65 | 855.51 |
| 47.37/80000 | MCSF R | 0.00 | 14405.16 | 17792.77 | 18333.13 | 13172.03 | 12480.95 | 14153.68 |
| 18.69/20000 | NGF R | 0.00 | 601.19 | 587.60 | 889.50 | 147.76 | 148.87 | 297.87 |
| 62.06/80000 | NT-3 | 0.00 | 2273.88 | 3435.84 | 4593.04 | 444.71 | 227.49 | 572.85 |
| 31.34/20000 | NT-4 | 0.00 | 904.32 | 1342.84 | 1529.75 | 314.04 | 187.40 | 397.59 |
| 18.31/8000 | OPG | 10.71 | 1019.57 | 2349.99 | 2797.91 | 240.34 | 386.79 | 540.76 |
| 3.89/20000 | PDGF-AA | 2.10 | 1013.32 | 1336.29 | 1094.16 | 177.13 | 936.95 | 1104.68 |
| 5.56/8000 | PIGF | 0.00 | 439.31 | 769.67 | 1022.39 | 83.24 | 35.78 | 73.11 |
| 7.58/20000 | SCF | 43.88 | 1161.23 | 1682.15 | 2126.99 | 185.07 | 262.83 | 401.11 |
| 24.29/40000 | SCF R | 1.98 | 2446.05 | 2782.92 | 2668.39 | 3053.31 | 2093.23 | 3115.82 |
| 0.16/20000 | TGFa | 2.16 | 42.33 | 108.05 | 102.96 | 13.65 | 24.60 | 33.60 |
| 6196.49/200000 | TGFb1 | 0.00 | 67409.16 | 200149.82 | 117310.18 | 22411.66 | 22100.79 | 45369.89 |
| 14.59/80000 | TGFb3 | 36.17 | 995.51 | 1863.69 | 2205.80 | 167.99 | 151.39 | 220.12 |
| 10.19/20000 | VEGF | 0.00 | 190.53 | 387.83 | 208.03 | 117.90 | 140.19 | 115.36 |
| 61.35/20000 | VEGF R2 | 0.00 | 6723.91 | 6926.63 | 7261.36 | 3473.25 | 2945.24 | 3923.43 |
| 27.83/80000 | VEGF R3 | 28.76 | 2162.60 | 3298.92 | 3826.34 | 563.59 | 803.60 | 1296.15 |
| 5.31/40000 | VEGF-D | 10.15 | 857.82 | 907.84 | 1177.51 | 351.47 | 212.59 | 436.20 |
| 1.49/148.1 | BLC | 0.00 | 330.54 | 597.14 | 719.63 | 15.00 | 164.91 | 157.03 |
| 3.7/8000 | Eotaxin | 0.00 | 152.88 | 158.95 | 293.53 | 20.25 | 15.15 | 19.38 |
| 9.94/2000 | Eotaxin-2 | 5.92 | 1140.51 | 1160.02 | 1314.10 | 629.30 | 521.48 | 372.61 |
| 9.41/40000 | G-CSF | 0.00 | 139.79 | 316.84 | 288.75 | 0.00 | 27.68 | 25.61 |
| 4.74/2000 | GM-CSF | 0.00 | 550.25 | 1089.48 | 604.47 | 2.45 | 72.22 | 54.17 |
| 9.47/8000 | I-309 | 0.00 | 169.82 | 380.26 | 301.40 | 2.24 | 11.81 | 33.16 |
| 587.46/200000 | ICAM-1 | 0.00 | 15024.53 | 14761.06 | 15404.94 | 14683.19 | 13252.92 | 12277.10 |
| 10.53/4000 | IFNg | 0.00 | 862.13 | 1797.32 | 996.82 | 0.00 | 113.22 | 88.90 |
| 11.59/4000 | IL-la | 0.00 | 278.92 | 479.90 | 552.25 | 0.00 | 24.32 | 15.43 |
| 2.99/2000 | IL-lb | 2.29 | 229.21 | 423.48 | 654.40 | 0.63 | 6.73 | 8.10 |
| 2.89/4000 | IL-lra | 0.00 | 77.82 | 110.38 | 146.89 | 6.54 | 12.04 | 7.46 |
| 15.65/4000 | IL-2 | 0.00 | 916.98 | 1808.57 | 1145.61 | 1.85 | 111.85 | 72.53 |
| 20.52/4000 | IL-4 | 0.00 | 324.51 | 835.71 | 1256.12 | 0.00 | 0.00 | 0.00 |
| 9.33/8000 | IL-5 | 0.00 | 924.12 | 1780.45 | 1197.68 | 1.01 | 110.78 | 68.62 |
| 12.85/4000 | IL-6 | 0.00 | 252.35 | 514.44 | 304.15 | 5.99 | 39.02 | 26.76 |
| 17.02/20000 | IL-6R | 0.00 | 4007.56 | 1271.14 | 5512.66 | 3159.63 | 3518.03 | 3279.38 |
| 6.92/8000 | IL-7 | 0.00 | 381.73 | 743.37 | 444.00 | 2.26 | 85.60 | 62.96 |
| 1.58/1000 | IL-8 | 0.00 | 73.91 | 230.27 | 153.92 | 13.15 | 70.01 | 69.26 |
| 0.71/8000 | IL-10 | 0.00 | 75.41 | 174.59 | 93.94 | 1.64 | 10.03 | 6.64 |
| 15.88/40000 | IL-11 | 0.00 | 99.59 | 262.33 | 101.29 | 6.27 | 28.24 | 6.61 |
| 1.31/20000 | IL-12p40 | 0.00 | 306.70 | 433.43 | 865.73 | 28.81 | 46.81 | 69.57 |
| 0.23/1000 | IL-12p70 | 0.05 | 11.79 | 22.15 | 23.12 | 0.12 | 1.62 | 1.09 |
| 1.02/2000 | IL-13 | 0.00 | 64.60 | 138.66 | 97.27 | 0.23 | 7.55 | 6.13 |
| 5.64/8000 | IL-15 | 0.00 | 522.43 | 1836.45 | 1408.00 | 9.49 | 441.34 | 461.45 |
| 4.43/10000 | IL-16 | 0.00 | 276.86 | 781.19 | 753.49 | 19.78 | 408.41 | 263.53 |
| 2.88/8000 | IL-17 | 0.00 | 73.60 | 158.95 | 88.19 | 0.00 | 24.51 | 10.24 |
| 28.85/4000 | MCP-1 | 0.00 | 2552.19 | 4431.17 | 2465.79 | 719.81 | 703.74 | 289.40 |
| 3.93/8000 | MCSF | 0.00 | 177.22 | 294.61 | 220.80 | 8.86 | 51.81 | 50.46 |
| 3.72/10000 | MIG | 15.03 | 1216.55 | 2082.62 | 4117.41 | 65.17 | 69.82 | 58.98 |
| 1.47/20000 | MIP-la | 2.64 | 332.69 | 489.86 | 688.89 | 39.96 | 106.20 | 82.87 |
| 0.86/2000 | MIP-lb | 0.02 | 71.25 | 47.86 | 52.82 | 21.36 | 44.28 | 31.34 |
| 2.03/20000 | MIP-id | 0.68 | 1534.66 | 1446.43 | 1792.11 | 918.55 | 1056.74 | 1067.84 |
| 0.68/4000 | PDGF-BB | 13.37 | 3028.84 | 3395.10 | 4169.16 | 270.25 | 3247.88 | 3125.74 |
| 11.07/40000 | RANTES | 159.47 | 2931.21 | 2893.61 | 2917.90 | 2485.06 | 3185.91 | 2531.42 |
| 19.08/80000 | TIMP-1 | 4.96 | 3845.70 | 4241.69 | 4335.49 | 3149.75 | 3833.56 | 3691.75 |
| 25.34/80000 | TIMP-2 | 15.51 | 18378.58 | 17847.77 | 20378.50 | 16812.67 | 17010.44 | 16000.25 |
| 21.71/4000 | TNFa | 0.00 | 1710.12 | 3547.87 | 1862.16 | 35.24 | 388.19 | 236.06 |
| 50.81/40000 | TNFb | 0.00 | 5882.21 | 11915.76 | 6433.04 | 0.00 | 1021.51 | 646.71 |
| 22.22/80000 | TNF RI | 49.45 | 7570.85 | 8416.78 | 12870.74 | 4713.84 | 3829.86 | 4134.15 |
| 9.65/80000 | TNF RII | 44.81 | 9348.82 | 12817.33 | 17160.61 | 6956.32 | 5849.24 | 5524.72 |
| 7.69/6666.7 | 4-1BB | 56.82 | 4739.82 | 13582.39 | 14481.92 | 2235.12 | 2342.91 | 1068.69 |
| 28.13/20000 | ALCAM | 0.00 | 4850.38 | 5482.11 | 7415.80 | 3089.69 | 2392.04 | 1746.58 |
| 15.92/20000 | B7-1 | 0.00 | 2701.87 | 5220.91 | 5964.02 | 221.88 | 365.43 | 307.29 |
| 19.99/40000 | BCMA | 23.62 | 10037.01 | 18032.24 | 22881.01 | 6608.46 | 5200.38 | 3907.37 |
| 17.54/20000 | CD14 | 0.00 | 7265.87 | 7262.73 | 8005.05 | 6947.78 | 6980.18 | 6061.16 |
| 142.26/20000 | CD30 | 0.00 | 2614.37 | 3471.32 | 4789.64 | 559.45 | 223.12 | 281.08 |
| 12.87/20000 | CD40L | 0.00 | 1596.88 | 2060.17 | 2764.31 | 809.44 | 1876.96 | 909.76 |
| 25.13/20000 | CEACAM-1 | 0.00 | 5614.22 | 7228.97 | 7660.81 | 3210.50 | 2641.72 | 1625.41 |
| 1.7/8000 | DR6 | 0.00 | 6640.05 | 8585.58 | 10179.60 | 7486.35 | 3445.65 | 3792.27 |
| 102.77/40000 | Dtk | 0.00 | 38064.07 | 64224.83 | 72204.65 | 5684.73 | 6070.66 | 4379.44 |
| 4.13/8000 | Endoglin | 0.00 | 4575.73 | 4975.00 | 6640.78 | 5079.54 | 3939.07 | 3233.31 |
| 12.11/40000 | ErbB3 | 0.00 | 8412.18 | 10632.31 | 16833.27 | 2648.83 | 4958.44 | 4338.34 |
| 296.78/80000 | E-Selectin | 0.00 | 80607.26 | 81537.78 | 104789.82 | 94314.05 | 46959.79 | 41587.49 |
| 4.85/4000 | Fas | 0.00 | 2015.84 | 2398.31 | 2910.40 | 873.99 | 1065.47 | 1037.83 |
| 8.9/4000 | F1t-3L | 4.15 | 188.08 | 254.30 | 311.51 | 104.46 | 66.74 | 40.32 |
| 111.98/20000 | GITR | 0.00 | 11396.61 | 18351.40 | 20625.82 | 806.02 | 1245.90 | 836.53 |
| 33.13/80000 | HVEM | 0.00 | 9990.23 | 12176.85 | 12809.53 | 1020.62 | 14507.73 | 5559.75 |
| 16.28/200000 | ICAM-3 | 0.00 | 3042.35 | 4449.89 | 6174.90 | 402.70 | 473.49 | 142.16 |
| 58.42/200000 | Contactin-2 | 0.00 | 4117.68 | 6510.85 | 7232.12 | 878.38 | 981.75 | 494.01 |
| 2.99/8000 | IL-1 RI | 0.00 | 376.97 | 585.53 | 614.37 | 104.70 | 87.91 | 62.87 |
| 599.63/20000 | IL-2 Rg | 50.79 | 4506.80 | 4973.59 | 5743.04 | 3762.41 | 3659.95 | 1929.44 |
| 13.06/8000 | IL-10 Rb | 0.00 | 2505.03 | 3696.69 | 6368.61 | 172.82 | 161.53 | 65.23 |
| 28.4/20000 | IL-17R | 0.00 | 1389.87 | 4387.78 | 4353.35 | 419.40 | 404.98 | 194.15 |
| 192.76/40000 | IL-21R | 0.00 | 109885.80 | 208815.22 | 205343.62 | 6169.84 | 18031.83 | 13016.43 |
| 10.68/8000 | LIMPII | 0.00 | 4849.26 | 12512.46 | 12825.16 | 1964.01 | 2436.89 | 1149.46 |
| 0.93/2000 | Lipocalin-2 | 0.66 | 844.19 | 972.78 | 1167.01 | 593.26 | 843.81 | 727.84 |
| 241.92/200000 | L-Selectin | 0.00 | 68206.33 | 78067.37 | 87698.32 | 67233.70 | 69954.43 | 59901.92 |
| 4.44/4000 | LYVE-1 | 0.00 | 3403.17 | 5136.38 | 5214.27 | 2516.14 | 3169.48 | 2476.79 |
| 16.2/20000 | MICA | 10.36 | 5294.46 | 5298.86 | 10089.04 | 746.58 | 449.17 | 162.34 |
| 109.01/30000 | MICB | 0.00 | 5977.05 | 8075.53 | 7749.44 | 2241.79 | 2415.87 | 688.51 |
| 28.81/30000 | NRG1-b1 | 0.00 | 3748.80 | 7246.15 | 7918.56 | 389.07 | 792.54 | 437.23 |
| 243.16/200000 | PDGF Rb | 0.00 | 10530.91 | 10147.75 | 18547.58 | 4380.58 | 4455.19 | 2399.88 |
| 23.8/40000 | PECAM-1 | 0.00 | 4431.07 | 4745.19 | 5461.89 | 2372.60 | 3891.62 | 1880.59 |
| 3.39/20000 | RAGE | 0.00 | 777.48 | 855.80 | 1076.42 | 808.65 | 961.35 | 836.08 |
| 17.52/20000 | TIM-1 | 0.00 | 3948.78 | 7525.43 | 10881.87 | 836.20 | 1020.65 | 531.10 |
| 5.53/10000 | TRAIL R3 | 0.00 | 3549.58 | 3355.87 | 3712.54 | 2555.62 | 2568.54 | 1868.37 |
| 21.61/20000 | Trappin-2 | 0.00 | 4895.76 | 6759.80 | 8960.55 | 3193.05 | 2387.32 | 2400.34 |
| 43.75/80000 | uPAR | 0.00 | 2537.80 | 3046.89 | 4511.68 | 289.02 | 598.93 | 430.29 |
| 215.35/400000 | VCAM-1 | 550.42 | 401653.50 | 359528.59 | 402763.28 | 340474.68 | 175436.92 | 143002.98 |
| 56.18/20000 | XEDAR | 0.00 | 3550.46 | 3842.37 | 6524.56 | 648.13 | 629.55 | 283.63 |

### Example 11—MSC Doubling Time with Platelet Releasate

Bone marrow-derived MSCs (BM-MSCs) were seeded into the wells of a 24 well plate at a density of 5,000 cells per well. Growth medium consisting of DMEM (Gibco) with a 1× concentration of Glutamax (Gibco) supplemented with either 5% lysates, 10% FBS, 10% AB serum, or 5% releasate formulations were added to the wells. Commercially available lysates (5% supplement concentration) as well as FBS and AB Serum (10% supplement concentration) served as controls. Each condition, including the controls, were run in triplicate. Cells were expanded under standard cell culture conditions i.e. using a humidified, 37° C., 5% C02/95% air environment, for a period of five days. Cell proliferation was assessed on day five using the CellTiter-Glo Luminescent Cell Viability Assay (Promega, Madison, Wis.). Briefly, the media was removed, and the cells were washed three times in sterile PBS. 200 ul of the prepared CellTiter-Glo reagent was added to each of the wells which were then incubated at 37° C. for 2 minutes with agitation. Upon removal from the incubator, the well plate was allowed to sit for 10 minutes at room temperature to equilibrate the luminescent signal. The entire content of each well was then transferred to the wells of a 96-well plate that was compatible with the luminometer. The luminescence of each well was measured using a Tecan Spark plate reader (Morrisville, N.C.). The number of cells per well was determined using a standard curve of relative light units (RLU) versus known concentrations of cells run in parallel with the experimental samples. From cell number, the population doubling level (PDL) was calculated using the following equation: PDL=3.322(Log A-Log B), where A is the number of cells at the end of the growth period (per well) and B represents the initial cell number seeded into each well (in this experiment, 5000).

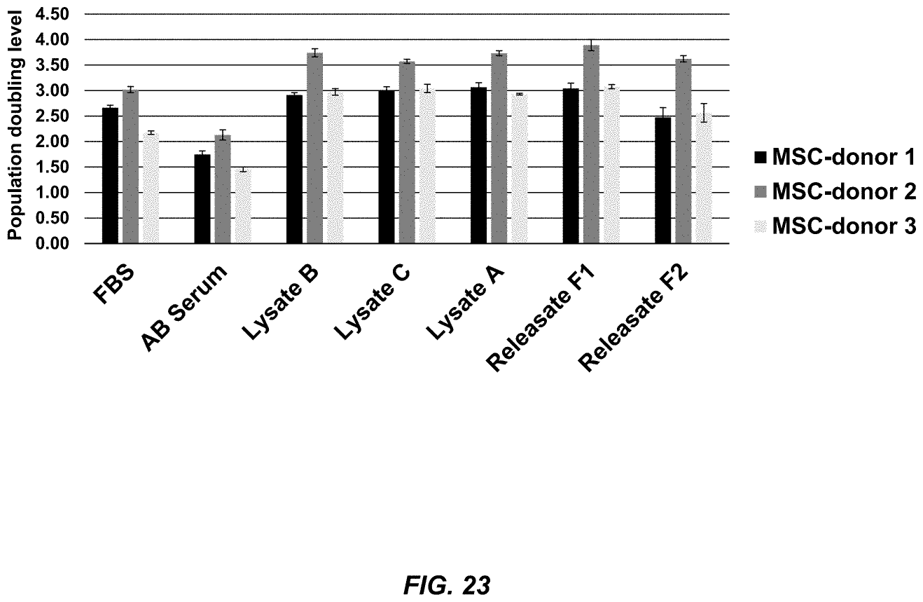

The results of the MSC doubling experiments are shown in FIG. 23.

### Example 12—Quantification of Proteins in Releasate Formulations

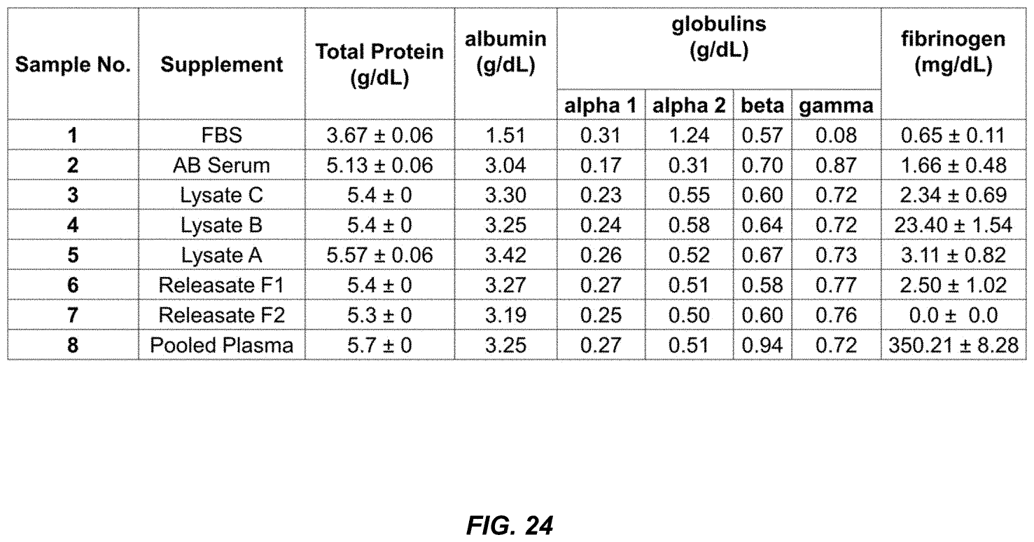

A total protein assay was performed on various additives: FBS, human AB plasma (AB serum), one of three commercial lysates (Lysate A, Lysate B, or Lysate C), one of two platelet releasate formulations (Releasate F1, Releasate F2), or pooled plasma. The total protein assay was conducted by testing samples (i.e. FBS, lysates, releasates, etc.) with the total protein reagent kit (List. No. 7D73) using an Architect C8000 system as per the instructions provided in the package insert. To quantify the concentration of fibrinogen in the samples, the fibrinogen assay kit from Diazyme (Ref #DZ768A-K) was used as per the assay procedure prescribed in the package insert. The concentration of albumin and globulins in the test samples was quantified using the capillary protein (E)6 kit from Sebia by capillary electrophoresis with the CAPILLARYS system. The results are shown in FIG. 24.

### Example 13—Electron Microscopy of Platelets

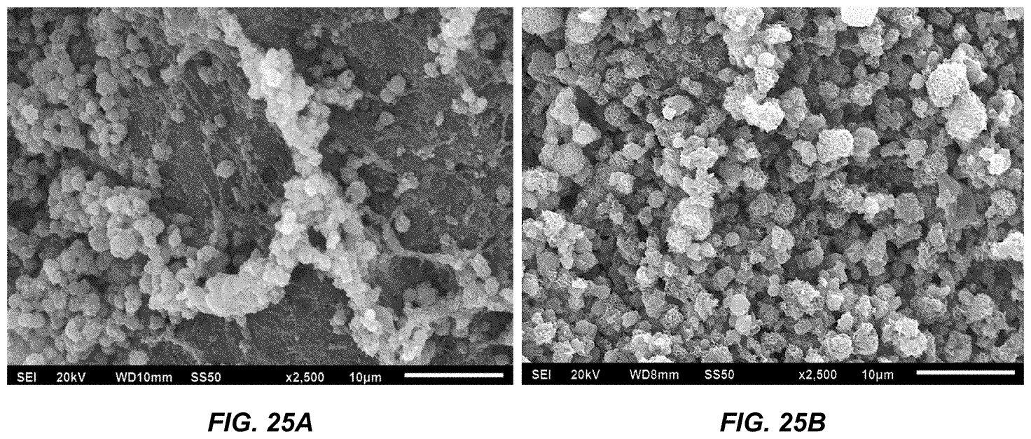

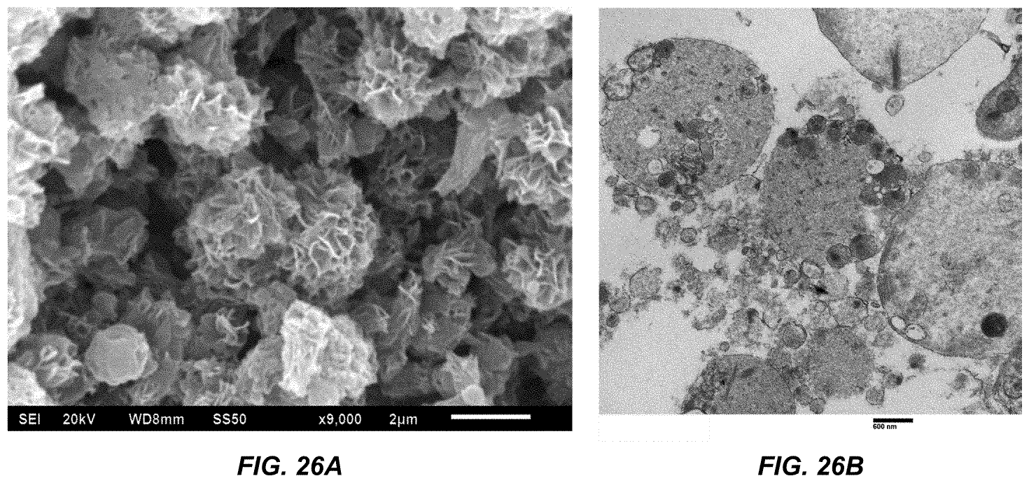

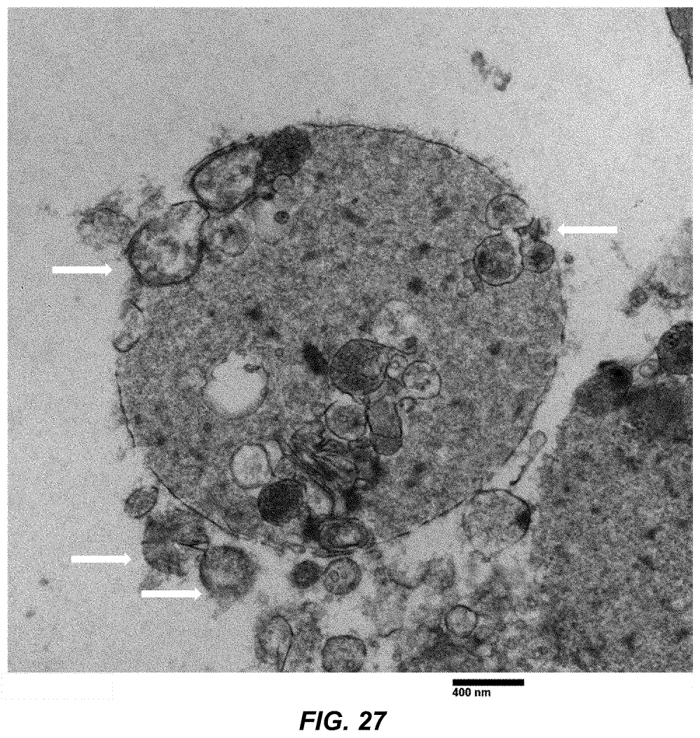

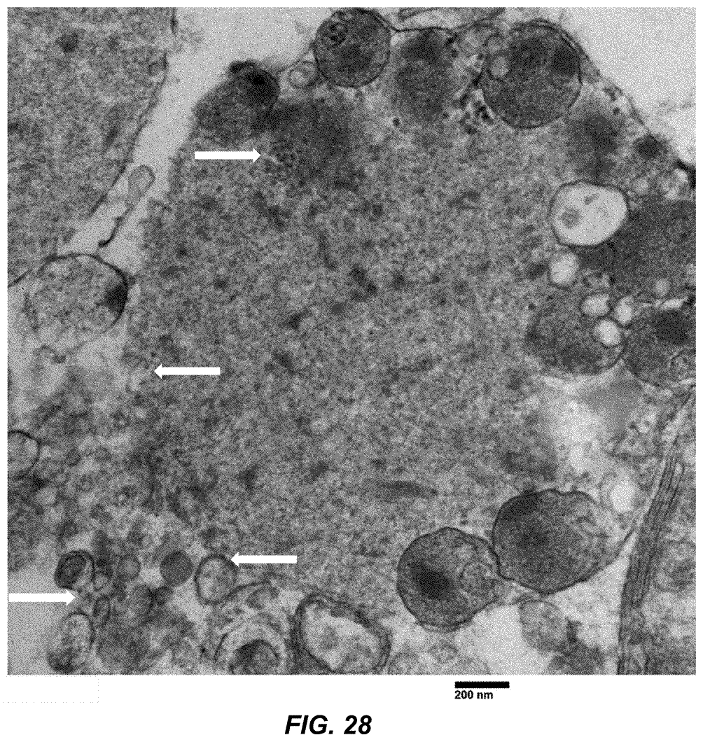

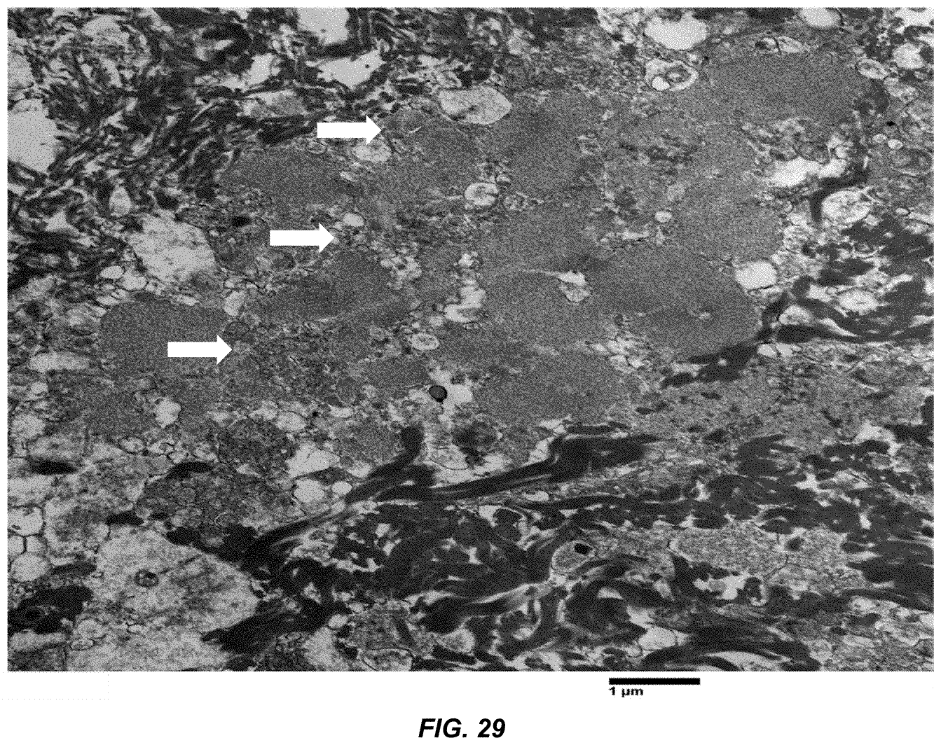

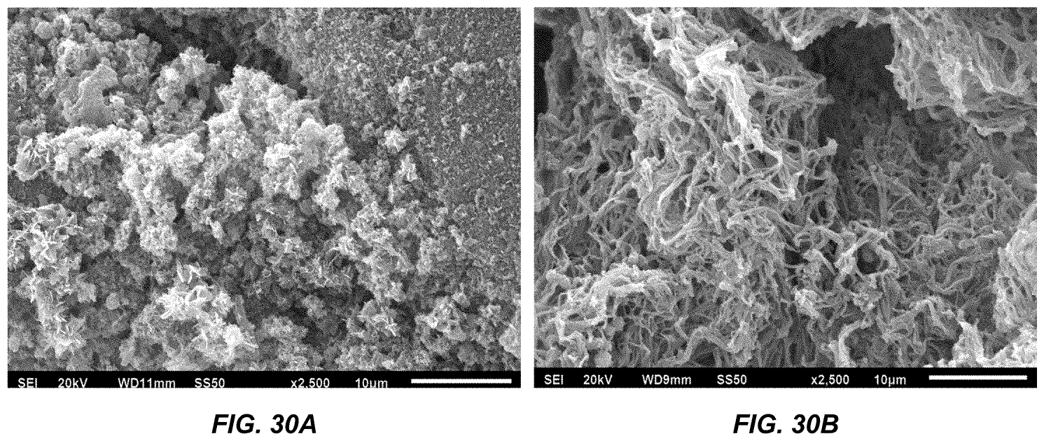

Scanning electron microscopy (SEM) and transmission electron microscopy (TEM) was performed on resting and activated platelets. For SEM, platelets from the different stages of manufacturing releasate formulations were fixed using PBS buffer containing 4% formaldehyde and 1% glutaraldehyde. After fixation, the platelets were treated with osmium tetroxide, washed using sodium cacodylate buffer, and followed by a series of washes with 20%, 40%, 60%, 80%, and 100% ethanol to dehydrate the platelets. The cells were then sputter-coated with gold-palladium alloy and stored under vacuum for 24 hours and imaged using JEOL JSM-6610LV. The transmission electron microscopy (TEM) was performed by fixing the platelets with PBS buffer containing 4% formaldehyde and 1% glutaraldehyde. After fixation, the platelets were treated with osmium tetroxide, washed using sodium cacodylate buffer, and followed by a series of washes with 20%, 40%, 60%, 80%, and 100% ethanol to dehydrate the platelets. The cells were then treated with a mixture of propylene oxide and epoxy-resin at 1:1 ratio. After incubation under vacuum for 24 hours, the cells were embedded in the 100% epoxy-resin and baked in the oven for hardening. The hardened-block containing the cells were sectioned to 90 nm thickness using an ultramicrotome with a diamond cutter and imaged using JOEL 1230. The degranulation process was observed, including vesicles and intracellular material being released from platelets into the external environment. FIGS. 25-30 show the results of these electron microscopy experiments. In particular, FIG. 29 shows platelets following degranulation, showing that the platelets are devoid of granules but remain intact (i.e., are not lysed).

### Example 14—CD Marker Expression on BM-MSCs Expanded with Releasate

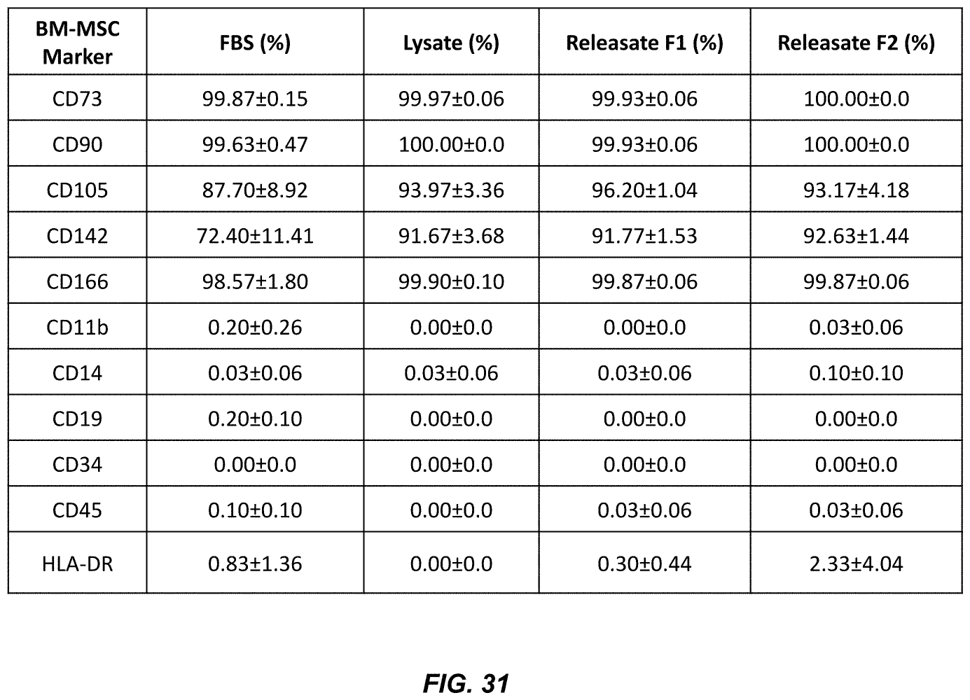

Bone marrow-derived MSCs (BM-MSCs) were expanded in basal growth medium supplemented with 10% FBS, 10% AB-serum, 5% commercial lysate (lysate A, lysate B, or lysate C), 5% releasate F1, or 5% releasate F2. MSCs used across different conditions were derived from the same donor and maninated at same PDL for all conditions, to clearly demonstrate the effect of releasate formulations on the potency of MSCs. After expansion, the cells were characterized for MSC-pertinent CD marker expression. The results are shown in FIG. 31. The CD marker expression for BM-MSCs expanded in releasate formulations (F1 and F2) met the standardized criteria for characterization of MSCs as proposed by the International Society for Cellular Therapy (ISCT).

### Example 15—Evaluation of Adipogenesis Potential

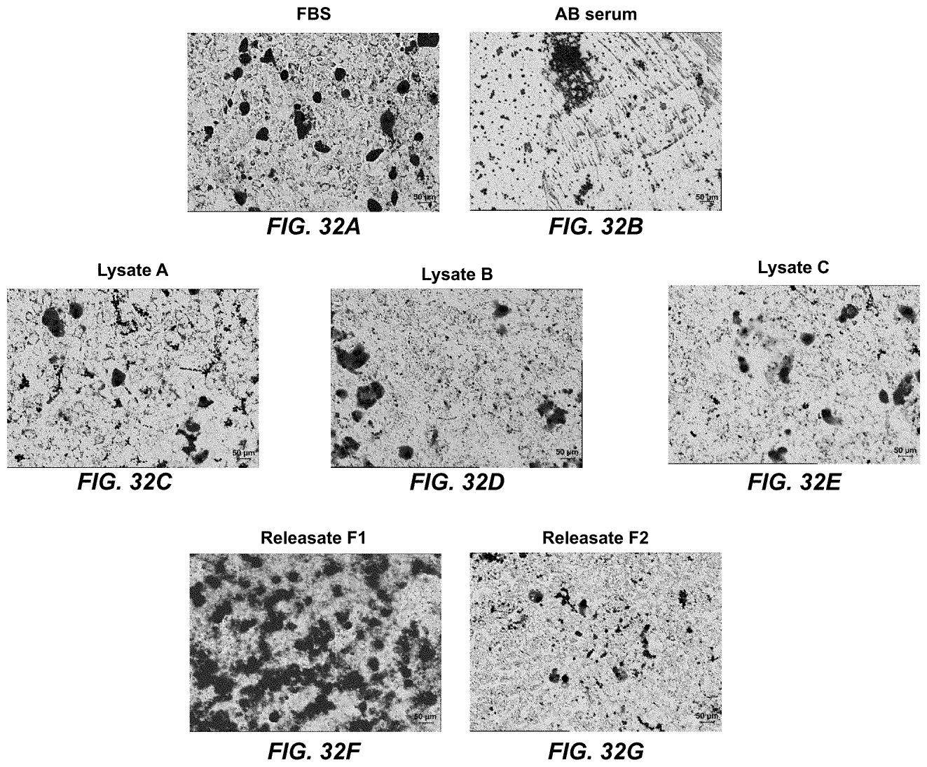

BM-MSCs were expanded in basal growth medium supplemented with 10% FBS, 10% AB-serum, 5% commercial lysate (lysate A, lysate B, or lysate C), 5% releasate F1, or 5% releasate F2. After expansion, the cells were evaluated for multipotency using differentiation medium. The BM-MSCs expanded in releasate formulations (F1 and F2) attached to tissue-culture-treated plastic, and demonstrated multipotency; meeting the standardized criteria for characterization of MSCs as proposed by the International Society for Cellular Therapy (ISCT). MSCs used across different conditions were derived from the same donor and maninated at same PDL for all conditions, to clearly demonstrate the effect of releasate formulations on the potency of MSCs. The results of this experiment are shown in FIGS. 32A-32G and suggest a preference for Releasate F1 in stimulating adipogenesis as compared with Releasate F2. The releasate formulations (F1 and F2) can also be used to evaluate potency and/or suitability of donor's cells prior to expansion (i.e. donor selection) or post cell expansion (i.e. lot or batch evaluation a.k.a. release criteria). In this context, the suitability test refers to the ability of donor's MSCs to differentiate into adipocytes.

### Example 16—Evaluation of Chondrogenesis Potential

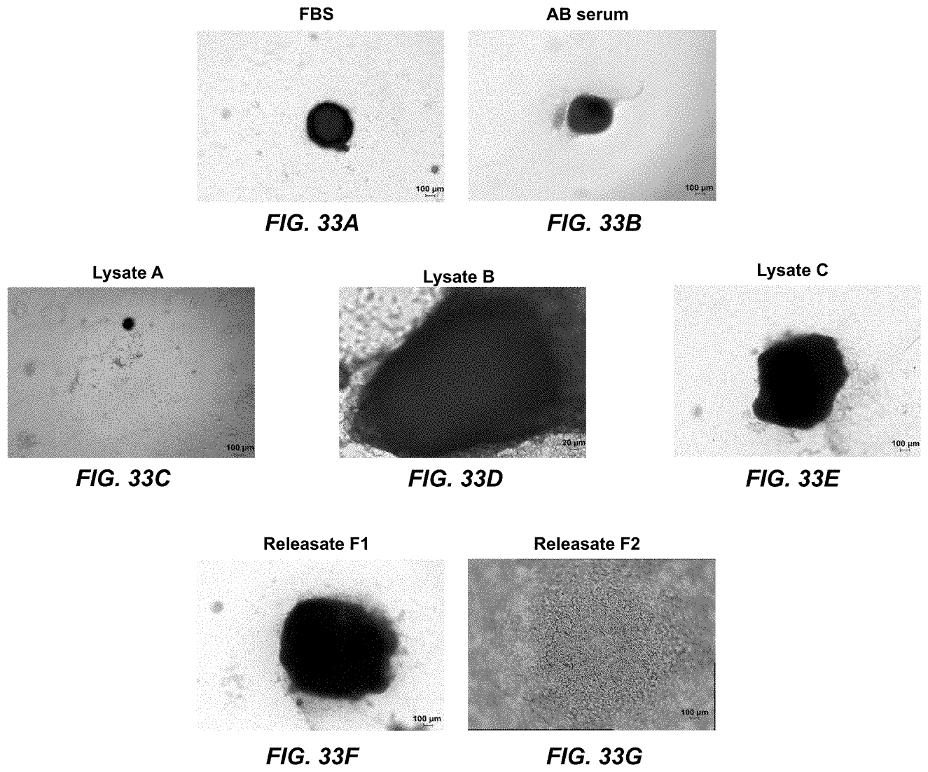

BM-MSCs were expanded in basal growth medium supplemented with 10% FBS, 10% AB-serum, 5% commercial lysate (lysate A, lysate B, or lysate C), 5% releasate F1, or 5% releasate F2. After expansion, the cells were evaluated for multipotency using differentiation medium. The BM-MSCs expanded in releasate formulations (F1 and F2) attached to tissue-culture-treated plastic, and demonstrated multipotency; meeting the standardized criteria for characterization of MSCs as proposed by the International Society for Cellular Therapy (ISCT). MSCs used across different conditions were derived from the same donor and maninated at same PDL for all conditions, to clearly demonstrate the effect of releasate formulations on the potency of MSCs. The results of this experiment are shown in FIGS. 33A-33G and suggest a preference for Releasate F1 in stimulating chondrogenesis as compared with Releasate F2. The releasate formulations (F1 and F2) can also be used to evaluate potency and/or suitability of donor's cells prior to expansion (i.e. donor selection) or post cell expansion (i.e. lot or batch evaluation a.k.a. release criteria). In this context, the suitability test refers to the ability of donor's MSCs to differentiate into chondrocytes.

### Example 17—Evaluation of Osteogenesis Potential

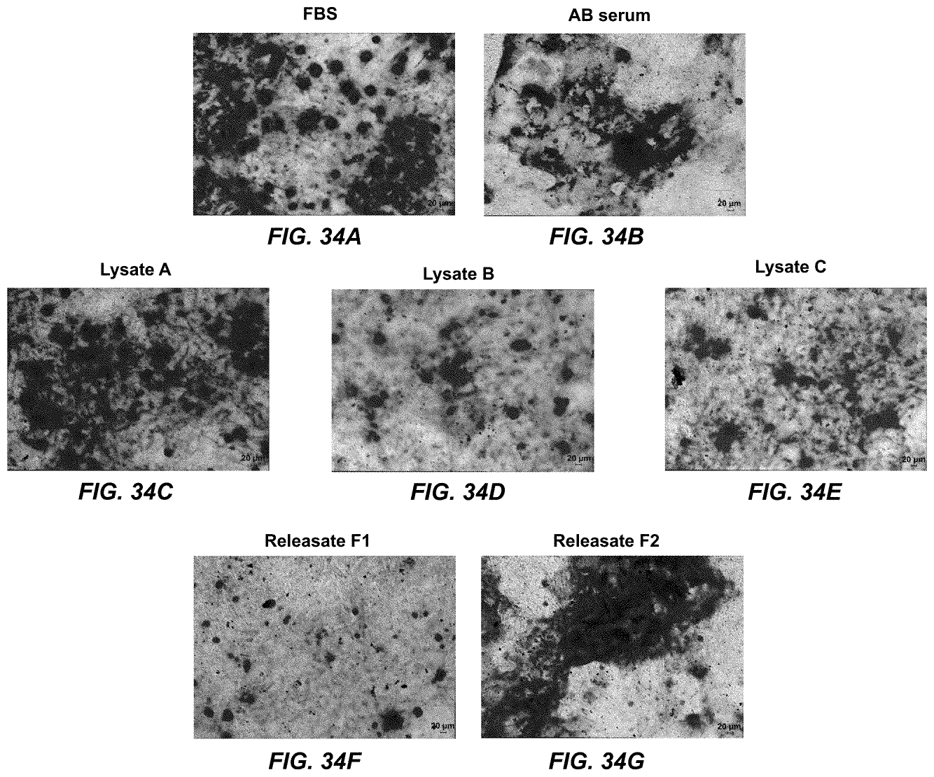

BM-MSCs were expanded in basal growth medium supplemented with 10% FBS, 10% AB-serum, 5% commercial lysate (lysate A, lysate B, or lysate C), 5% releasate F1, or 5% releasate F2. After expansion, the cells were evaluated for multipotency using differentiation medium. The BM-MSCs expanded in releasate formulations (F1 and F2) attached to tissue-culture-treated plastic, and demonstrated multipotency; meeting the standardized criteria for characterization of MSCs as proposed by the International Society for Cellular Therapy (ISCT). MSCs used across different conditions were derived from the same donor and maninated at same PDL for all conditions, to clearly demonstrate the effect of releasate formulations on the potency of MSCs. The results of this experiment are shown in FIGS. 34A-34G and suggest a preference for Releasate F2 in stimulating osteogenesis as compared with Releasate F1. The releasate formulations (F1 and F2) can also be used to evaluate potency and/or suitability of donor's cells prior to expansion (i.e. donor selection) or post cell expansion (i.e. lot or batch evaluation a.k.a. release criteria). In this context, the suitability test refers to the ability of donor's MSCs to differentiate into osteocytes.

### Example 18—Evaluation of Immunomodulation Capability of Releasate

### IDO Synthesis Assay

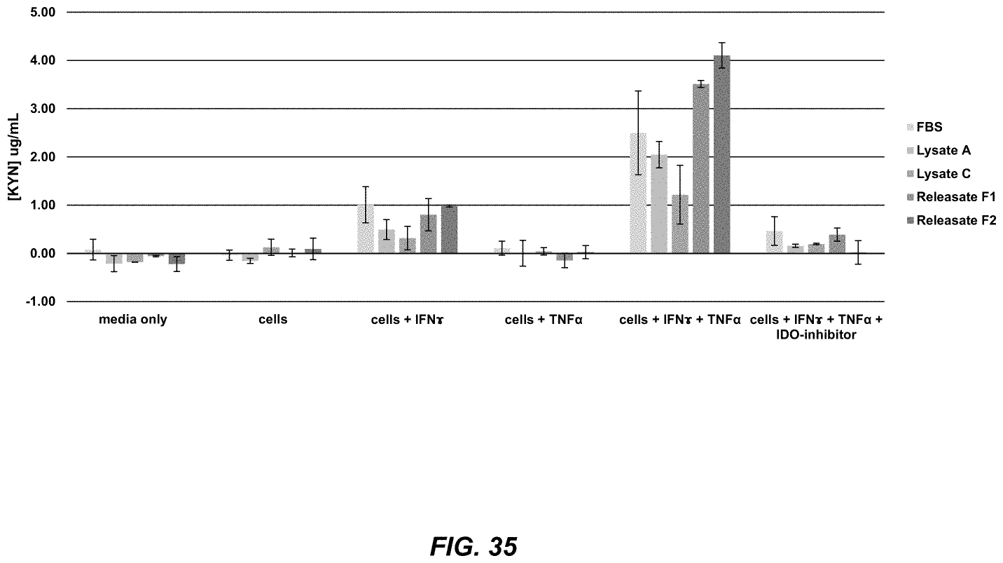

BM-MSCs were expanded in basal growth medium supplemented with 10% FBS, 10% AB-serum, 5% commercial lysate (lysate A, lysate B, or lysate C), 5% releasate F1, or 5% releasate F2. MSCs used across different conditions were derived from the same donor and maninated at same PDL for all conditions, to clearly demonstrate the effect of releasate formulations on the potency of MSCs. After expansion, the cells were evaluated for immunomodulation potential by synthesis of indoleamine 2,3-dioxygenase (IDO) in response to stimulus from the cytokines interferon gamma (IFNγ) and/or tumor necrosis factor alpha (TNFα). IDO-specific inhibitor was used as control. Cells expanded in releasate formulations demonstrated an increased production of IDO as shown in FIG. 35, where an increased IDO response led to increased kynurenine production, which was quantified in this assay. IDO acts on tryptophan, which is present in the basal growth medium, to form kynurenine (KYN), thus increased kynurenine levels are proportional to increased IDO synthesis in a cell. The releasate formulations (F1 and F2) can also be used to evaluate/test for potency and/or suitability of donor's cells prior to expansion (i.e. donor selection) or post cell expansion (i.e. lot or batch evaluation a.k.a. release criteria). In this context, the suitability/potency test refers to the ability of donor's MSCs to modulate immune response.

### PBMC Culture Assay

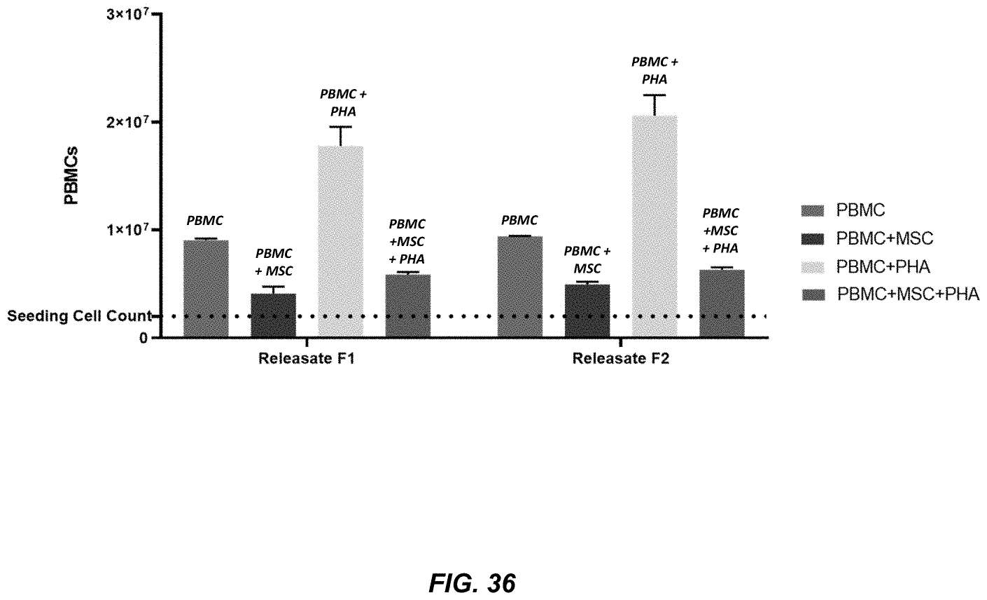

BM-MSCs were expanded in basal growth medium supplemented with 5% releasate F1 or 5% releasate F2. MSCs used across different conditions were derived from the same donor and maninated at same PDL for all conditions, to clearly demonstrate the effect of releasate formulations on the potency of MSCs. After expansion, the cells were evaluated for immunomodulation potential by co-culturing peripheral blood mononuclear cells (PBMCs) in the presence of the BM-MSCs for five days. Phytohemagglutinin (PHA) was used to trigger the expansion of the PBMCs. MSCs expanded in releasate formulations (F1 and F2) demonstrated immunomodulation properties. As shown in FIG. 36, BM-MSCs mitigated the proliferation of PBMCs. The releasate formulations (F1 and F2) can also be used to evaluate/test for potency and/or suitability of donor's cells prior to expansion (i.e. donor selection) or post cell expansion (i.e. lot or batch evaluation a.k.a. release criteria). In this context, the suitability/potency test refers to the ability of donor's MSCs to modulate immune response by mitigating the proliferation of PBMCs.

### Treg Stimulation Assay

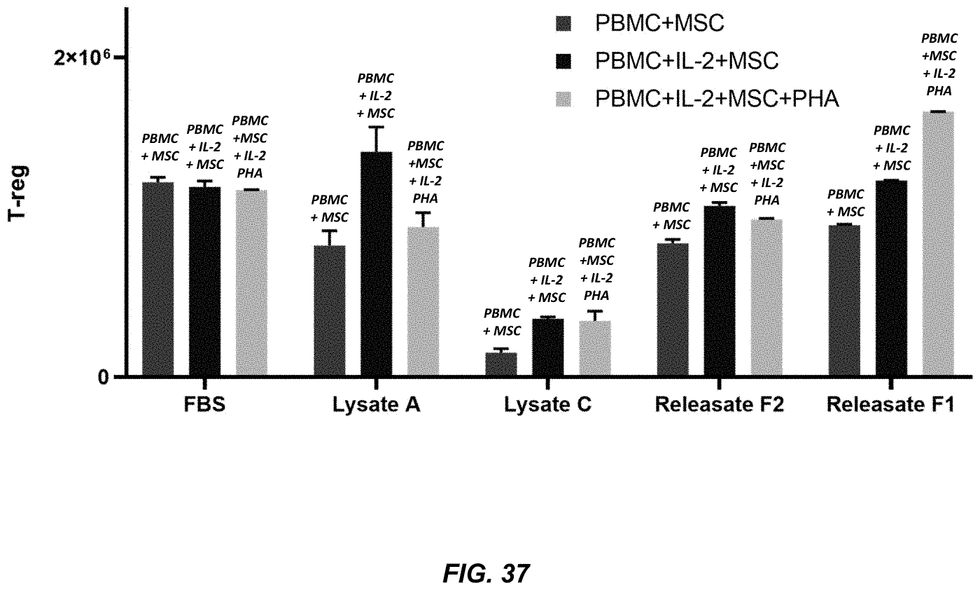

BM-MSCs were expanded in basal growth medium supplemented with 10% FBS, 10% AB-serum, 5% commercial lysate (lysate A or lysate C), 5% releasate F1, or 5% releasate F2. MSCs used across different conditions were derived from the same donor and maninated at same PDL for all conditions, to clearly demonstrate the effect of releasate formulations on the potency of MSCs. After expansion, the cells were evaluated for immunomodulation potential by co-culture of PBMCs with the BM-MSCs. Regulatory T cells (T-reg) were measured. Phytohemagglutinin (PHA) was used to trigger the expansion of PBMCs. Interleukin-1 (IL2) favors selective upregulation of regulatory T cells. As shown in FIG. 37, BM-MSCs expanded in releasate formulations (F1 and F2) demonstrated superior immunomodulation property. As shown in FIG. 37, MSCs expanded in releasate formulations do not mitigate the proliferation of T-reg. This is a desirable property in immunomodulation. The releasate formulations (F1 and F2) can also be used to evaluate/test for potency and/or suitability of donor's cells prior to expansion (i.e. donor selection) or post cell expansion (i.e. lot or batch evaluation a.k.a. release criteria). In this context, the suitability/potency test refers to the ability of donor's MSCs to modulate immune response by mitigating the proliferation of PBMCs without inhibiting the proliferation of T-reg.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references recited in the application, to the extent that they provide exemplary procedural or other details supplementary to those set forth herein, are specifically incorporated herein by reference.
Astori G, Amati E, Bambi F, Bernardi M, Chieregato K, Schafer R, et al. Platelet lysate as a substitute for animal serum for the ex-vivo expansion of mesenchymal stem/stromal cells: present and future. Stem Cell Res Ther. 2016; 7(1):93.
Baker M. Nature BSA_Reproducibility of cells. Nature. 2016; 537.
Barsotti M C, Losi P, Briganti E, Sanguinetti E, Magera A, Al Kayal T, et al. Effect of platelet lysate on human cells involved in different phases of wound healing. PLoS One. 2013; 8(12):e84753.
Bernardi M, Agostini F, Chieregato K, Amati E, Durante C, Rassu M, et al. The production method affects the efficacy of platelet derivatives to expand mesenchymal stromal cells in vitro. J Transl Med. 2017; 15(1):90.
Bieback K. Platelet lysate as replacement for fetal bovine serum in mesenchymal stromal cell cultures. Transfus Med Hemother. 2013; 40(5):326-35.
Brass E P F, W. B.; Edwards, R. V.; Lindon, O. Fibrin formation: Effect of calcium ions. Blood. 1978; 52(4).
Brass L F. Thrombin and platelet activation. CHEST. 2003; 124:18S-25S.
C. Borghese F A, C. Durante, A. Colombatti, M. Mazzucato & D. Aldinucci. Clinical-grade quality platelet-rich plasma releasate (PRP-R/SRGF) from CaCl₂)-activated platelet concentrates promoted expansion of mesenchymal stromal cells. Vox Sanguinis. 2016; 111:197-205.
C. Capelli M D, G. Borleri, P. Bellavita, R. Poma, A. Carobbio, C. Mico, A. Rambaldi, Golay and M. Introna. Human platelet lysate allows expansion and clinical grade production of mesenchymal stromal cells from small samples of bone marrow aspirates or marrow filter washouts. Bone Marrow Transplantation. 2007; 40:785-91.
Capelli C, Domenghini M, Borleri G, Bellavita P, Poma R, Carobbio A, et al. Human platelet lysate allows expansion and clinical grade production of mesenchymal stromal cells from small samples of bone marrow aspirates or marrow filter washouts. Bone Marrow Transplant. 2007; 40(8):785-91.
Castiglia S, Mareschi K, Labanca L, Lucania G, Leone M, Sanavio F, et al. Inactivated human platelet lysate with psoralen: a new perspective for mesenchymal stromal cell production in Good Manufacturing Practice conditions. Cytotherapy. 2014; 16(6):750-63.
Cavallo C, Roffi A, Grigolo B, Mariani E, Pratelli L, Merli G, et al. Platelet-Rich Plasma: The Choice of Activation Method Affects the Release of Bioactive Molecules. Biomed Res Int. 2016; 2016:6591717.
Chou M L B, T. Current methods to manufacture human platelet lysates for cell therapy and tissue engineering possible trends in product safety and standardization. International Society of Blood Transfusion, ISBT Science Series. 2017; 12:168-75.
Copland I B, Garcia M A, Waller E K, Roback J D, Galipeau J. The effect of platelet lysate fibrinogen on the functionality of MSCs in immunotherapy. Biomaterials. 2013; 34(32):7840-50.
Crespo-Diaz R, Behfar A, Butler G W, Padley D J, Sarr M G, Bartunek J, et al. Platelet lysate consisting of a natural repair proteome supports human mesenchymal stem cell proliferation and chromosomal stability. Cell Transplant. 2011; 20(6):797-811.
de Kort M, Buijsman R C, van Boeckel C A A. Synthetic heparin derivatives as new anticoagulant drugs. Drug Discovery Today. 2005; 10(11):769-79.
Eva Rubio-Azpeitia I A. Partnership between PRP vs MSC. Muscles, Ligaments and Tendons Journal. 2014; 4(1):52-62.
Fekete N, Gadelorge M, Furst D, Maurer C, Dausend J, Fleury-Cappellesso S, et al. Platelet lysate from whole blood-derived pooled platelet concentrates and apheresis-derived platelet concentrates for the isolation and expansion of human bone marrow mesenchymal stromal cells: production process, content and identification of active components. Cytotherapy. 2012; 14(5):540-54.
Fortunato T_{M}, Beltrami C, Emanueli C, De Bank P A, Pula G. Platelet lysate gel and endothelial progenitors stimulate microvascular network formation in vitro: tissue engineering implications. Sci Rep. 2016; 6:25326.
Gottipamula S, Sharma A, Krishnamurthy S, Majumdar A S, Seetharam R N. Human platelet lysate is an alternative to fetal bovine serum for large-scale expansion of bone marrow-derived mesenchymal stromal cells. Biotechnol Lett. 2012; 34(7):1367-74.
Hass R, Kasper C, Bohm S, Jacobs R. Different populations and sources of human mesenchymal stem cells (MSC): A comparison of adult and neonatal tissue-derived MSC. Cell Commun Signal. 2011; 9:12.
Heathman T R J, Stolzing A, Fabian C, Rafiq Q A, Coopman K, Nienow A W, et al. Scalability and process transfer of mesenchymal stromal cell production from monolayer to microcarrier culture using human platelet lysate. Cytotherapy. 2016; 18(4):523-35.
Hemeda H, Giebel B, Wagner W. Evaluation of human platelet lysate versus fetal bovine serum for culture of mesenchymal stromal cells. Cytotherapy. 2014; 16(2):170-80.
Hemeda H, Kalz J, Walenda G, Lohmann M, Wagner W. Heparin concentration is critical for cell culture with human platelet lysate. Cytotherapy. 2013; 15(9):1174-81.
L. L. Qiu S S L, K. L. Keeling, and R. J. Elin. Convenient and Effective Method for Removing Fibrinogen from Serum Specimens before Protein Electrophoresis. Clinical Chemistry. 2003; 49(6):868-72.
Laner-Plamberger S, Lener T, Schmid D, Streif D A, Salzer T, Oller M, et al. Mechanical fibrinogen-depletion supports heparin-free mesenchymal stem cell propagation in human platelet lysate. J Transl Med. 2015; 13:354.
Oliveira S M, Santo V E, Gomes M E, Reis R L, Mano J F. Layer-by-layer assembled cell instructive nanocoatings containing platelet lysate. Biomaterials. 2015; 48:56-65.
Pham P V V, N. B.; Pham, V. M.; Truong, N. H.; Pham, T. L.; Dang, L. T.; Nguyen, T. T.; Bui, A. N. T.; and Phan, N. K. Good manufacturing practice-compliant isolation and culture of human umbilical cord blood-derived mesenchymal stem cells. Journal of Translational Medicine. 2014; 12:56.
R. F. Silva, M. E. Alvarez, D. L. Rios, C. Lópezl, J. U. Carmona and C. MF. Rezende. Evaluation of the effect of calcium gluconate and bovine thrombin on the temporal release of transforming growth factor beta 1 and platelet-derived growth factor isoform BB from feline platelet concentrates. BMC Veterinary Research. 2012; 8:212.
Riordan N H, Madrigal M, Reneau J, de Cupeiro K, Jimenez N, Ruiz S, et al. Scalable efficient expansion of mesenchymal stem cells in xeno free media using commercially available reagents. J Transl Med. 2015; 13:232.
S. Radtke B G, W. Wagner & P. A. Horn. Platelet lysates and their role in cell therapy. ISBT Science Series. 2014; 9:193-7.
Sabrina V L, C.; Julien, B.; Sandy, E.; Pauline, B.; Thomas, B.; Francis, G.; Bruno, D. A standardized and characterized clinical grade human platelet lysate for efficient expansion of human bone marrow mesenchymal stem cells.
Shih D T, Burnouf T. Preparation, quality criteria, and properties of human blood platelet lysate supplements for ex vivo stem cell expansion. N Biotechnol. 2015; 32(1):199-211.
Strunk D, Lozano M, Marks D C, Loh Y S, Gstraunthaler G, Schennach H, et al. International Forum on GMP-grade human platelet lysate for cell propagation: summary. Vox Sang. 2018; 113(1):80-7.
Tchang L A, Pippenger B E, Todorov A, Jr., Wolf F, Burger M G, Jaquiery C, et al. Pooled thrombin-activated platelet-rich plasma: a substitute for fetal bovine serum in the engineering of osteogenic/vasculogenic grafts. J Tissue Eng Regen Med. 2017; 11(5):1542-52.
Trojahn Kolle S F, Oliveri R S, Glovinski P V, Kirchhoff M, Mathiasen A B, Elberg JJ, et al. Pooled human platelet lysate versus fetal bovine serum-investigating the proliferation rate, chromosome stability and angiogenic potential of human adipose tissue-derived stem cells intended for clinical use. Cytotherapy. 2013; 15(9):1086-97.
Wolberg A S, Campbell R A. Thrombin generation, fibrin clot formation and hemostasis. Transfus Apher Sci. 2008; 38(1):15-23.
Zulueta M M, Lin S Y, Hu Y P, Hung S C. Synthetic heparin and heparan sulfate oligosaccharides and their protein interactions. Curr Opin Chem Biol. 2013; 17(6):1023-9.

What is claimed is:

1. A method for preparing a platelet releasate, the method comprising the steps of:
i) obtaining platelets from human blood thereby obtaining platelet-rich-plasma (PRP);
ii) adding CaCl₂) to the PRP to a final concentration of greater than 25 mM, thereby generating a CaCl₂/PRP mixture; and,
iii) agitating the CaCl₂/PRP mixture for less than 6 hours thereby forming a clot and a releasate.

2. The method of claim 1, wherein the releasate comprises fibrinogen at a level of less than about 0.05 mg/dL.

3. The method of claim 1 wherein the final concentration of CaCl₂) is greater than about 30 mM.

4. The method of claim 1, wherein the final concentration of CaCl₂) is between about 25-80 mM.

5. The method of claim 1, wherein the final concentration of CaCl₂) is between about 30-50 mM.

6. The method of claim 1, wherein the final concentration of CaCl₂) is between about 35-50 mM.

7. The method of claim 1, wherein the final concentration of CaCl₂ is between about 40-47 mM.

8. The method of claim 1, wherein the final concentration of CaCl₂) is about 45 mM.

9. The method of claim 1, wherein the final concentration of CaCl₂ is about 80 mM.

10. The method of any of claims 1-9, wherein the CaCl₂/PRP mixture is agitated for less than 4 hours.

11. The method of any of claims 1-9, wherein the CaCl₂/PRP mixture is agitated for less than 180 min.

12. The method of any of claims 1-9, wherein the CaCl₂/PRP mixture is agitated for 30-150 minutes.

13. The method of any of claims 1-9, wherein the CaCl₂/PRP mixture is agitated for 45-135 minutes.

14. The method of any of claims 1-9, wherein the CaCl₂/PRP mixture is agitated from 60-90 minutes.

15. The method of any of claims 1-14, wherein the CaCl₂/PRP mixture is agitated at 50-500 rpm.

16. The method of any of claims 1-14, wherein the CaCl₂/PRP mixture is agitated at 250 rpm.

17. The method of any of claims 1-16, wherein the releasate comprises globulins, albumin, growth factors, cytokines, interleukins, interferons, chemokines, glycoproteins, fibronectin, vitronectin, or laminin.

18. The method of any of claims 1-16, wherein the releasate comprises TGF beta 1, TGF beta 3, EGF, bFGF, PDGF-AA, PDGF-BB, PDGF-AB, SDF-1α, VEGF, or HGF.

19. The method of any of claims 1-18, wherein the releasate comprises FGF-basic at a level of at least 300 pg/ml.

20. The method of any of claims 1-18, wherein the releasate comprises FGF-basic at about 300-550 pg/ml.

21. The method of any of claims 1-18, wherein the releasate comprises FGF-basic at about 350-520 pg/ml.

22. The method of any of claims 1-18, wherein the releasate comprises FGF-basic at about 400-500 pg/ml.

23. The method of any of claims 1-19, wherein the releasate comprises FGF-basic at about 450 pg/ml.

24. The method of any of claims 1-23, wherein the releasate comprises SDF-1α at about 5.0-20 pg/ml.

25. The method of any of claims 1-23, wherein the releasate comprises SDF-1α at about 7.0-15 pg/ml.

26. The method of any of claims 1-23, wherein the releasate comprises SDF-1α at about 8.0-14 pg/ml.

27. The method of any of claims 1-23, wherein the releasate comprises SDF-1α at about 9.0-12.0 pg/ml.

28. The method of any of claims 1-27, wherein the platelets were from fresh platelets, from platelets kept at room temperature, or from previously frozen platelets.

29. The method of any of claims 1-28, wherein the method further comprises the step of separating the clot from the releasate.

30. The method of any of claims 1-29, wherein the method further comprises step iv) filtering the releasate.

31. The method of claim 30, wherein step iv) comprises filtering the releasate using a filter that is between 0.45-1.0 microns.

32. The method of claim 30, wherein step iv) comprises filtering the releasate using a filter that is between 3.0-10 microns.

33. The method of claim 30, wherein step iv) comprises filtering the releasate using a filter that is between 170-260 microns.

34. The method of any of claims 1-33, wherein steps i), ii), and iii) are performed in a closed bag.

35. The method of any of claims 31-33, wherein steps i), ii), iii) and iv) are performed in a closed bag.

36. The method of any of claims 1-24, wherein steps i), ii), and iii) are performed in a closed system.

37. The method of any of claims 1-24, wherein steps i), ii), and iii) are performed on an industrial scale in a closed system.

38. The method of any of claims 31-33, wherein steps i), ii), iii) and iv) are performed on an industrial scale in a closed system.

39. The method of any of claims 1-38, wherein the entire method is performed in 4 hours or less.

40. The method of any of claim 1-27, wherein the duration of steps i), ii), and iii) is 3-4 hours.

41. The method of any of claim 1-27, wherein the duration of steps i), ii), iii) and iv) is 3-4 hours.

42. The method of any of claims 1-29, wherein the releasate is produced at a yield of about 0.2-100 liters.

43. The method of any of claims 1-29, wherein the releasate is produced at a yield of about 4.5-10 liters.

44. The method of any of claims 1-29, wherein the releasate is produced at a yield of about 50-100 liters.

45. The method of any of claims 1-44, further comprising, prior to ii), concentrating the platelets by removing excess plasma.

46. A releasate composition produced by the method of any of claims 1-45.

47. A cell culture medium comprising a releasate produced by the method of any of claims 1-45.

48. The cell culture medium of claim 47, wherein the cell culture medium is free from added heparin.

49. A method of culturing cells, the method comprising the step of expanding the cells on a cell culture medium comprising a releasate derived from a mammalian platelet-rich plasma.

50. The method of claim 49, wherein the releasate comprising fibrinogen at a level of less than about 0.05 mg/dL.

51. The method of claims 49 or 50, wherein the releasate further comprises FGF-basic at a level of at least about 300 pg/ml.

52. The method of claims 49 or 50, wherein the releasate comprises FGF-basic at about 300-550 pg/ml.

53. The method of claims 49 or 50, wherein the releasate comprises FGF-basic at about 350-520 pg/ml.

54. The method of claims 49-53, wherein the releasate comprises FGF-basic at about 450 pg/ml.

55. The method of any of claims 49-53, wherein the releasate comprises SDF-1 at about 5.0-20 pg/ml.

56. The method of any of claims 49-53, wherein the releasate comprises SDF-1 at about 7.0-15 pg/ml.

57. The method of any of claims 49-53, wherein the releasate comprises SDF-1 at about 8.0-14 pg/ml.

58. The method of any of claims 49-53, wherein the releasate comprises SDF-1 at about 9.0-12.0 pg/ml.

59. The method of any of claims 49-53, wherein the cell culture medium is free from added heparin.

60. The method of any of claims 49-53, wherein the cells are pluripotent stem cell (PSC), induced pluripotent stem cells (iPSC), bone-marrow-derived mesenchymal stromal/stem cells (BM-MSC), Adipose-derived Mesenchymal Stromal/stem Cells (ADP-MSC), T-Cells, B-cells, Natural Killer cells, Dendritic cells, Peripheral Blood-derived Mononuclear Cells, cancer cells cancer stem cells, Chinese Hamster Ovarian (CHO), umbilical cord-blood-derived cells, umbilical cord-blood tissue-derived cells, placenta-derived cells, retinal cells, neurons, fibroblasts, epithelial cells, endothelial cells or keratinocytes or fibroblasts, osteoblasts, adipocytes, chondrocytes, endothelial cells, cells of the immune system, T-cells, B-cells, NK cells, engineered cells, or neurons.

61. The method of claim 60, wherein the cells are mesenchymal stem cells.

62. The method of claim 61, wherein the releasate stimulates differentiation of the cells into osteocytes.

63. The method of any of claims 49-61, further comprising, prior to ii), concentrating the platelets by removing excess plasma.

64. The method of claim 63, wherein the releasate stimulates differentiation of the cells into chondrocytes or adipocytes.

65. The method of any of claims 49-61, wherein the releasate stimulates release of components from the cells.

66. The method claim 65, wherein the components comprise exosomes, extracellular vesicles, proteins, nucleic acids, or a combination thereof.

67. The method of claim 65 or 66, further comprising collecting the components.

68. A composition comprising a releasate from mammalian blood-derived platelets, the releasate comprising fibrinogen at a level of less than about 0.05 mg/dL.

69. The composition of claim 68, wherein the releasate is from human blood.

70. The composition of claim 68 or 69, wherein the composition is a solution.

71. The composition of claim 68 or 69, wherein the composition is a dried or lyophilized powder.

72. The composition of any of claims 68-71, wherein the releasate comprises FGF-basic at a level of at least 300 pg/ml.

73. The composition of any of claims 68-71, wherein the releasate comprises FGF-basic at about 300-550 pg/ml.

74. The composition of any of claims 68-71, wherein the releasate comprises FGF-basic at about 350-520 pg/ml.

75. The composition of any of claims 68-71, wherein the releasate comprises FGF-basic at about 400-500 pg/ml.

76. The composition of any of claims 68-71, wherein the releasate comprises FGF-basic at about 450 pg/ml.

77. The composition of any of claims 68-76, wherein the releasate comprises SDF-1 at about 5.0-20 pg/ml.

78. The composition of any of claims 68-76, wherein the releasate comprises SDF-1 at about 7.0-15 pg/ml.

79. The composition of any of claims 68-76, wherein the releasate comprises SDF-1 at about 8.0-14 pg/ml.

80. The composition of any of claims 68-76, wherein the releasate comprises SDF-1 at about 9.0-12.0 pg/ml.

81. The composition of any of claims 68-76, wherein the composition is free from added heparin.

82. The composition of any of claims 68-81, wherein the releasate comprises one or more exosomes.

83. A therapeutic formulation comprising the composition of any of claims 68-82.

84. A method of treating a mammalian subject comprising administering to the subject a composition comprising a population of stem cells, wherein the stem cells have been cultured with the releasate composition of any of claims 68-82.

85. The method of claim 84, wherein the releasate further comprises FGF-basic at a level of at least about 300 pg/ml.

86. The method of claim 84 or 85, wherein the releasate comprises SDF-1 at 5.0-20 pg/ml.

87. The method of claims 84-86, wherein the stem cells are mesenchymal stem cells.

88. The method of claims 84-86, wherein the stem cells are bone marrow-derived mesenchymal stem cells.

89. The method of claims 84-86, wherein the stem cells are, pluripotent stem cells (PSC), induced pluripotent stem cells (iPSC), bone-marrow-derived-mesenchymal stromal/stem cells (BM-MSC), Adipose-derived Mesenchymal Stromal/stem Cells (ADP-MSC), T-Cells, B-cells, Natural Killer cells, Dendritic cells, Peripheral Blood-derived Mononuclear Cells, cancer cells cancer stem cells, Chinese Hamster Ovarian (CHO), umbilical cord-blood-derived cells, umbilical cord-blood tissue-derived cells, placenta-derived cells, retinal cells, neurons, fibroblasts, epithelial cells, endothelial cells or keratinocytes.

90. The method of any of claims 84-86, wherein the population of stem cells comprises 2-100% of one type of differentiated lineage.

91. The method of any of claims 84-86, wherein the stem cells are autologous or allogenic.

92. The method of any of claims 84-86, wherein the stem cells are engineered or manipulated cells.

93. A method of treating a subject comprising administering to the subject a composition comprising stem cells, wherein the stem cells have been cultured with a releasate from human blood-derived platelets.

94. The method of claim 93, wherein the releasate comprises fibrinogen at a level of less than about 0.05 mg/dL.

95. The method of claim 93 or 94, further comprising FGF-basic at a level of at least 300 pg/ml.

96. The method of any of claim 93-95, wherein the releasate comprises SDF-1 at 5.0-20 pg/ml.

97. The method of claim 93, wherein the subject suffers from bone disease, bone defect, bone injury, osteoporosis, osteoarthrosis or spinal cord injury.

98. The method of claim 93, wherein the subject suffers from cartilage disease or cartilage defect or cartilage injury.

99. The method of claim 93, wherein the subject suffers from periodontal disease.

100. The method of claim 93, wherein the subject suffers from an autoimmune disease.

101. The method of claim 93, wherein the subject suffers from myocardial infarction.

102. The method of claim 91, wherein the subject suffers from graft versus host disease (GvHD), chronic obstructive pulmonary disease (COPD), acute respiratory distress syndrome (ARDS), poly-trauma, a systemic infection, or cancer.

103. A method for preparing a platelet releasate, the method comprising the steps of:
i) obtaining platelets from mammalian blood;
ii) adding CaCl₂) to the PRP to a final concentration of greater than 25 mM; and,
iii) agitating the CaCl₂/PRP mixture for less than 6 hours thereby forming a clot and a releasate, wherein the releasate comprises fibrinogen at a level of less than about 0.05 mg/dL.

104. The method of claim 103, wherein the mammalian blood is equine, feline, porcine, canine, bovine, chicken, feline, porcine, rabbit, dolphin, ovine, murine, rat, simian blood, from a sport animal, from a farm animal, or from a pet.

105. The method of claim 103 or 104, further comprising, prior to ii), concentrating the platelets by removing excess plasma.

106. A method of promoting cell adhesion, cell differentiation or cell expansion in tissue culture comprising coating a tissue culture container with a composition comprising a releasate from mammalian blood-derived platelets, the releasate comprising fibrinogen at a level of less than 0.05 mg/dL.

107. The method of claim 106, wherein the releasate is from human blood-derived platelets.

108. The method of claim 106 or 107, wherein the container is a petri dish, a flask or a bioreactor.

109. A method of preparing osteobiologic material, comprising a step of adding to said osteobiologic material a composition comprising a releasate from mammalian blood-derived platelets, the releasate comprising fibrinogen at a level of less than 0.05 mg/dL.

110. The method of claim 109, wherein the releasate is from human blood-derived platelets.

111. The method of claim 109 or 110, wherein the osteobiologic material is an osteobiologic graft material, a bone sponge, or a bone putty.

112. The method of claim 109 or 110, wherein the osteobiologic material further includes mammalian tissue, engineered cells or manipulated cells.

113. A method of preparing coagulation agents comprising a step of adding to said coagulation agents a composition comprising a releasate from mammalian blood-derived platelets, the releasate comprising fibrinogen at a level of less than 0.05 mg/dL.

114. The method of claim 113, wherein the releasate is from human blood-derived platelets.

115. A method for preparing platelet-rich fibrin, the method comprising the steps of:
i) obtaining platelets from mammalian blood thereby obtaining platelet-rich-plasma (PRP);
ii) adding CaCl₂) to the PRP to a final concentration of greater than 25 mM thereby obtaining a CaCl₂/PRP mixture; and,
iii) agitating the CaCl₂/PRP mixture for less than 6 hours thereby forming a fibrin clot and a supernatant;
iv) adding an anti-fibrinolytic agent to prevent fibrinolysis; and
v) removing the supernatant to obtain the platelet-rich fibrin.

116. The method of claim 115, wherein the mammalian blood is human, equine, porcine, feline, canine, bovine, ovine, murine, rat, simian blood, from a sport animal, from a farm animal, or from a pet.

117. The method of claim 115 or 116, further comprising, prior to ii), concentrating the platelets by removing excess plasma.

118. A platelet-rich fibrin composition produced by the method of any of claims 115-117.

119. The platelet-rich fibrin of claim 118, wherein the fibrin does not include detectable levels of thrombin.

120. A kit comprising (a) a platelet releasate from mammalian blood-derived platelets and (b) instructions for use of the releasate as a cell culture medium supplement.

121. The kit of claim 120, wherein the kit further comprises a cell culture medium.
